SEQUENCE LISTING (52 sequences)

SEQ ID NO: 1 - protein, 93 aa, Homo sapiens
GVTQTPKHLITATGQRVTLRCSPRSGDLSVYWYQQSLDQGLQFLIHYYNGEERAKGNILERFSAQQFPDLHSELNLSSLELGDSALYFCASSV

SEQ ID NO: 2 - protein, 96 aa, Homo sapiens
AVVSQHPSRVICKSGTSVKIECRSLDFQATTMFWYRQFPKQSLMLMATSNEGSKATYEQGVEKDKFLINHASLTLSTLTVTSAHPEDSSFYICSAR

SEQ ID NO: 3 - protein, 93 aa, Homo sapiens
AVSQTPKYLVTQMGNDKSIKCEQNLGHDTMYWYKQDSKKFLKIMFSYNNKELIINETVPNRFSPKSPDKAHLNLHINSLELGDSAVYFCASSQ

SEQ ID NO: 4 - protein, 93 aa, Homo sapiens
DIYQTPRYLVIGTGKKITLECSQTMGHDKMYWYQQDPGMELHLIHYSYGVNSTEKGDLSSESTVSRIRTEHFPLTLESARPSHTSQYLCASSE

SEQ ID NO: 5 - protein, 21 aa, Homo sapiens
MALPVTALLLPLALLLHAARP

SEQ ID NO: 6 - DNA, 63 bp, Homo sapiens
  source 1..63: mol_type = genomic DNA
atggctctgcctgtgacagctctgctgctgcctctggccctgctgctgcatgccgccagacct

SEQ ID NO: 7 - protein, 107 aa, Homo sapiens
DVQMTQSPYNLAASPGESVSINCKASKSINKYLAWYQQKPGKPNKLLIYDGSTLQSGIPSRFSGSGSGTDFTLTIRGLEPEDFGLYYCQQHNEYPPTFGAGTKLELK

SEQ ID NO: 8 - DNA, 321 bp, Homo sapiens
  source 1..321: mol_type = genomic DNA
gacgtgcagatgacccagagcccctacaacctggccgccagccccggcgagagcgtgagcatcaactgcaaggccagcaagagcatcaacaagtacctggcctggtaccagcagaagcccggcaagcccaacaagctgctgatctacgacggcagcaccctgcagagcggcatccccagcaggttcagcggcagcggcagcggcaccgacttcaccctgaccatcaggggcctggagcccgaggacttcggcctgtactactgccagcagcacaacgagtacccccccaccttcggcgccggcaccaagctggagctgaag

SEQ ID NO: 9 - protein, 122 aa, Homo sapiens
QLQLVQSGPELREPGESVKISCKASGYTFTDYIVHWVKQAPGKGLKWMGWINTYTGTPTYADDFEGRFVFSLEASASTANLQISNLKNEDTATYFCARSWRRGIRGIGFDYWGQGVMVTVSS

SEQ ID NO: 10 - DNA, 366 bp, Homo sapiens
  source 1..366: mol_type = genomic DNA
cagctgcagctggtgcagagcggccccgagctgagggagcccggcgagagcgtgaagatcagctgcaaggccagcggctacaccttcaccgactacatcgtgcactgggtgaagcaggcccccggcaagggcctgaagtggatgggctggatcaacacctacaccggcacccccacctacgccgacgacttcgagggcaggttcgtgttcagcctggaggccagcgccagcaccgccaacctgcagatcagcaacctgaagaacgaggacaccgccacctacttctgcgccaggagctggaggaggggcatcaggggcatcggcttcgactactggggccagggcgtgatggtgaccgtgagcagc

SEQ ID NO: 11 - protein, 111 aa, Homo sp.
DIVLTQSPASLAVSLGQRATISCRASKSVSILGTHLIHWYQQKPGQPPKLLIYAASNLESGVPARFSGSGSETVFTLNIHPVEEEDAATYFCQQSIEDPWTFGGGTKLGIK

SEQ ID NO: 12 - DNA, 333 bp, Homo sapiens
  source 1..333: mol_type = genomic DNA
gacatcgtgctgacccagagccccgccagcctggccgtgagcctgggccagagggccaccatcagctgcagggccagcaagagcgtgagcatcctgggcacccacctgatccactggtaccagcagaagcccggccagccccccaagctgctgatctacgccgctagcaacctggagagcggcgtgcccgccaggttcagcggcagcggcagcgagaccgtgttcaccctgaacatccaccccgtggaggaggaggacgccgccacctacttctgccagcagagcatcgaggacccctggaccttcggcggcggcaccaagctgggcatcaag

SEQ ID NO: 13 - protein, 119 aa, Homo sapiens
EVQLQQSVADLVRPGASLKLSCTASGFNIKSAYMHWVIQRPDQGPECLGRIDPATGKTKYAPKFQAKATITADTSSNTAYLQLSSLTSEDTAIYYCTRSLNWDYGLDYWGQGTSVTVSS

SEQ ID NO: 14 - DNA, 357 bp, Homo sapiens
  source 1..357: mol_type = genomic DNA
gaggtgcagctgcagcagagcgtggccgacctggtgaggcccggcgccagcctgaagctgagctgcaccgccagcggcttcaacatcaagagcgcctacatgcactgggtgatccagaggcccgaccagggccccgagtgcctgggcaggatcgaccccgccaccggcaagaccaagtacgcccccaagttccaggccaaggccaccatcaccgccgacaccagcagcaacaccgcctacctgcagctgagcagcctgaccagcgaggacaccgccatctactactgcaccaggagcctgaactgggactacggcctggactactggggccagggcaccagcgtgaccgtgagcagc

SEQ ID NO: 15 - protein, 107 aa, Homo sapiens
ETTVTQSPASLSVATGEKVTIRCISSTDIDDDMNWYQQKSGEPPKLLISEGNTLRPGVPSRFSSSGYGTDFVFTIENMLSEDVADYYCLQSDNMPLTFGAGTKLELK

SEQ ID NO: 16 - DNA, 321 bp, Homo sapiens
  source 1..321: mol_type = genomic DNA
gagaccaccgtgacccagagccccgccagcctgagcgtggccaccggcgagaaggtgaccatcaggtgcatcagcagcaccgacatcgacgacgacatgaactggtaccagcagaagagcggcgagccccccaagctgctgatcagcgagggcaacaccctgaggcccggcgtgcccagcaggttcagcagcagcggctacggcaccgacttcgtgttcaccatcgagaacatgctgagcgaggacgtggccgactactactgcctgcagagcgacaacatgcccctgaccttcggcgccggcaccaagctggagctgaag

SEQ ID NO: 17 - protein, 118 aa, Homo sapiens
EVQLQQSVAELVRPGASVKLSCTASGFNIKNTFMHWVKQRPEQGLEWIGRIDPTNGYTKFAPKFQGKATLTAVTSSNTVYLQLSSLTSEDTAIYYCAHDYDAPWFAYWGQGTLVIVSA

SEQ ID NO: 18 - DNA, 354 bp, Homo sapiens
  source 1..354: mol_type = genomic DNA
gaggtgcagctgcagcagagcgtggccgagctggtgaggcccggcgccagcgtgaagctgagctgcaccgccagcggcttcaacatcaagaacaccttcatgcactgggtgaagcagaggcccgagcagggcctggagtggatcggcaggatcgaccccaccaacggctacaccaagttcgcccccaagttccagggcaaggccaccctgaccgccgtgaccagcagcaacaccgtgtacctgcagctgagcagcctgaccagcgaggacaccgccatctactactgcgcccacgactacgacgccccctggttcgcctactggggccagggcaccctggtgatcgtgagcgcc

SEQ ID NO: 19 - protein, 107 aa, Homo sapiens
DIKMTQSPSSMYASLGERVTITCKASQDINSYLSWFQQKAGKSPKTLIYRANRLVDGVPSRFSGSGSGQDYSLTISSLEYEDMGIYYCLQYDEFPFTFGGGTRLEIK

SEQ ID NO: 20 - DNA, 321 bp, Homo sapiens
  source 1..321: mol_type = genomic DNA
gacattaagatgacccagtccccctcctccatgtatgccagcctcggcgagagagtcaccatcacatgcaaggccagccaagacatcaacagctacctcagctggttccagcagaaagccggcaagagccccaagacactgatctatagggctaatagactggtggacggcgtgcctagcagattttccggcagcggcagcggccaagactattctctgaccatcagctctctggagtacgaggacatgggaatctactactgtctgcagtacgacgagttccccttcaccttcggaggcggcacaagactggaaatcaaa

SEQ ID NO: 21 - protein, 116 aa, Homo sapiens
QVQLQQSGPEVVRPGVSVKISCKGSGYRFTDSAMHWVKQSHAKSLEWIGVISSYNGNTNYNQKFKGKATMTVDKSSSTAYMELARMTSEDSAIYYCARSRDAMDYWGQGTSVTVSS

SEQ ID NO: 22 - DNA, 348 bp, Homo sapiens
  source 1..348: mol_type = genomic DNA
caagtgcagctccagcagtccggacccgaggtggtgaggcccggcgtgagcgtgaagatcagctgcaagggcagcggctataggttcaccgactccgccatgcactgggtgaagcagtcccatgccaagagcctcgagtggatcggcgtgatcagcagctacaacggcaacaccaactacaaccagaagttcaagggcaaggccacaatgaccgtggacaagagcagctccaccgcctacatggagctggccagaatgaccagcgaggatagcgccatctactactgtgctaggtctagagacgccatggactactggggccaaggcacatccgtgaccgtgagctcc

SEQ ID NO: 23 - protein, 5 aa, synthetic construct
GGGGS

SEQ ID NO: 24 - protein, 55 aa, Homo sapiens
FVPVFLPAKPTTTPAPRPPTPAPTIASQPLSLRPEACRPAAGGAVHTRGLDFACD

SEQ ID NO: 25 - protein, 83 aa, Homo sapiens
FVPVFLPAKPTTTPAPRPPTPAPTIASQPLSLRPEACRPAAGGAVHTRGLDFACDIYIWAPLAGTCGVLLLSLVITLYCNHRN

SEQ ID NO: 26 - DNA, 249 bp, Homo sapiens
  source 1..249: mol_type = genomic DNA
ttcgtgcctgtgtttctgcctgccaagcccaccacaacccctgcccctagacctcctacacccgcccctacaatcgccagccagcctctgtctctgaggcccgaggcttgtagacctgctgctggcggagccgtgcacaccagaggactggatttcgcctgcgacatctacatctgggcccctctggccggcacatgtggcgtgctgctgctgagcctcgtgatcaccctgtactgcaaccaccggaac

SEQ ID NO: 27 - protein, 41 aa, Homo sapiens
RSKRSRLLHSDYMNMTPRRPGPTRKHYQPYAPPRDFAAYRS

SEQ ID NO: 28 - DNA, 123 bp, Homo sapiens
  source 1..123: mol_type = genomic DNA
agaagcaagcggagccggctgctgcacagcgactacatgaacatgacccccagacggcctggccccaccagaaagcactaccagccttacgcccctcccagagacttcgccgcctaccggtcc

SEQ ID NO: 29 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - DNA, 336 bp, Homo sapiens
  source 1..336: mol_type = genomic DNA
agagtgaagttcagcagaagcgccgacgcccctgcctatcagcagggccagaaccagctgtacaacgagctgaacctgggcagacgggaagagtacgatgtgctggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 31 - protein, 496 aa, Synthetic construct
MALPVTALLLPLALLLHAARPDVQMTQSPYNLAASPGESVSINCKASKSINKYLAWYQQKPGKPNKLLIYDGSTLQSGIPSRFSGSGSGTDFTLTIRGLEPEDFGLYYCQQHNEYPPTFGAGTKLELKGGGGSGGGGSQLQLVQSGPELREPGESVKISCKASGYTFTDYIVHWVKQAPGKGLKWMGWINTYTGTPTYADDFEGRFVFSLEASASTANLQISNLKNEDTATYFCARSWRRGIRGIGFDYWGQGVMVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - DNA, 1488 bp, Synthetic construct
  source 1..1488: mol_type = other DNA
atggctctgcctgtgacagctctgctgctgcctctggccctgctgctgcatgccgccagacctgacgtgcagatgacccagagcccctacaacctggccgccagccccggcgagagcgtgagcatcaactgcaaggccagcaagagcatcaacaagtacctggcctggtaccagcagaagcccggcaagcccaacaagctgctgatctacgacggcagcaccctgcagagcggcatccccagcaggttcagcggcagcggcagcggcaccgacttcaccctgaccatcaggggcctggagcccgaggacttcggcctgtactactgccagcagcacaacgagtacccccccaccttcggcgccggcaccaagctggagctgaagggaggcggaggctctggcggaggcggctctcagctgcagctggtgcagagcggccccgagctgagggagcccggcgagagcgtgaagatcagctgcaaggccagcggctacaccttcaccgactacatcgtgcactgggtgaagcaggcccccggcaagggcctgaagtggatgggctggatcaacacctacaccggcacccccacctacgccgacgacttcgagggcaggttcgtgttcagcctggaggccagcgccagcaccgccaacctgcagatcagcaacctgaagaacgaggacaccgccacctacttctgcgccaggagctggaggaggggcatcaggggcatcggcttcgactactggggccagggcgtgatggtgaccgtgagcagcttcgtgcctgtgtttctgcctgccaagcccaccacaacccctgcccctagacctcctacacccgcccctacaatcgccagccagcctctgtctctgaggcccgaggcttgtagacctgctgctggcggagccgtgcacaccagaggactggatttcgcctgcgacatctacatctgggcccctctggccggcacatgtggcgtgctgctgctgagcctcgtgatcaccctgtactgcaaccaccggaacagaagcaagcggagccggctgctgcacagcgactacatgaacatgacccccagacggcctggccccaccagaaagcactaccagccttacgcccctcccagagacttcgccgcctaccggtccagagtgaagttcagcagaagcgccgacgcccctgcctatcagcagggccagaaccagctgtacaacgagctgaacctgggcagacgggaagagtacgatgtgctggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 33 - protein, 501 aa, Synthetic construct
MALPVTALLLPLALLLHAARPDVQMTQSPYNLAASPGESVSINCKASKSINKYLAWYQQKPGKPNKLLIYDGSTLQSGIPSRFSGSGSGTDFTLTIRGLEPEDFGLYYCQQHNEYPPTFGAGTKLELKGGGGSGGGGSGGGGSQLQLVQSGPELREPGESVKISCKASGYTFTDYIVHWVKQAPGKGLKWMGWINTYTGTPTYADDFEGRFVFSLEASASTANLQISNLKNEDTATYFCARSWRRGIRGIGFDYWGQGVMVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - DNA, 1503 bp, Synthetic construct
  source 1..1503: mol_type = other DNA
atggctctgcctgtgacagctctgctgctgcctctggccctgctgctgcatgccgccagacctgacgtgcagatgacccagagcccctacaacctggccgccagccccggcgagagcgtgagcatcaactgcaaggccagcaagagcatcaacaagtacctggcctggtaccagcagaagcccggcaagcccaacaagctgctgatctacgacggcagcaccctgcagagcggcatccccagcaggttcagcggcagcggcagcggcaccgacttcaccctgaccatcaggggcctggagcccgaggacttcggcctgtactactgccagcagcacaacgagtacccccccaccttcggcgccggcaccaagctggagctgaagggaggcggaggctctggcggaggcggctctggcggaggcggctctcagctgcagctggtgcagagcggccccgagctgagggagcccggcgagagcgtgaagatcagctgcaaggccagcggctacaccttcaccgactacatcgtgcactgggtgaagcaggcccccggcaagggcctgaagtggatgggctggatcaacacctacaccggcacccccacctacgccgacgacttcgagggcaggttcgtgttcagcctggaggccagcgccagcaccgccaacctgcagatcagcaacctgaagaacgaggacaccgccacctacttctgcgccaggagctggaggaggggcatcaggggcatcggcttcgactactggggccagggcgtgatggtgaccgtgagcagcttcgtgcctgtgtttctgcctgccaagcccaccacaacccctgcccctagacctcctacacccgcccctacaatcgccagccagcctctgtctctgaggcccgaggcttgtagacctgctgctggcggagccgtgcacaccagaggactggatttcgcctgcgacatctacatctgggcccctctggccggcacatgtggcgtgctgctgctgagcctcgtgatcaccctgtactgcaaccaccggaacagaagcaagcggagccggctgctgcacagcgactacatgaacatgacccccagacggcctggccccaccagaaagcactaccagccttacgcccctcccagagacttcgccgcctaccggtccagagtgaagttcagcagaagcgccgacgcccctgcctatcagcagggccagaaccagctgtacaacgagctgaacctgggcagacgggaagagtacgatgtgctggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 35 - protein, 497 aa, Synthetic construct
MALPVTALLLPLALLLHAARPDIVLTQSPASLAVSLGQRATISCRASKSVSILGTHLIHWYQQKPGQPPKLLIYAASNLESGVPARFSGSGSETVFTLNIHPVEEEDAATYFCQQSIEDPWTFGGGTKLGIKGGGGSGGGGSEVQLQQSVADLVRPGASLKLSCTASGFNIKSAYMHWVIQRPDQGPECLGRIDPATGKTKYAPKFQAKATITADTSSNTAYLQLSSLTSEDTAIYYCTRSLNWDYGLDYWGQGTSVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - DNA, 1491 bp, Synthetic construct
  source 1..1491: mol_type = other DNA
atggctctgcctgtgacagctctgctgctgcctctggccctgctgctgcatgccgccagacctgacatcgtgctgacccagagccccgccagcctggccgtgagcctgggccagagggccaccatcagctgcagggccagcaagagcgtgagcatcctgggcacccacctgatccactggtaccagcagaagcccggccagccccccaagctgctgatctacgccgctagcaacctggagagcggcgtgcccgccaggttcagcggcagcggcagcgagaccgtgttcaccctgaacatccaccccgtggaggaggaggacgccgccacctacttctgccagcagagcatcgaggacccctggaccttcggcggcggcaccaagctgggcatcaagggaggcggaggctctggcggaggcggctctgaggtgcagctgcagcagagcgtggccgacctggtgaggcccggcgccagcctgaagctgagctgcaccgccagcggcttcaacatcaagagcgcctacatgcactgggtgatccagaggcccgaccagggccccgagtgcctgggcaggatcgaccccgccaccggcaagaccaagtacgcccccaagttccaggccaaggccaccatcaccgccgacaccagcagcaacaccgcctacctgcagctgagcagcctgaccagcgaggacaccgccatctactactgcaccaggagcctgaactgggactacggcctggactactggggccagggcaccagcgtgaccgtgagcagcttcgtgcctgtgtttctgcctgccaagcccaccacaacccctgcccctagacctcctacacccgcccctacaatcgccagccagcctctgtctctgaggcccgaggcttgtagacctgctgctggcggagccgtgcacaccagaggactggatttcgcctgcgacatctacatctgggcccctctggccggcacatgtggcgtgctgctgctgagcctcgtgatcaccctgtactgcaaccaccggaacagaagcaagcggagccggctgctgcacagcgactacatgaacatgacccccagacggcctggccccaccagaaagcactaccagccttacgcccctcccagagacttcgccgcctaccggtccagagtgaagttcagcagaagcgccgacgcccctgcctatcagcagggccagaaccagctgtacaacgagctgaacctgggcagacgggaagagtacgatgtgctggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 37 - protein, 502 aa, Synthetic construct
MALPVTALLLPLALLLHAARPDIVLTQSPASLAVSLGQRATISCRASKSVSILGTHLIHWYQQKPGQPPKLLIYAASNLESGVPARFSGSGSETVFTLNIHPVEEEDAATYFCQQSIEDPWTFGGGTKLGIKGGGGSGGGGSGGGGSEVQLQQSVADLVRPGASLKLSCTASGFNIKSAYMHWVIQRPDQGPECLGRIDPATGKTKYAPKFQAKATITADTSSNTAYLQLSSLTSEDTAIYYCTRSLNWDYGLDYWGQGTSVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - DNA, 1506 bp, Synthetic construct
  source 1..1506: mol_type = other DNA
atggctctgcctgtgacagctctgctgctgcctctggccctgctgctgcatgccgccagacctgacatcgtgctgacccagagccccgccagcctggccgtgagcctgggccagagggccaccatcagctgcagggccagcaagagcgtgagcatcctgggcacccacctgatccactggtaccagcagaagcccggccagccccccaagctgctgatctacgccgctagcaacctggagagcggcgtgcccgccaggttcagcggcagcggcagcgagaccgtgttcaccctgaacatccaccccgtggaggaggaggacgccgccacctacttctgccagcagagcatcgaggacccctggaccttcggcggcggcaccaagctgggcatcaagggaggcggaggctctggcggaggcggctctggcggaggcggctctgaggtgcagctgcagcagagcgtggccgacctggtgaggcccggcgccagcctgaagctgagctgcaccgccagcggcttcaacatcaagagcgcctacatgcactgggtgatccagaggcccgaccagggccccgagtgcctgggcaggatcgaccccgccaccggcaagaccaagtacgcccccaagttccaggccaaggccaccatcaccgccgacaccagcagcaacaccgcctacctgcagctgagcagcctgaccagcgaggacaccgccatctactactgcaccaggagcctgaactgggactacggcctggactactggggccagggcaccagcgtgaccgtgagcagcttcgtgcctgtgtttctgcctgccaagcccaccacaacccctgcccctagacctcctacacccgcccctacaatcgccagccagcctctgtctctgaggcccgaggcttgtagacctgctgctggcggagccgtgcacaccagaggactggatttcgcctgcgacatctacatctgggcccctctggccggcacatgtggcgtgctgctgctgagcctcgtgatcaccctgtactgcaaccaccggaacagaagcaagcggagccggctgctgcacagcgactacatgaacatgacccccagacggcctggccccaccagaaagcactaccagccttacgcccctcccagagacttcgccgcctaccggtccagagtgaagttcagcagaagcgccgacgcccctgcctatcagcagggccagaaccagctgtacaacgagctgaacctgggcagacgggaagagtacgatgtgctggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 39 - protein, 492 aa, Synthetic construct
MALPVTALLLPLALLLHAARPETTVTQSPASLSVATGEKVTIRCISSTDIDDDMNWYQQKSGEPPKLLISEGNTLRPGVPSRFSSSGYGTDFVFTIENMLSEDVADYYCLQSDNMPLTFGAGTKLELKGGGGSGGGGSEVQLQQSVAELVRPGASVKLSCTASGFNIKNTFMHWVKQRPEQGLEWIGRIDPTNGYTKFAPKFQGKATLTAVTSSNTVYLQLSSLTSEDTAIYYCAHDYDAPWFAYWGQGTLVIVSAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - DNA, 1476 bp, Synthetic construct
  source 1..1476: mol_type = other DNA
atggctctgcctgtgacagctctgctgctgcctctggccctgctgctgcatgccgccagacctgagaccaccgtgacccagagccccgccagcctgagcgtggccaccggcgagaaggtgaccatcaggtgcatcagcagcaccgacatcgacgacgacatgaactggtaccagcagaagagcggcgagccccccaagctgctgatcagcgagggcaacaccctgaggcccggcgtgcccagcaggttcagcagcagcggctacggcaccgacttcgtgttcaccatcgagaacatgctgagcgaggacgtggccgactactactgcctgcagagcgacaacatgcccctgaccttcggcgccggcaccaagctggagctgaagggaggcggaggctctggcggaggcggctctgaggtgcagctgcagcagagcgtggccgagctggtgaggcccggcgccagcgtgaagctgagctgcaccgccagcggcttcaacatcaagaacaccttcatgcactgggtgaagcagaggcccgagcagggcctggagtggatcggcaggatcgaccccaccaacggctacaccaagttcgcccccaagttccagggcaaggccaccctgaccgccgtgaccagcagcaacaccgtgtacctgcagctgagcagcctgaccagcgaggacaccgccatctactactgcgcccacgactacgacgccccctggttcgcctactggggccagggcaccctggtgatcgtgagcgccttcgtgcctgtgtttctgcctgccaagcccaccacaacccctgcccctagacctcctacacccgcccctacaatcgccagccagcctctgtctctgaggcccgaggcttgtagacctgctgctggcggagccgtgcacaccagaggactggatttcgcctgcgacatctacatctgggcccctctggccggcacatgtggcgtgctgctgctgagcctcgtgatcaccctgtactgcaaccaccggaacagaagcaagcggagccggctgctgcacagcgactacatgaacatgacccccagacggcctggccccaccagaaagcactaccagccttacgcccctcccagagacttcgccgcctaccggtccagagtgaagttcagcagaagcgccgacgcccctgcctatcagcagggccagaaccagctgtacaacgagctgaacctgggcagacgggaagagtacgatgtgctggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 41 - protein, 497 aa, Synthetic construct
MALPVTALLLPLALLLHAARPETTVTQSPASLSVATGEKVTIRCISSTDIDDDMNWYQQKSGEPPKLLISEGNTLRPGVPSRFSSSGYGTDFVFTIENMLSEDVADYYCLQSDNMPLTFGAGTKLELKGGGGSGGGGSGGGGSEVQLQQSVAELVRPGASVKLSCTASGFNIKNTFMHWVKQRPEQGLEWIGRIDPTNGYTKFAPKFQGKATLTAVTSSNTVYLQLSSLTSEDTAIYYCAHDYDAPWFAYWGQGTLVIVSAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - DNA, 1491 bp, Synthetic construct
  source 1..1491: mol_type = other DNA
atggctctgcctgtgacagctctgctgctgcctctggccctgctgctgcatgccgccagacctgagaccaccgtgacccagagccccgccagcctgagcgtggccaccggcgagaaggtgaccatcaggtgcatcagcagcaccgacatcgacgacgacatgaactggtaccagcagaagagcggcgagccccccaagctgctgatcagcgagggcaacaccctgaggcccggcgtgcccagcaggttcagcagcagcggctacggcaccgacttcgtgttcaccatcgagaacatgctgagcgaggacgtggccgactactactgcctgcagagcgacaacatgcccctgaccttcggcgccggcaccaagctggagctgaagggaggcggaggctctggcggaggcggctctggcggaggcggctctgaggtgcagctgcagcagagcgtggccgagctggtgaggcccggcgccagcgtgaagctgagctgcaccgccagcggcttcaacatcaagaacaccttcatgcactgggtgaagcagaggcccgagcagggcctggagtggatcggcaggatcgaccccaccaacggctacaccaagttcgcccccaagttccagggcaaggccaccctgaccgccgtgaccagcagcaacaccgtgtacctgcagctgagcagcctgaccagcgaggacaccgccatctactactgcgcccacgactacgacgccccctggttcgcctactggggccagggcaccctggtgatcgtgagcgccttcgtgcctgtgtttctgcctgccaagcccaccacaacccctgcccctagacctcctacacccgcccctacaatcgccagccagcctctgtctctgaggcccgaggcttgtagacctgctgctggcggagccgtgcacaccagaggactggatttcgcctgcgacatctacatctgggcccctctggccggcacatgtggcgtgctgctgctgagcctcgtgatcaccctgtactgcaaccaccggaacagaagcaagcggagccggctgctgcacagcgactacatgaacatgacccccagacggcctggccccaccagaaagcactaccagccttacgcccctcccagagacttcgccgcctaccggtccagagtgaagttcagcagaagcgccgacgcccctgcctatcagcagggccagaaccagctgtacaacgagctgaacctgggcagacgggaagagtacgatgtgctggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 43 - protein, 490 aa, Synthetic construct
MALPVTALLLPLALLLHAARPDIKMTQSPSSMYASLGERVTITCKASQDINSYLSWFQQKAGKSPKTLIYRANRLVDGVPSRFSGSGSGQDYSLTISSLEYEDMGIYYCLQYDEFPFTFGGGTRLEIKGGGGSGGGGSQVQLQQSGPEVVRPGVSVKISCKGSGYRFTDSAMHWVKQSHAKSLEWIGVISSYNGNTNYNQKFKGKATMTVDKSSSTAYMELARMTSEDSAIYYCARSRDAMDYWGQGTSVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 495 aa, Synthetic construct
MALPVTALLLPLALLLHAARPDIKMTQSPSSMYASLGERVTITCKASQDINSYLSWFQQKAGKSPKTLIYRANRLVDGVPSRFSGSGSGQDYSLTISSLEYEDMGIYYCLQYDEFPFTFGGGTRLEIKGGGGSGGGGSGGGGSQVQLQQSGPEVVRPGVSVKISCKGSGYRFTDSAMHWVKQSHAKSLEWIGVISSYNGNTNYNQKFKGKATMTVDKSSSTAYMELARMTSEDSAIYYCARSRDAMDYWGQGTSVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - DNA, 1485 bp, Synthetic construct
  source 1..1485: mol_type = other DNA
atggctctgcccgtcacagctctgctgctgcctctggctctgctgctgcacgctgctagacccgacattaagatgacccagtccccctcctccatgtatgccagcctcggcgagagagtcaccatcacatgcaaggccagccaagacatcaacagctacctcagctggttccagcagaaagccggcaagagccccaagacactgatctatagggctaatagactggtggacggcgtgcctagcagattttccggcagcggcagcggccaagactattctctgaccatcagctctctggagtacgaggacatgggaatctactactgtctgcagtacgacgagttccccttcaccttcggaggcggcacaagactggaaatcaaaggaggaggaggatccggaggcggaggaagcggaggcggaggaagccaagtgcagctccagcagtccggacccgaggtggtgaggcccggcgtgagcgtgaagatcagctgcaagggcagcggctataggttcaccgactccgccatgcactgggtgaagcagtcccatgccaagagcctcgagtggatcggcgtgatcagcagctacaacggcaacaccaactacaaccagaagttcaagggcaaggccacaatgaccgtggacaagagcagctccaccgcctacatggagctggccagaatgaccagcgaggatagcgccatctactactgtgctaggtctagagacgccatggactactggggccaaggcacatccgtgaccgtgagctcctttgtgcccgtgtttctgcccgccaagcctacaaccacacccgctcctagacctcccacccccgcccctaccatcgcttcccagcctctgtctctgagacccgaggcttgcagacccgccgctggaggcgctgtgcacaccagaggactggatttcgcctgcgacatctacatctgggcccctctggccggcacatgtggcgtgctgctgctgagcctcgtgatcaccctgtactgcaaccaccggaacagaagcaagcggagccggctgctgcacagcgactacatgaacatgacccccagacggcctggccccaccagaaagcactaccagccttacgcccctcccagagacttcgccgcctaccggtccagagtgaagttcagcagaagcgccgacgcccctgcctatcagcagggccagaaccagctgtacaacgagctgaacctgggcagacgggaagagtacgatgtgctggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 46 - protein, 47 aa, Homo sapiens
RFSVVKRGRKKLLYIFKQPFMRPVQTTQEEDGCSCRFPEEEEGGCEL

SEQ ID NO: 47 - DNA, 141 bp, Homo sapiens
  source 1..141: mol_type = genomic DNA
cgtttctctgttgttaaacggggcagaaagaagctcctgtatatattcaaacaaccatttatgagaccagtacaaactactcaagaggaagatggctgtagctgccgatttccagaagaagaagaaggaggatgtgaactg

SEQ ID NO: 48 - DNA, 501 bp, Homo sapiens
  source 1..501: mol_type = genomic DNA
gggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcattctccgggcctttcg

SEQ ID NO: 49 - protein, 21 aa, Homo sapiens
IYIWAPLAGTCGVLLLSLVIT

SEQ ID NO: 50 - protein, 7 aa, Homo sapiens
LYCNHRN

SEQ ID NO: 51 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 52 - DNA, 1470 bp, Synthetic construct
  source 1..1470: mol_type = other DNA
atggctctgcccgtcacagctctgctgctgcctctggctctgctgctgcacgctgctagacccgacattaagatgacccagtccccctcctccatgtatgccagcctcggcgagagagtcaccatcacatgcaaggccagccaagacatcaacagctacctcagctggttccagcagaaagccggcaagagccccaagacactgatctatagggctaatagactggtggacggcgtgcctagcagattttccggcagcggcagcggccaagactattctctgaccatcagctctctggagtacgaggacatgggaatctactactgtctgcagtacgacgagttccccttcaccttcggaggcggcacaagactggaaatcaaaggaggaggaggatccggaggcggaggaagccaagtgcagctccagcagtccggacccgaggtggtgaggcccggcgtgagcgtgaagatcagctgcaagggcagcggctataggttcaccgactccgccatgcactgggtgaagcagtcccatgccaagagcctcgagtggatcggcgtgatcagcagctacaacggcaacaccaactacaaccagaagttcaagggcaaggccacaatgaccgtggacaagagcagctccaccgcctacatggagctggccagaatgaccagcgaggatagcgccatctactactgtgctaggtctagagacgccatggactactggggccaaggcacatccgtgaccgtgagctcctttgtgcccgtgtttctgcccgccaagcctacaaccacacccgctcctagacctcccacccccgcccctaccatcgcttcccagcctctgtctctgagacccgaggcttgcagacccgccgctggaggcgctgtgcacaccagaggactggatttcgcctgcgacatctacatctgggcccctctggccggcacatgtggcgtgctgctgctgagcctcgtgatcaccctgtactgcaaccaccggaacagaagcaagcggagccggctgctgcacagcgactacatgaacatgacccccagacggcctggccccaccagaaagcactaccagccttacgcccctcccagagacttcgccgcctaccggtccagagtgaagttcagcagaagcgccgacgcccctgcctatcagcagggccagaaccagctgtacaacgagctgaacctgggcagacgggaagagtacgatgtgctggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

(19) United States
(12) Patent Application Publication
(10) Pub. No.: US 2026/0355238 A1
(43) Pub. Date: Nov. 12, 2026

(54) CHIMERIC ANTIGEN RECEPTOR (CAR) TO A TCR BETA-CHAIN VARIABLE REGION

(71) Applicant: Imperial College Innovations Limited, London (GB)

(72) Inventors: Anastasios Karadimitris, London (GB); Aileen Rowan, London (GB)

(21) Appl. No.: 19/104,798

(22) PCT Filed: Aug. 22, 2023
(86) PCT No.: PCT/GB2023/052187

§ 371 Date: Feb. 19, 2025

(30) Foreign Application Priority Data
Aug. 23, 2022 (GR) 20220100703

(51) Int. Cl. A61K 35/17; A61K 40/11; A61K 40/15; A61K 40/31; A61K 40/42; A61K 40/50; A61P 35/02

(52) U.S. Cl. CPC A61K 35/17; A61K 40/11; A61K 40/15; A61K 40/31; A61K 40/421; A61K 40/50; A61P 35/02; A61K 2239/13; A61K 2239/17; A61K 2239/21; A61K 2239/48

(57) ABSTRACT

The invention relates to chimeric antigen receptors (CARs) per se, to CAR constructs, and to T-cell receptor (TCR) beta-chain variable region beta (TCRVβ) CARs. The invention relates to the use of the CARs in immunotherapy, for example in treating, preventing or ameliorating cancer, such as T-cell malignancies, and also conditions caused or implicated by pathogenic T cells, such as autoimmune diseases. The invention is especially concerned with conventional T cells and invariant natural killer T (iNKT) cells expressing anti-TCRVβ CARs and methods for making the same. The invention extends to nucleic acids and vectors encoding the anti-TCRVβ CARs, and to pharmaceutical compositions comprising the constructs and cells, and to medical uses of the compositions, anti-TCRVβ CAR constructs and T cells and iNKT cells expressing anti-TCRVβ CARs.

The present invention relates to chimeric antigen receptors (CARs) per se, and to CAR constructs, and particularly, although not exclusively, to T-cell receptor (TCR) beta-chain variable region beta (TCRVβ) CARs. The invention relates to the use of the CARs in immunotherapy, for example in treating, preventing or ameliorating cancer, such as T-cell malignancies, and also conditions caused or implicated by pathogenic T cells, such as autoimmune diseases. The invention is especially concerned with conventional T cells and invariant natural killer T (iNKT) cells expressing anti-TCRVβ CARs and methods for making the same. The invention extends to nucleic acids and vectors encoding the anti-TCRVβ CARs, and to pharmaceutical compositions comprising the constructs and cells, and to medical uses of the compositions, anti-TCRVβ CAR constructs and T cells and iNKT cells expressing anti-TCRVβ CARs.

Human T cell leukaemia virus type-1 (HTLV-1)-associated-adult T cell leukaemia/lymphoma (ATL), and the majority of other T cell lymphomas (TCL), have a poor prognosis with current treatments. Carriers of HTLV-1, a virus that is endemic in many parts of the world, have a 5% risk of developing ATL in their lifetime^{2,3}.

Expression of cell surface T-cell receptor (TCR) α/β chain heterodimers is restricted to normal, to non-malignant pathogenic and to malignant T-cells. TCR variable region β (TCRVβ) subunits are encoded by TRBV genes, of which there are 23 families and 114 alleles in humans with each family comprising 0.5-9% of the normal T-cell repertoire, with TCRVβ2 being the most frequent. Signals from the TCR determine cell fate in normal T cells, and components of the TCR signalling pathway are frequently mutated in TCL including ATL, indicating a driving role for the TCR in TCL oncogenesis. Expression of surface TCR is stable in blood and lymph node lymphoma T cells⁴⁻⁶ as well as in non-malignant pathogenic T cells.

CAR-T therapeutic approaches in B cell leukaemias and lymphomas targeting pan-B cell markers, such CD19 and CD20, can induce sustained clinical remissions in up to 40% of patients with relapsed/refractory disease, at the expense of clinically tolerable pan-B-cell depletion and hypoglobulinaemia⁷. However, pan-T cell depletion would result in profound immunosuppression that is not clinically tolerable. Since most TCL are CD4+, anti-CD4 CAR-T treatment has also been proposed for these lymphomas (see, for example, the clinical trial NCT03829540). Unless rescued by a stem cell transplant, such an approach would also result in immunosuppression akin to acquired immunodeficiency syndrome. Other CAR targets include CD5 and CD7; however, these are widely expressed on healthy T cells and can cause a fratricide reaction, unless CAR effector cells are additionally genetically edited to lack expression of these markers⁸. Recently, efficacy using CAR-T cells to target the TCRβ chain constant region 1 which is expressed in ~40-60% of TCRαβ lymphocytes was reported⁹. It remains to be seen what the impact of depleting 40-60% of the T cell repertoire will have on an individuals' immune status. It may be that, as reported in murine models, loss of half of the TCR sequences significantly impairs immunity in humans¹⁰. Current anti-T cell lymphoma CAR-based immunotherapies eliminate either the whole or 50% of healthy T cells, thus rendering the treated patient with T cell lymphoma severely or profoundly immunosuppressed.

There is, therefore, a need to provide improved immunotherapies for T-cell malignancies, such as T-cell lymphoma, including ATL, and also for treating diseases, such as certain autoimmune diseases caused directly or indirectly by pathogenic, non-malignant T cells, as well as for targeting normal, non-pathogenic T cells.

In order to fight malignant and non-malignant pathogenic T cells, as well as non-pathogenic T cells, the inventors have developed CAR-iNKT (invariant natural killer T) cells as an ‘off-the-shelf’ allogenic immunotherapy. iNKT cells are a rare (i.e. they represent less than 0.1% of the T-cells population) and evolutionarily conserved subset of T cells which share features of innate and adaptive immune responses¹¹⁻¹³. In humans, iNKT are characterised by the expression of an invariant TCRVα24Jα18 chain which mostly pairs with diverse TCRVβ11 chains (iTCR)¹⁴. INKT cells are restricted by CD1d, a non-polymorphic, glycolipid-presenting HLA class I-like molecule expressed on monocytes, macrophages dendritic cells, B cells, thymocytes and some epithelial tissues¹⁵. In addition, iNKT cells have a memory effector phenotype and can migrate to extra-lymphoid tissues¹⁸⁻²⁰ where they modulate a variety of immune responses, including anti-tumour and anti-pathogen responses^{21,22}.

Since iNKT cells protect against acute graft-versus-host disease (aGVHD)²⁵⁻²⁸, CAR-iNKT-cell immunotherapy can be sourced from allogeneic healthy donors as ‘off-the shelf’ treatment without need for deletion of the endogenous TCR as is the case with conventional T cells. Conversely, the standard autologous CAR-T immunotherapy can be limited by financial and logistical challenges and sub-optimal fitness of patient-derived T cells.

Based on the above considerations, the inventors have explored targeting of ATL/TCL-specific TCRVβ chains through CAR engineering of T and iNKT cells. To address the pressing clinical need for new therapeutic approaches, the inventors have developed exemplary chimeric antigen receptors (CARs) which target a group of subunits of the T cell receptor (TCR) β chain, which is constitutively expressed by mature T cell leukaemias and lymphomas.

Thus, in a first aspect of the invention, there is provided a chimeric antigen receptor (CAR) construct specific for a T-cell receptor (TCR) beta-chain variable region (Vβ) subunit of a T cell.

As shown in the Examples, the inventors have developed various embodiments of exemplary CARs specific for several TCR Vβ subunits according to the invention (referred herein as clone BL37.2 for TCR Vβ1, MPB2D5 for TCR Vβ2, FIN9 for TCR Vβ9 and C21 for TCR Vβ11). Advantageously, using conventional T cells and innate natural killer (INKT) cells as CAR effector cells, the inventors have surprisingly demonstrated that anti-Vβ CAR-T cells and CAR-iNKT cells harbouring the CAR of the invention can successfully kill in-vitro expanded primary T cells and ex-vivo adult T cell leukaemia (ATL) cells, which express the corresponding TCRVβ subunit, with surprisingly minimal off target killing of healthy T cells which express other TCRVβ subunits. The inventors have successfully demonstrated that CAR-iNKT activity was significantly enhanced when challenged with CD1d-expressing targets that had been pulsed with α-Galactosylceramide, a selective ligand of iNKT cells that leads to their activation. Ligation of the anti-Vβ CAR induced degranulation, secretion of IFN-γ and TNF-α, and upregulation of expression of Perforin and Granzyme by T and iNKT effectors. Targeting T cells, which express a single Vβ subunit, spared virus-specific CTL. Furthermore, in a short in vitro assay, the inventors have shown that CAR-mediated killing had no effect on the expression of the virus which causes ATL, Human T cell leukaemia virus type-1. Finally, anti-TCRVβ CAR-INKT (but not anti-CD19 CAR-INKT) significantly (p<0.01) reduced tumour size in a sub-cutaneous model of T cell lymphoma. Tumour-bearing mice that received iNKT effectors showed no signs of graft-versus-host disease.

Accordingly, through these experiments, the inventors have surprisingly demonstrated that CARs targeting TCRVβ subunits can efficiently kill malignant T cell clones with minimal off-target cytotoxicity. CAR-iNKT effectors displayed enhanced killing activity in the presence of iNKT ligands, reducing the risk of acute graft versus host disease and are suitable for use as an off-the shelf product for infusion to third party donors.

In one embodiment, the CAR construct is specific for a T-cell receptor (TCR) beta-chain variable region (Vβ) subunit of a normal, non-pathogenic T cell.

In another embodiment, however, the CAR construct is specific for a T-cell receptor (TCR) beta-chain variable region (Vβ) subunit of a pathogenic T cell.

Pathogenic T cells are clonal in nature with tumour cells sharing expression of a single TCRVβ subunit, with no bias or preference in the usage of TCRVβ subunit families⁴⁻⁶. Advantageously, targeting the specific TCRVβ subunit expressed by pathogenic T cells in a clonal manner provides a highly selective and tumour-specific therapeutic solution with “on-target off-tumour” toxicity limited to less than 5-9% of normal T-cells. As the expression of each TCRVβ subunit is restricted to less than 9% of healthy T cells, this approach avoids treatment-induced immunosuppression and immune dysregulation, which might be caused by alternative CAR-based immunotherapies for ATL/TCL, because the novel construct preserves more than 90% of healthy T cells.

Based on the above considerations, therefore, the inventors have explored targeting of ATL/TCL-specific TCRVβ chains through CAR engineering of T and iNKT cells. To address the pressing clinical need for new therapeutic approaches, the inventors have developed chimeric antigen receptors (CAR) which target the T cell receptor (TCR) β chain, which is constitutively expressed by mature T cell leukaemias and lymphomas.

Preferably, therefore, the CAR construct of the invention is specific for the TCRVβ subunit of a pathogenic T cell.

A “pathogenic T cell” can refer to a T cell expressed in a clinical disorder, such as a T cell malignancy, including TCL and ATL, infections, as well as auto-immune diseases. Hence, the anti-TCRVβ CAR can target in a highly selective manner, pathogenic T cells, such as those in involved in T cell lymphomas and T cell leukaemias, as well as those T cells causing auto-immune disease.

Thus, the pathogenic T cell may be a malignant, pathogenic T cell. Alternatively, the pathogenic T cell may be a non-malignant, pathogenic T cell.

Healthy T cells express 23 different families of TCRVβ chain molecules.

Advantageously, each T cell expresses only one of the TCRVβ chains. Thus, pathological T cells, such as those in cancers, namely T cell lymphomas and T cell leukaemias, as well as those causing auto-immune disease, only express one type of TCRVβ chain. However, the same TCRVβ chain that is expressed in pathological T cells will also be expressed in less than 10% of normal, healthy T cells. Therefore, anti-TCRVβ CARs that selectively target pathological T cells will also target less than 10% of the patient's healthy T cells, although more than 90% of the patient's healthy T cells and T cell-dependent immunity will be left intact. Accordingly, in some embodiments, healthy T cells may be targeted by the CAR construct of the invention.

Table 1 below lists TCRVβ subunits on T-cells, with the associated encoding gene, and any one or more of these may be targeted by the CAR construct of the invention.

TABLE 1
Beta-chain variable sub-units (Vβ) on T cells

| | TRBV | TRBV |
|---|---|---|
| Protein name | gene name | allele name |
| TCRVβ1 | TRBV9 | TRBV9*01 |
| | | TRBV9*02 |
| | | TRBV9*03 |
| TCRVβ2 | TRBV20-1 | TRBV20-1*01 |
| | | TRBV20-1*02 |
| | | TRBV20-1*03 |
| | | TRBV20-1*04 |
| | | TRBV20-1*05 |
| | | TRBV20-1*06 |
| | | TRBV20-1*07 |
| TCRVβ3/3.1 | TRBV28 | TRBV28*01 |
| TCRVβ4 | TRBV29-1 | TRBV29-1*01 |
| | | TRBV29-1*02 |
| | | TRBV29-1*03 |
| TCRVβ5.1 | TRBV5-1 | TRBV5-1*01 |
| TCRVβ5.1 | | TRBV5-1*02 |
| TCRVβ5.2/5.3 | TRBV5-5 | TRBV5-5*01 |
| | | TRBV5-5*02 |
| | | TRBV5-5*03 |
| TCRVβ5.2 | TRBV5-6 | TRBV5-6*01 |
| TCRVβ6.7 | TRBV7-2 | TRBV7-2*01 |
| | | TRBV7-2*02 |
| | | TRBV7-2*03 |
| | | TRBV7-2*04 |
| TCRVβ7.1 | TRBV4-1 | TRBV4-1*01 |
| | | TRBV4-1*02 |
| TCRVβ7.1 | TRBV4-2 | TRBV4-2*01 |
| | | TRBV4-2*02 |
| TCRVβ7.1 | TRBV4-3 | TRBV4-3*01 |
| | | TRBV4-3*02 |
| | | TRBV4-3*03 |
| TCRVβ7.2 | | TRBV4-3*04 |
| TCRVβ8.1/8.2 | TRBV12-3 | TRBV12-3*01 |
| | TRBV12-4 | TRBV12-4*01 |
| | | TRBV12-4*02 |
| TCRVβ8 | TRBV12-5 | TRBV12-5*01 |
| TCRVβ9 | TRBV3-1 | TRBV3-1*01 |
| | | TRBV3-1*02 |
| TCRVβ11 | TRBV25-1 | TRBV25-1*01 |
| TCRVβ12 | TRBV10-1 | TRBV10-1*01 |
| | | TRBV10-1*02 |
| TCRVβ12 | TRBV10-2 | TRBV10-2*01 |
| | | TRBV10-2*02 |
| TCRVβ12 | TRBV10-3 | TRBV10-3*01 |
| TCRVβ12 | | TRBV10-3*02 |
| | | TRBV10-3*03 |
| | | TRBV10-3*04 |
| TCRVβ13.1, 13.3 | TRBV6-1 | TRBV6-1*01 |
| TCRVβ13.2 | TRBV6-2 | TRBV6-2*01 |
| TCRVβ13.1, 13.3 | TRBV6-5 | TRBV6-5*01 |
| TCRVβ13.1 | TRBV6-6 | TRBV6-6*04 |
| | | TRBV6-6*05 |
| TCRVβ13.1 | TRBV6-9 | TRBV6-9*01 |
| TCRVβ13.6 | TRBV6-6 | TRBV6-6*01 |
| | | TRBV6-6*02 |
| | | TRBV6-6*03 |
| TCRVβ14 | TRBV27 | TRBV27*01 |
| TCRVβ16 | TRBV14 | TRBV14*01 |
| | | TRBV14*02 |
| TCRVβ17 | TRBV19 | TRBV19*01 |
| | | TRBV19*02 |
| | | TRBV19*03 |
| TCRVβ18 | TRBV18 | TRBV18*01 |
| TCRVβ20 | TRBV30 | TRBV30*01 |
| | | TRBV30*02 |
| | | TRBV30*04 |
| | | TRBV30*05 |
| TCRVβ21.3 | TRBV11-2 | TRBV11-2*01 |
| | | TRBV11-2*02 |
| | | TRBV11-2*03 |
| TCRVβ22 | TRBV2 | TRBV2*01 |
| | | TRBV2*02 |
| | | TRBV2*03 |
| TCRVβ23 | TRBV13 | TRBV13*01 |
| | | TRBV13*02 |
| n/a | TRBV5-4 | TRBV5-4*01 |
| | | TRBV5-4*02 |
| | | TRBV5-4*03 |
| | | TRBV5-4*04 |
| n/a | TRBV5-8 | TRBV5-8*01 |
| | | TRBV5-8*02 |
| n/a | TRBV6-3 | TRBV6-3*01 |
| n/a | TRBV6-4 | TRBV6-4*01 |
| | | TRBV6-4*02 |
| n/a | TRBV6-8 | TRBV6-8*01 |
| n/a | TRBV7-3 | TRBV7-3*01 |
| | | TRBV7-3*04 |
| | | TRBV7-3*05 |
| n/a | TRBV7-4 | TRBV7-4*01 |
| n/a | TRBV7-6 | TRBV7-6*01 |
| | | TRBV7-6*02 |
| n/a | TRBV7-7 | TRBV7-7*01 |
| | | TRBV7-7*02 |
| n/a | TRBV7-8 | TRBV7-8*01 |
| | | TRBV7-8*02 |
| | | TRBV7-8*03 |
| n/a | TRBV7-9 | TRBV7-9*01 |
| | | TRBV7-9*02 |
| | | TRBV7-9*03 |
| | | TRVB7-9*04 |
| | | TRBV7-9*05 |
| | | TRBV7-9*06 |
| | | TRBV7-9*07 |
| n/a | TRBV11-1 | TRBV11-1*01 |
| n/a | TRBV11-3 | TRBV11-3*01 |
| | | TRBV11-3*02 |
| | | TRBV11-3*03 |
| | | TRBV11-3*04 |
| n/a | TRBV15 | TRBV15*01 |
| | | TRBV15*02 |
| | | TRBV15*03 |
| n/a | TRBV16 | TRBV16*01 |
| | | TRBV16*03 |
| n/a | TRBV24-1 | TRBV24-1*01 |

Preferably, therefore, the Vβ subunit may be selected from a group of Vβ subunits shown in Table 1.

The frequency of TCRVβ chain family use in T-cell lymphoma is similar to that in the normal T cell repertoire as shown in Table 1. In a preferred embodiment, the CAR construct targets a TCR Vβ subunit on a T-cell wherein the subunit is any one of those in the left hand column of Table 1.

As described in the examples, the inventors have made four embodiments of CAR construct, each of which is specific for TCR-Vβ1, TCR-Vβ2, TCR-Vβ9 and TCR-Vβ11, respectively. Preferably, therefore, the CAR construct targets a TCR Vβ subunit on a T-cell selected from a group consisting of the following Vβ subunits: TCR-Vβ1, TCR-Vβ2, TCR-Vβ9 and TCR-Vβ11.

Thus, preferably the CAR construct is specific for TCR-Vβ1. One embodiment of the polypeptide sequence of TCR Vβ1 subunit (encoded by H. sapiens TCRBV9 of which there are three alleles, TRBV9*01 to *039-UniProtKB: A0A0B4J1U6) is represented herein as SEQ ID No: 1, as follows:

[SEQ ID No: 1]
GVTQTPKHLITATGQRVTLRCSPRSGDLSVYWYQQSLDQGLQFLIHYYNG
EERAKGNILERFSAQQFPDLHSELNLSSLELGDSALYFCASSV

Therefore, preferably the CAR construct is specific for the TCR-Vβ1 subunit, which comprises an amino acid sequence substantially as set out in SEQ ID No:1, or a variant or fragment thereof.

Preferably, the CAR construct is specific for TCR-Vβ2. One embodiment of the polypeptide sequence of TCR Vβ2 subunit (encoded by H. sapiens TCRBV20-1 of which there are seven alleles, TRBV20-1*01 to *07-UniProtKB: A0A075B6N2) is represented herein as SEQ ID No: 2, as follows:

[SEQ ID No: 2]
AVVSQHPSRVICKSGTSVKIECRSLDFQATTMFWYRQFPKQSLMLMATSN
EGSKATYEQGVEKDKFLINHASLTLSTLTVTSAHPEDSSFYICSAR

Therefore, preferably the CAR construct is specific for the TCR-Vβ2 subunit, which comprises an amino acid sequence substantially as set out in SEQ ID No:2, or a variant or fragment thereof.

Preferably, the CAR construct is specific for TCR-Vβ9. One embodiment of the polypeptide sequence of TCR Vβ9 subunit (encoded by H. sapiens TCRBV3-1 of which there are two alleles, TRBV3-1*01 and *02-UniProtKB/Swiss-Prot: A0A576) is represented herein as SEQ ID No: 3, as follows:

[SEQ ID No: 3]
AVSQTPKYLVTQMGNDKSIKCEQNLGHDTMYWYKQDSKKFLKIMFSYNNK
ELIINETVPNRFSPKSPDKAHLNLHINSLELGDSAVYFCASSQ

Therefore, preferably the CAR construct is specific for the TCR-Vβ9 subunit, which comprises an amino acid sequence substantially as set out in SEQ ID No:3, or a variant or fragment thereof.

Preferably, the CAR construct is specific for TCR-Vβ11. One embodiment of the polypeptide sequence of TCR Vβ11 subunit (encoded by H. sapiens TCRBV25-1*01 of which there is one allele—UniProtKB: A0A075B6N4) is represented herein as SEQ ID No: 4, as follows:

[SEQ ID No: 4]
DIYQTPRYLVIGTGKKITLECSQTMGHDKMYWYQQDPGMELHLIHYSYGV
NSTEKGDLSSESTVSRIRTEHFPLTLESARPSHTSQYLCASSE

Therefore, preferably the CAR construct is specific for the TCR-Vβ11 subunit, which comprises an amino acid sequence substantially as set out in SEQ ID No:4, or a variant or fragment thereof.

Preferably, in one embodiment, the CAR construct comprises:
a) a signalling peptide;
b) an antigen-binding domain or moiety specific for the TCR-Vβ subunit, preferably an anti-TCRVβ antibody or a functional fragment thereof specific for the TCR-Vβ subunit, and more preferably a single-chain variable fragment (scFv) domain of an anti-TCRVβ antibody;
c) a hinge region or hinge domain, preferably comprising a transmembrane domain, and most preferably a transmembrane domain and cytoplasmic region;
d) a primary stimulatory (or signalling) domain; and/or
e) a co-stimulatory domain, preferably two or more co-stimulatory domains.

Surprisingly, the inventors have demonstrated that the combination of each of these components generates an anti-TCRVβ CAR construct with highly selective activity against the cognate TCRVβ family chain and significant activity against T cell lymphoma and leukaemia activity in vitro and in vivo.

Thus, preferably the CAR construct comprises a signalling peptide. Advantageously, the signalling peptide is configured to lead the CAR (i.e. which is a fusion protein) to the outer membrane of an effector cell (i.e. T-cell) expressing the CAR construct. The signalling peptide of the CAR construct may be a native immunoglobulin gene signalling peptide. Preferably, however, the signalling peptide comprises human CD8α, or a fragment or variant thereof. CD8α is highly expressed in human T cells. Advantageously, therefore, when the CAR construct is expressed in a human effector T cell, the human CD8a provides optimal expression of the CAR construct in the human effector cell.

In one embodiment, the human CD8a signalling peptide can have an amino acid sequence referred to herein as SEQ ID No:5, as follows:

[SEQ ID No: 5]
MALPVTALLLPLALLLHAARP

Preferably, therefore, the CAR construct comprises a signalling peptide having an amino acid sequence substantially as set out in SEQ ID No:5, or a fragment or variant thereof.

In one embodiment, a nucleotide sequence encoding the signalling peptide is referred to herein as SEQ ID No:6, as follows:

[SEQ ID No: 6]
atggctctgcctgtgacagctctgctgctgcctctggccctgctgctgca
tgccgccagacct

Preferably, therefore, the signalling peptide is encoded by a nucleotide sequence substantially as set out in SEQ ID No: 6, or a fragment or variant thereof.

Preferably, the antigen-binding domain or moiety specific for the TCR-Vβ subunit comprises an anti-TCRVβ antibody or a functional fragment thereof that is specific for the TCR-Vβ subunit. More preferably, the antigen-binding domain specific for the TCR-Vβ subunit comprises a single-chain variable fragment (scFv) domain of an anti-TCRVB antibody. Preferably, the antigen-binding domain is disposed C-term of the signalling peptide.

The skilled person is aware that the scFv is a fusion protein that comprises the variable regions of the heavy (V_{H}) and light chains (V_{L}) of a given antibody. In the context of the present invention, the antibody is one that is specific for a TCRVβ subunit.

As discussed herein, there are 23 different families of TCRVβ chain molecules. It will be appreciated that any human TCRVβ chain may be targeted by the CAR construct of the invention. TCRβ chain sequences are readily available in public databases. For example, any of the TCRVβ listed in Tables 1 and 2, which provides a summary of TCR gene (TRBV) and protein (TCRVβ), and indicates the proteins for which monoclonal antibodies are available or can be developed, may be targeted by the CAR construct.

Therefore, in one embodiment, scFv domain of an anti-TCRVβ antibody is selected from any one of the TCRVβ chain genes and proteins recited in Table 1 and 2.

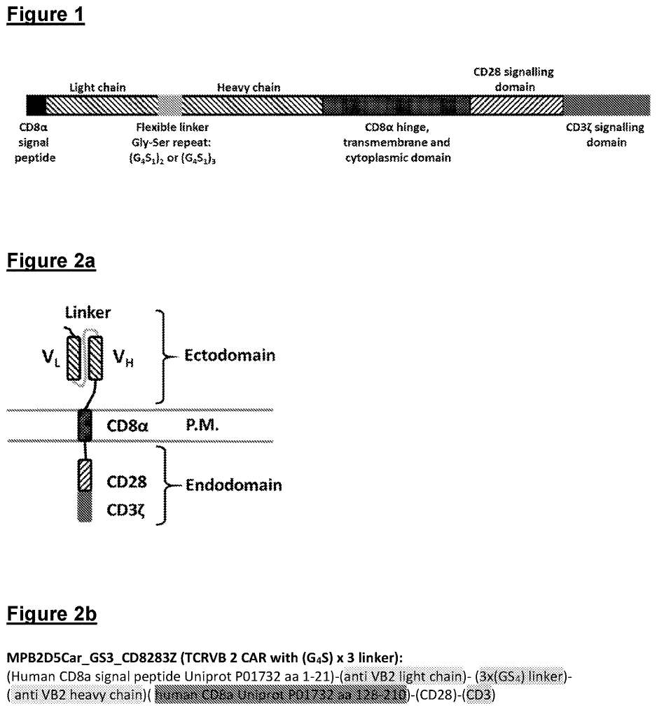

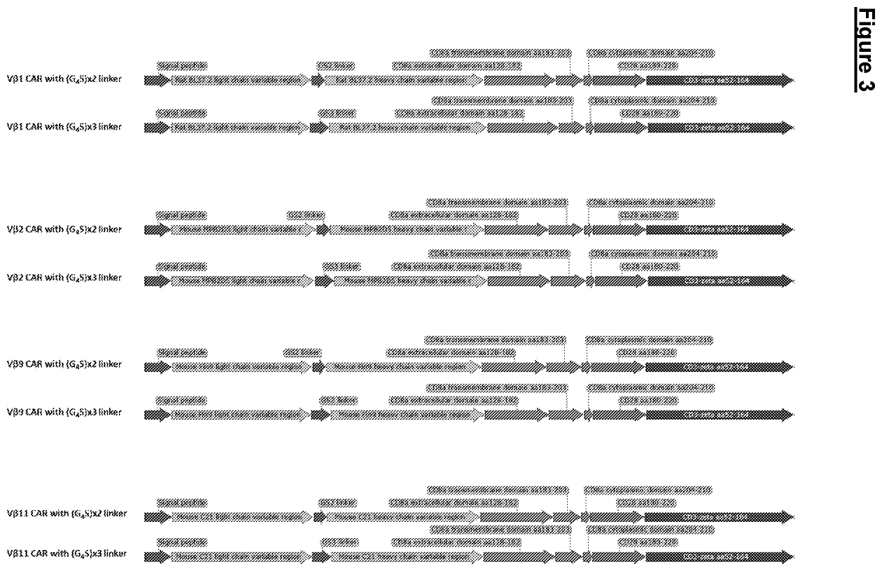

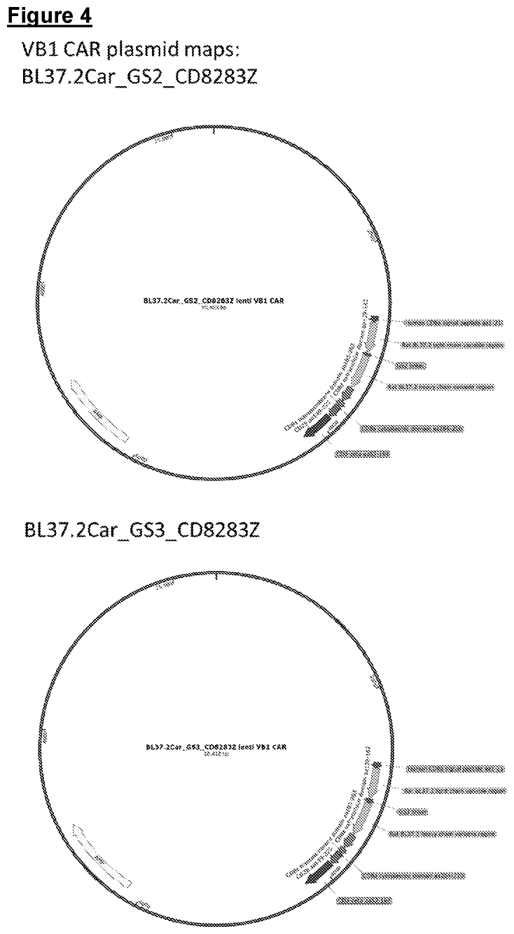

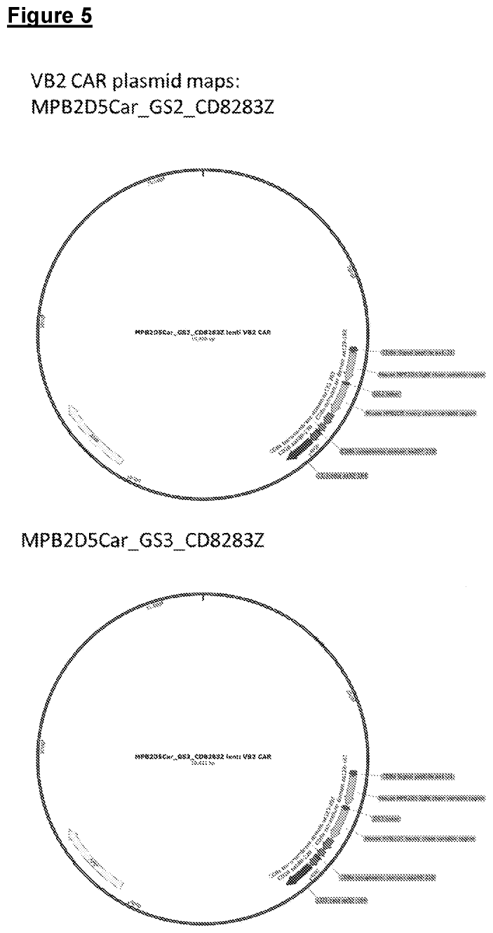

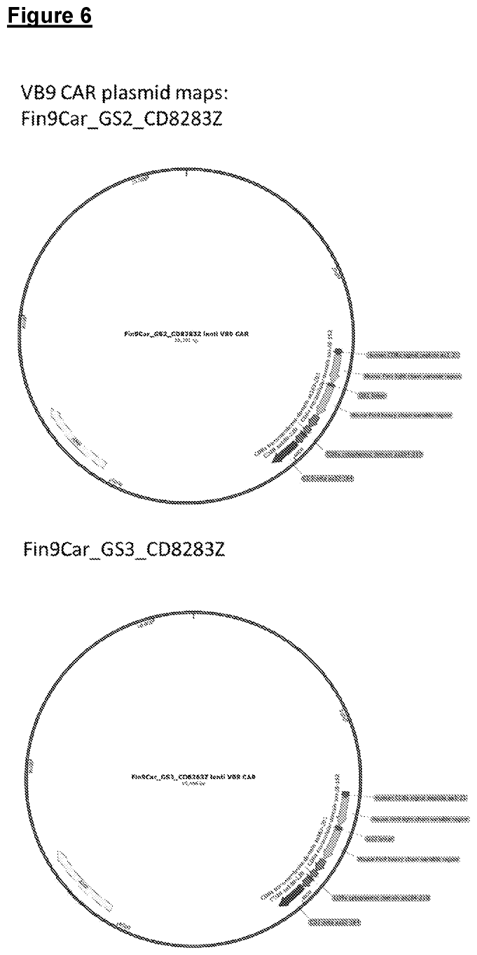

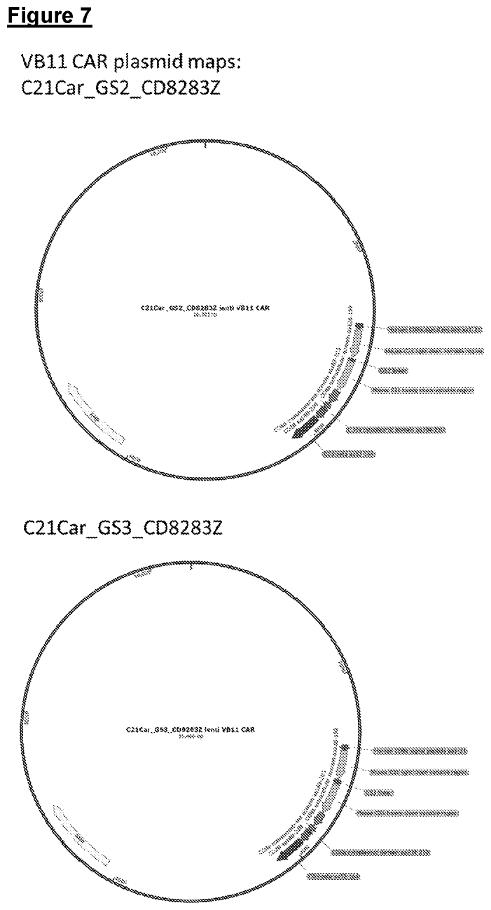

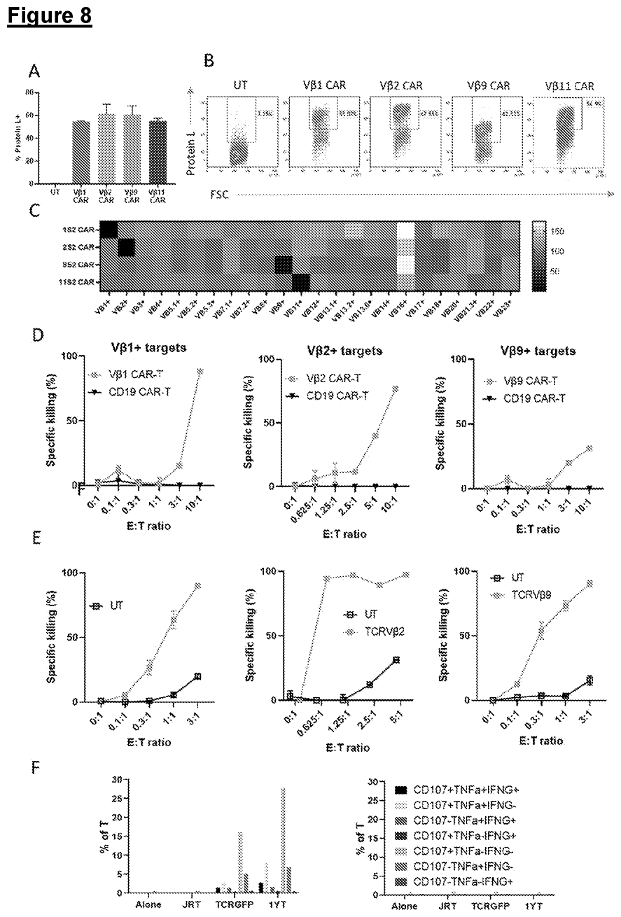

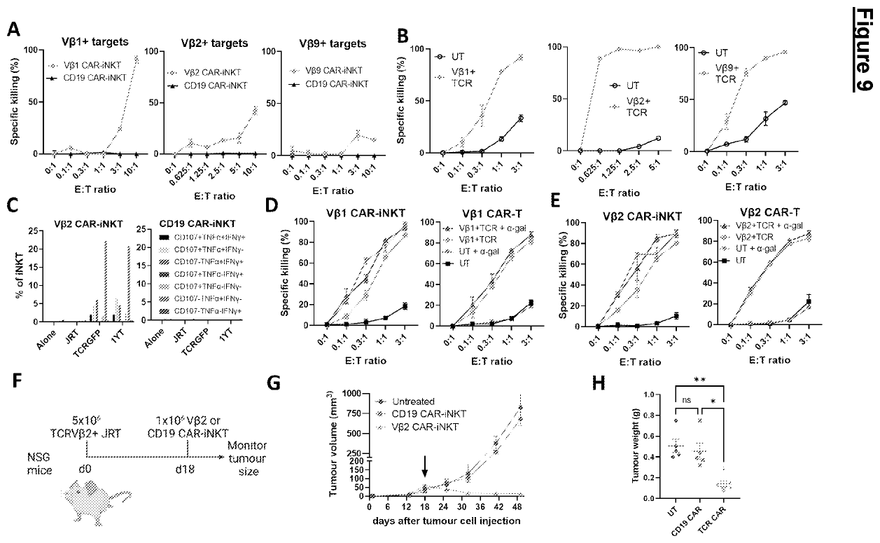

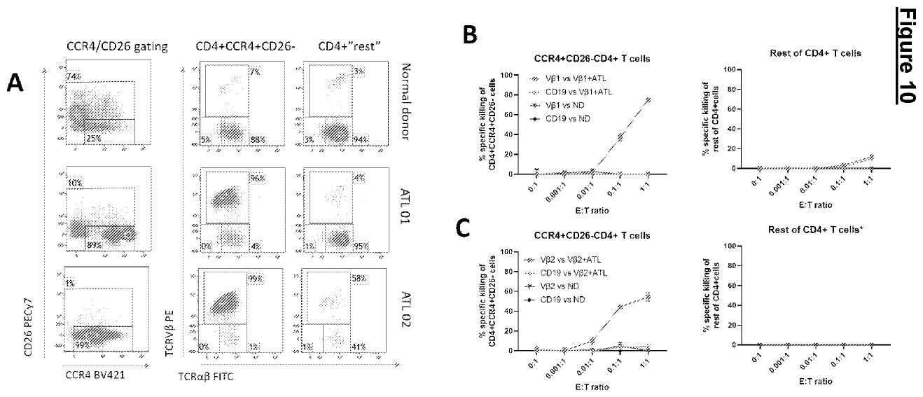

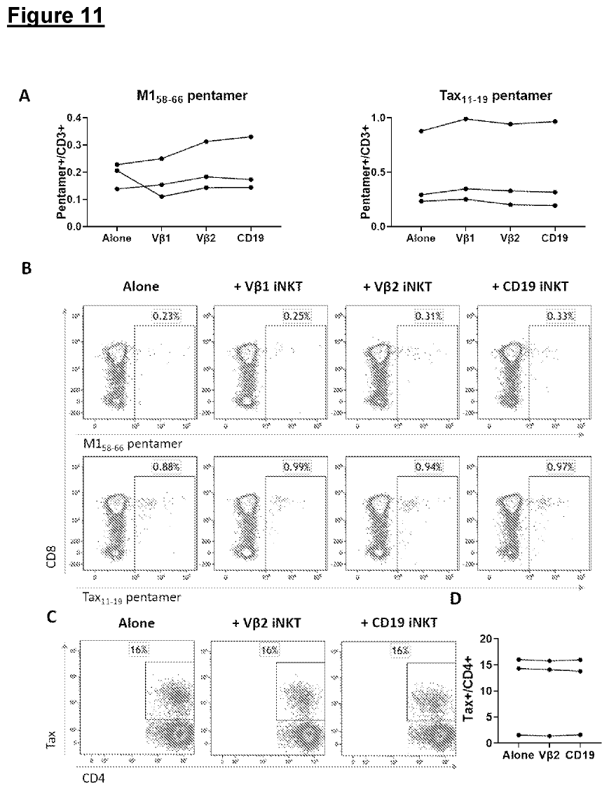

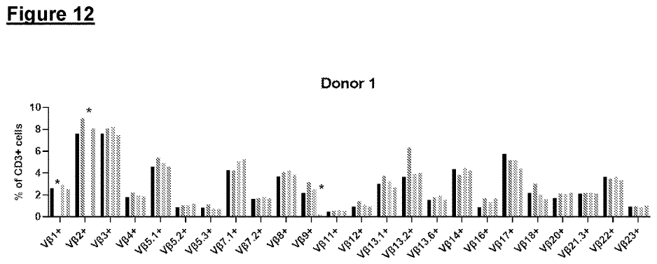

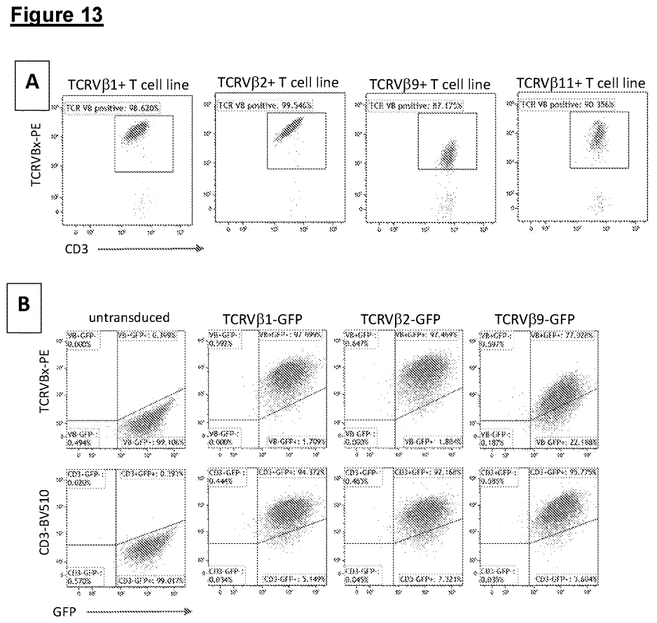

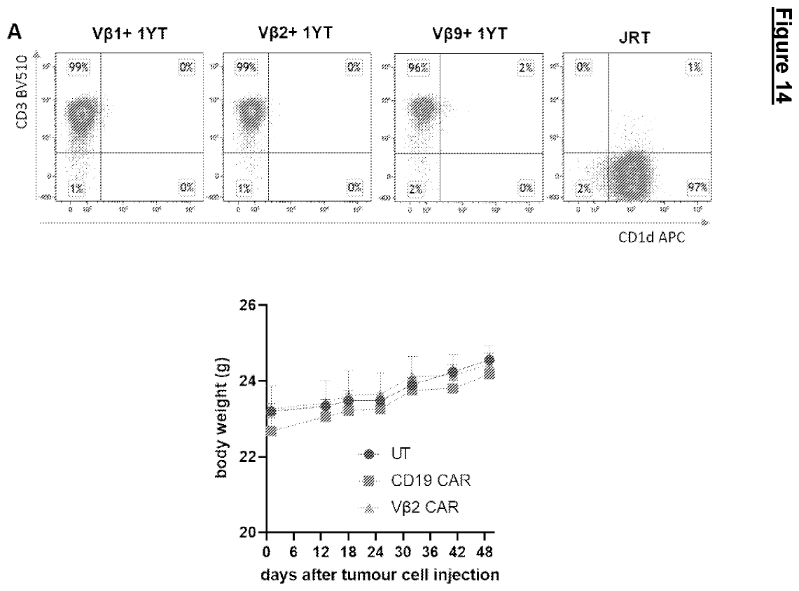

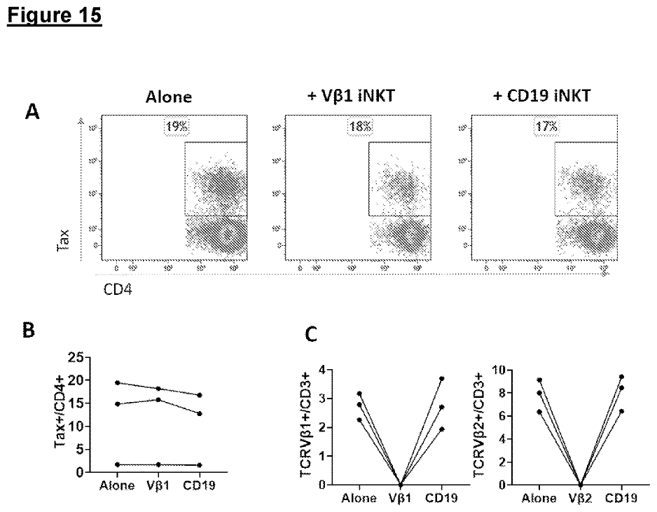

As shown in FIGS. 1 and 2, the CAR construct may comprise a scFv, which may comprise a VL (variable light chain) sequence and a VH (variable heavy chain) sequence. Preferably, the VL sequence is upstream (i.e. 5′ or N-term) of the VH sequence. In some embodiments, however, the VH sequence may be upstream of the VL sequence. Preferably, the VH and VL encoding sequences, in either orientation, are separated by a linker sequence, such as a G4S linker sequence. Preferably, the linker sequence is flexible.

### TCRVβ1

In one preferred embodiment, the CAR construct may comprise a scFv comprising a VL and/or a VH from an anti-TCRVβ1 antibody. The CAR construct preferably comprises a scFv comprising a VL and a VH from an anti-TCRVβ1 antibody. The VL and VH sequences may, in one embodiment, be derived from BL37.2 (i.e. the hybridoma clone name of an anti-TCRVβ1 monoclonal antibody) and comprise a light chain variable region and a heavy chain variable region for binding the TCRVβ1 antigen.

In one embodiment, the light chain of the scFv of the anti-TCRVβ1 antibody can have an amino acid sequence referred to herein as SEQ ID No:7, as follows:

[SEQ ID No: 7]
DVQMTQSPYNLAASPGESVSINCKASKSINKYLAWYQQKPGKPNKLLIYD
GSTLQSGIPSRFSGSGSGTDFTLTIRGLEPEDFGLYYCQQHNEYPPTFGA
GTKLELK

Preferably, therefore, the CAR construct comprises a VL chain having an amino acid sequence substantially as set out in SEQ ID No:7, or a fragment or variant thereof.

In one embodiment, the nucleotide sequence encoding the VL of the scFv of the anti-TCRVβ1 antibody is referred to herein as SEQ ID No:8, as follows:

[SEQ ID No: 8]
GACGTGCAGATGACCCAGAGCCCCTACAACCTGGCCGCCAGCCCCGGCGA
GAGCGTGAGCATCAACTGCAAGGCCAGCAAGAGCATCAACAAGTACCTGG
CCTGGTACCAGCAGAAGCCCGGCAAGCCCAACAAGCTGCTGATCTACGAC
GGCAGCACCCTGCAGAGCGGCATCCCCAGCAGGTTCAGCGGCAGCGGCAG
CGGCACCGACTTCACCCTGACCATCAGGGGCCTGGAGCCCGAGGACTTCG
GCCTGTACTACTGCCAGCAGCACAACGAGTACCCCCCCACCTTCGGCGCC
GGCACCAAGCTGGAGCTGAAG

Preferably, therefore, the VL chain is encoded by a nucleotide sequence substantially as set out in SEQ ID No: 8, or a fragment or variant thereof.

In one embodiment, the heavy chain of the scFv of the anti-TCRVβ1 antibody can have an amino acid sequence referred to herein as SEQ ID No:9, as follows:

[SEQ ID No: 9]
QLQLVQSGPELREPGESVKISCKASGYTFTDYIVHWVKQAPGKGLKWMGW
INTYTGTPTYADDFEGRFVFSLEASASTANLQISNLKNEDTATYFCARSW
RRGIRGIGFDYWGQGVMVTVSS

Preferably, therefore, the CAR construct comprises a VH chain having an amino acid sequence substantially as set out in SEQ ID No:10, or a fragment or variant thereof.

In one embodiment, the nucleotide sequence encoding the VH of the scFv of the anti-TCRVβ1 antibody is referred to herein as SEQ ID No:10, as follows:

[SEQ ID No: 10]
CAGCTGCAGCTGGTGCAGAGCGGCCCCGAGCTGAGGGAGCCCGGCGAGAG
CGTGAAGATCAGCTGCAAGGCCAGCGGCTACACCTTCACCGACTACATCG
TGCACTGGGTGAAGCAGGCCCCCGGCAAGGGCCTGAAGTGGATGGGCTGG
ATCAACACCTACACCGGCACCCCCACCTACGCCGACGACTTCGAGGGCAG
GTTCGTGTTCAGCCTGGAGGCCAGCGCCAGCACCGCCAACCTGCAGATCA
GCAACCTGAAGAACGAGGACACCGCCACCTACTTCTGCGCCAGGAGCTGG
AGGAGGGGCATCAGGGGCATCGGCTTCGACTACTGGGGCCAGGGCGTGAT
GGTGACCGTGAGCAGC

Preferably, therefore, the VH chain is encoded by a nucleotide sequence substantially as set out in SEQ ID No: 10, or a fragment or variant thereof.

### TCRVβ2

In another preferred embodiment, the CAR construct may comprise a scFv comprising a VL and/or a VH from an anti-TCRVβ2 antibody. The CAR construct preferably comprises a scFv comprising a VL and a VH from an anti-TCRVβ2 antibody. The VL and VH sequences may, in one embodiment, be derived from MPB2D5 (i.e. the hybridoma clone name of an anti-TCRVβ2 monoclonal antibody) and comprise a light chain variable region and a heavy chain variable region for binding the TCRVβ2 antigen.

In one embodiment, the light chain of the scFv of the anti-TCRVβ2 antibody can have an amino acid sequence referred to herein as SEQ ID No:1, as follows:

[SEQ ID No: 11]
DIVLTQSPASLAVSLGQRATISCRASKSVSILGTHLIHWYQQKPGQPPKLL
IYAASNLESGVPARFSGSGSETVFTLNIHPVEEEDAATYFCQQSIEDPWTF
GGGTKLGIK

Preferably, therefore, the CAR construct comprises a VL chain having an amino acid sequence substantially as set out in SEQ ID No:11, or a fragment or variant thereof.

In one embodiment, the nucleotide sequence encoding the VL of the scFv of the anti-TCRVβ2 antibody is referred to herein as SEQ ID No:12, as follows:

[SEQ ID No: 12]
GACATCGTGCTGACCCAGAGCCCCGCCAGCCTGGCCGTGAGCCTGGGCCAG
AGGGCCACCATCAGCTGCAGGGCCAGCAAGAGCGTGAGCATCCTGGGCACC
CACCTGATCCACTGGTACCAGCAGAAGCCCGGCCAGCCCCCCAAGCTGCTG
ATCTACGCCGCtAGCAACCTGGAGAGCGGCGTGCCCGCCAGGTTCAGCGGC
AGCGGCAGCGAGACCGTGTTCACCCTGAACATCCACCCCGTGGAGGAGGAG
GACGCCGCCACCTACTTCTGCCAGCAGAGCATCGAGGACCCCTGGACCTTC
GGCGGCGGCACCAAGCTGGGCATCAAG

Preferably, therefore, the VL chain is encoded by a nucleotide sequence substantially as set out in SEQ ID No: 12, or a fragment or variant thereof.

In one embodiment, the heavy chain of the scFv of the anti-TCRVβ2 antibody can have an amino acid sequence referred to herein as SEQ ID No:13, as follows:

[SEQ ID No: 13]
EVQLQQSVADLVRPGASLKLSCTASGFNIKSAYMHWVIQRPDQGPECLGRI
DPATGKTKYAPKFQAKATITADTSSNTAYLQLSSLTSEDTAIYYCTRSLNW
DYGLDYWGQGTSVTVSS

Preferably, therefore, the CAR construct comprises a VH chain having an amino acid sequence substantially as set out in SEQ ID No:13, or a fragment or variant thereof.

In one embodiment, the nucleotide sequence encoding the VH of the scFv of the anti-TCRVβ1 antibody is referred to herein as SEQ ID No:14, as follows:

[SEQ ID No: 14]
GAGGTGCAGCTGCAGCAGAGCGTGGCCGACCTGGTGAGGCCCGGCGCCAGC
CTGAAGCTGAGCTGCACCGCCAGCGGCTTCAACATCAAGAGCGCCTACATG
CACTGGGTGATCCAGAGGCCCGACCAGGGCCCCGAGTGCCTGGGCAGGATC
GACCCCGCCACCGGCAAGACCAAGTACGCCCCCAAGTTCCAGGCCAAGGCC
ACCATCACCGCCGACACCAGCAGCAACACCGCCTACCTGCAGCTGAGCAGC
CTGACCAGCGAGGACACCGCCATCTACTACTGCACCAGGAGCCTGAACTGG
GACTACGGCCTGGACTACTGGGGCCAGGGCACCAGCGTGACCGTGAGCAGC

Preferably, therefore, the VH chain is encoded by a nucleotide sequence substantially as set out in SEQ ID No: 14, or a fragment or variant thereof.

### TCRVβ9

In yet another preferred embodiment, the CAR construct may comprise a scFv comprising a VL and/or a VH from an anti-TCRVβ9 antibody. The CAR construct preferably comprises a scFv comprising a VL and a VH from an anti-TCRVβ9 antibody. The VL and VH sequences may, in one embodiment, be derived from FIN9 (i.e. the hybridoma clone name of an anti-TCRVβ9 monoclonal antibody) and comprise a light chain variable region and a heavy chain variable region for binding the TCRVβ9 antigen.

In one embodiment, the light chain of the scFv of the anti-TCRVβ9 antibody can have an amino acid sequence referred to herein as SEQ ID No:15, as follows:

[SEQ ID No: 15]
ETTVTQSPASLSVATGEKVTIRCISSTDIDDDMNWYQQKSGEPPKLLISEG
NTLRPGVPSRFSSSGYGTDFVFTIENMLSEDVADYYCLQSDNMPLTFGAGT
KLELK

Preferably, therefore, the CAR construct comprises a VL chain having an amino acid sequence substantially as set out in SEQ ID No:15, or a fragment or variant thereof.

In one embodiment, the nucleotide sequence encoding the VL of the scFv of the anti-TCRVβ9 antibody is referred to herein as SEQ ID No:16, as follows:

[SEQ ID No: 16]
GAGACCACCGTGACCCAGAGCCCCGCCAGCCTGAGCGTGGCCACCGGCGAG
AAGGTGACCATCAGGTGCATCAGCAGCACCGACATCGACGACGACATGAAC
TGGTACCAGCAGAAGAGCGGCGAGCCCCCCAAGCTGCTGATCAGCGAGGGC
AACACCCTGAGGCCCGGCGTGCCCAGCAGGTTCAGCAGCAGCGGCTACGGC
ACCGACTTCGTGTTCACCATCGAGAACATGCTGAGCGAGGACGTGGCCGAC
TACTACTGCCTGCAGAGCGACAACATGCCCCTGACCTTCGGCGCCGGCACC
AAGCTGGAGCTGAAG

Preferably, therefore, the VL chain is encoded by a nucleotide sequence substantially as set out in SEQ ID No: 16, or a fragment or variant thereof.

In one embodiment, the heavy chain of the scFv of the anti-TCRVβ9 antibody can have an amino acid sequence referred to herein as SEQ ID No:17, as follows:

[SEQ ID No: 17]
EVQLQQSVAELVRPGASVKLSCTASGFNIKNTFMHWVKQRPEQGLEWIGRI
DPTNGYTKFAPKFQGKATLTAVTSSNTVYLQLSSLTSEDTAIYYCAHDYDA
PWFAYWGQGTLVIVSA

Preferably, therefore, the CAR construct comprises a VH chain having an amino acid sequence substantially as set out in SEQ ID No:17, or a fragment or variant thereof.

In one embodiment, the nucleotide sequence encoding the VH of the scFv of the anti-TCRVβ9 antibody is referred to herein as SEQ ID No:18, as follows:

[SEQ ID No: 18]
GAGGTGCAGCTGCAGCAGAGCGTGGCCGAGCTGGTGAGGCCCGGCGCCAGC
GTGAAGCTGAGCTGCACCGCCAGCGGCTTCAACATCAAGAACACCTTCATG
CACTGGGTGAAGCAGAGGCCCGAGCAGGGCCTGGAGTGGATCGGCAGGATC
GACCCCACCAACGGCTACACCAAGTTCGCCCCCAAGTTCCAGGGCAAGGCC
ACCCTGACCGCCGTGACCAGCAGCAACACCGTGTACCTGCAGCTGAGCAGC
CTGACCAGCGAGGACACCGCCATCTACTACTGCGCCCACGACTACGACGCC
CCCTGGTTCGCCTACTGGGGCCAGGGCACCCTGGTGATCGTGAGCGCC

Preferably, therefore, the VH chain is encoded by a nucleotide sequence substantially as set out in SEQ ID No: 18, or a fragment or variant thereof.

### TCRVβ11

In yet another preferred embodiment, the CAR construct may comprise a scFv comprising a VL and/or a VH from an anti-TCRVβ11 antibody. The CAR construct preferably comprises a scFv comprising a VL and a VH from an anti-TCRVβ11 antibody. The VL and VH sequences may, in one embodiment, be derived from C21 (i.e. the hybridoma clone name of an anti-TCRVβ11 monoclonal antibody) and comprise a light chain variable region and a heavy chain variable region for binding the TCRVβ11 antigen.

In one embodiment, the light chain of the scFv of the anti-TCRVβ11 antibody can have an amino acid sequence referred to herein as SEQ ID No:19, as follows:

[SEQ ID No: 19]
DIKMTQSPSSMYASLGERVTITCKASQDINSYLSWFQQKAGKSPKTLIYRA
NRLVDGVPSRFSGSGSGQDYSLTISSLEYEDMGIYYCLQYDEFPFTFGGGT
RLEIK

Preferably, therefore, the CAR construct comprises a VL chain having an amino acid sequence substantially as set out in SEQ ID No:19, or a fragment or variant thereof.

In one embodiment, the nucleotide sequence encoding the VL chain of the scFv of the anti-TCRVβ11 antibody is referred to herein as SEQ ID No:20, as follows:

[SEQ ID No: 20]
GACATTAAGATGACCCAGTCCCCCTCCTCCATGTATGCCAGCCTCGGCGAG
AGAGTCACCATCACATGCAAGGCCAGCCAAGACATCAACAGCTACCTCAGC
TGGTTCCAGCAGAAAGCCGGCAAGAGCCCCAAGACACTGATCTATAGGGCT
AATAGACTGGTGGACGGCGTGCCTAGCAGATTTTCCGGCAGCGGCAGCGGC
CAAGACTATTCTCTGACCATCAGCTCTCTGGAGTACGAGGACATGGGAATC
TACTACTGTCTGCAGTACGACGAGTTCCCCTTCACCTTCGGAGGCGGCACA
AGACTGGAAATCAAA

Preferably, therefore, the VL chain is encoded by a nucleotide sequence substantially as set out in SEQ ID No: 20, or a fragment or variant thereof.

In one embodiment, the heavy chain of the scFv of the anti-TCRVβ11 antibody can have an amino acid sequence referred to herein as SEQ ID No:21, as follows:

[SEQ ID No: 21]
QVQLQQSGPEVVRPGVSVKISCKGSGYRFTDSAMHWVKQSHAKSLEWIGVI
SSYNGNTNYNQKFKGKATMTVDKSSSTAYMELARMTSEDSAIYYCARSRDA
MDYWGQGTSVTVSS

Preferably, therefore, the CAR construct comprises a VH chain having an amino acid sequence substantially as set out in SEQ ID No:21, or a fragment or variant thereof.

In one embodiment, the nucleotide sequence encoding the VH of the scFv of the anti-TCRVβ11 antibody is referred to herein as SEQ ID No:22, as follows:

[SEQ ID No: 22]
CAAGTGCAGCTCCAGCAGTCCGGACCCGAGGTGGTGAGGCCCGGCGTGAGC
GTGAAGATCAGCTGCAAGGGCAGCGGCTATAGGTTCACCGACTCCGCCATG
CACTGGGTGAAGCAGTCCCATGCCAAGAGCCTCGAGTGGATCGGCGTGATC
AGCAGCTACAACGGCAACACCAACTACAACCAGAAGTTCAAGGGCAAGGCC
ACAATGACCGTGGACAAGAGCAGCTCCACCGCCTACATGGAGCTGGCCAGA
ATGACCAGCGAGGATAGCGCCATCTACTACTGTGCTAGGTCTAGAGACGCC
ATGGACTACTGGGGCCAAGGCACATCCGTGACCGTGAGCTCC

Preferably, therefore, the VH chain is encoded by a nucleotide sequence substantially as set out in SEQ ID No: 22, or a fragment or variant thereof.

Preferably, the VH (e.g. SEQ ID No: 9, 13, 17 or 21) and VL (e.g. SEQ ID No: 7, 11, 15 or 19) sequences, when in either orientation, are separated by a linker sequence. In an embodiment, the linker sequence may comprise at least one a G4S linker sequence, which may be referred to herein as SEQ ID No:23, as follows:

[SEQ ID No: 23]
GGGGS

Preferably, therefore, the CAR construct comprises a linker sequence comprising an amino acid sequence substantially as set out in SEQ ID No:23, or a fragment or variant thereof.

In other embodiments, the linker sequence may comprise a plurality of repeats of the G4S linker sequence. For example, the linker sequence may comprise two or three repeats of the G4S linker sequence (i.e. 2×G₄S, or 3×G₄S).

The CAR construct preferably comprises a hinge domain. Preferably, the hinge domain comprises: (i) an extracellular domain, or a portion thereof; (ii) a transmembrane (TM) domain, or a portion thereof; and/or (iii) a cytoplasmic domain, or a portion thereof. Most preferably, the hinge domain comprises: (i) an extracellular domain, or a portion thereof; (ii) a transmembrane (TM) domain, or a portion thereof; and (iii) a cytoplasmic domain, or a portion thereof. Advantageously, the hinge domain is configured for CAR display and anchoring on the CAR-T cell. Preferably, the hinge domain is disposed C-term of the antigen-binding domain, more preferably the VH chain.

Preferably, the hinge domain comprises a CD8α sequence, or a portion thereof, and more preferably a human CD8α sequence, or a portion thereof. Advantageously, a longer CD8α-derived hinge as compared to a shorter CD8α-derived hinge significantly decreases the toxicity of the CAR construct. Preferably, therefore, in one embodiment, the hinge domain comprises or consists of substantially the full length of human CD8α, or a portion thereof.

In one embodiment, the amino acid sequence of human CD8α is referred to herein as SEQ ID No: 51, as follows:

[SEQ ID No: 51]
MALPVTALLLPLALLLHAARPSQFRVSPLDRTWNLGETVELKCQVLLSNP
TSGCSWLFQPRGAAASPTFLLYLSQNKPKAAEGLDTQRFSGKRLGDTFVL
TLSDFRRENEGYYFCSALSNSIMYFSHFVPVFLPAKPTTTPAPRPPTPAP
TIASQPLSLRPEACRPAAGGAVHTRGLDFACDIYIWAPLAGTCGVLLLSL
VITLYCNHRNRRRVCKCPRPVVKSGDKPSLSARYV

Preferably, the CAR construct comprises or consists of a hinge domain that is derived from human CD8α comprising at least a portion of the extracellular domain. In one embodiment, the amino acid sequence of a human CD8α extracellular domain or a portion thereof is referred to herein as SEQ ID No: 24, as follows:

[SEQ ID No: 24]
FVPVFLPAKPTTTPAPRPPTPAPTIASQPLSLRPEACRPAAGGAVHTRGL
DFACD

Preferably, therefore, the CAR construct comprises a hinge domain comprising an amino acid sequence substantially as set out in SEQ ID No: 24, or a fragment or variant thereof.

Preferably, the CAR construct comprises or consists of a hinge domain that is derived from human CD8α comprising a full transmembrane helical domain. In one embodiment, the amino acid sequence of a human CD8α full transmembrane domain is referred to herein as SEQ ID No:49, as follows:

[SEQ ID No: 49]
IYIWAPLAGTCGVLLLSLVIT

Preferably, therefore, the CAR construct comprises or consists of a hinge domain comprising an amino acid sequence substantially as set out in SEQ ID No:49, or a fragment or variant thereof.

Preferably, the CAR construct comprises or consists of a hinge domain that is derived from human CD8α comprising at least a portion of the cytoplasmic domain. In one embodiment, the amino acid sequence of a human CD8α cytoplasmic domain or a portion thereof is referred to herein as SEQ ID No:50, as follows:

[SEQ ID No: 50]
LYCNHRN

Preferably, therefore, the CAR construct comprises or consists of a hinge domain comprising an amino acid sequence substantially as set out in SEQ ID No:50, or a fragment or variant thereof.

In a preferred embodiment, the CAR construct comprises a hinge domain that is derived from human CD8α. Preferably, the hinge comprises or consists of amino acids 128-182 of human CD8α, as represented by SEQ ID No: 51, i.e. the extracellular domain. Preferably, the hinge comprising a portion of human CD8α extracellular domain, human CD8α full transmembrane domain, and a portion of human CD8α cytoplasmic domain is defined by amino acids 128-210 of human CD8α can have an amino acid sequence referred to herein as SEQ ID No:25, as follows:

[SEQ ID No: 25]
FVPVFLPAKPTTTPAPRPPTPAPTIASQPLSLRPEACRPAAGGAVHTRGL
DFACDIYIWAPLAGTCGVLLLSLVITLYCNHRN

Preferably, therefore, the CAR construct comprises or consists of a hinge domain having an amino acid sequence substantially as set out in SEQ ID No:25, or a fragment or variant thereof.

Advantageously, SEQ ID No: 25 comprises 55 amino acids from the extracellular domain (FVPVFLPAKPTTTPAPRPPTPAPTIASQPLSLRPEACRPAAGGAVHTRGLDFACD—SEQ ID No: 24), the full transmembrane helical domain (IYIWAPLAGTCGVLLLSLVIT—SEQ ID No: 49) and 7 amino acids of the cytoplasmic domain (LYCNHRN—SEQ ID No: 50). This configuration advantageously confers to the CAR construct less risk of cytokine release syndrome and neurotoxicity, which are side effects commonly associated with CAR-T therapy, without negatively impacting on the construct's efficacy.

In one embodiment, the CAR construct comprises or consists of a hinge domain that is derived from human CD8α comprising the extracellular domain of human CD8α defined by amino acids 138-182 of human CD8α, as represented by SEQ ID No: 51.

Preferably, the CD8α-derived hinge comprising the extracellular domain of human CD8α is at least one, two or three amino acids longer than amino acids 138-182 of human CD8α on the N-terminus, as represented by SEQ ID No: 51. Therefore, preferably the CD8α-derived hinge comprises the extracellular domain of human CD8α defined by amino acids 137-182, 136-182, or 135-182 of human CD8α, as represented by SEQ ID No: 51.

More preferably, however, the CD8α-derived hinge comprising the extracellular domain of human CD8α is at least four, five or six amino acids longer amino acids 138-182 of human CD8α on the N-terminus, as represented by SEQ ID No: 51. Therefore, more preferably the CD8α-derived hinge comprises the extracellular domain of human CD8α defined by amino acids 134-182, 133-182, or 132-182 of human CD8α, as represented by SEQ ID No: 51.

Most preferably, the CD8α-derived hinge comprising the extracellular domain of human CD8α is at least seven, eight, nine or ten amino acids longer amino acids 138-182 of human CD8α on the N-terminus, as represented by SEQ ID No: 51. Therefore, most preferably the CD8α-derived hinge comprises the extracellular domain of human CD8α defined by amino acids 131-182, 130-182, 129-182 or 128-182 of human CD8α, as represented by SEQ ID No: 51.

In one embodiment, the CAR construct comprises or consists of a hinge domain that is derived from human CD8α comprising the transmembrane domain of human CD8α defined by amino acids 183-203 of human CD8α, as represented by SEQ ID No: 51.

In another embodiment, the CAR construct comprises or consists of a hinge domain that is derived from human CD8α comprising the cytoplasmic domain of human CD8α defined by amino acids 204-206 of human CD8α.

Preferably, the CD8α-derived hinge comprising the cytoplasmic domain of human CD8α is at least one amino acid longer than amino acids 204-206 of human CD8α, as represented by SEQ ID No: 51 on the C-terminus. Therefore, preferably the CD8α-derived hinge comprises the cytoplasmic domain of human CD8α defined by amino acids 204-207 of human CD8α, as represented by SEQ ID No: 51.

More preferably, the CD8α-derived hinge comprising the cytoplasmic domain of human CD8α is at least two amino acids longer than amino acids 204-206 of human CD8α as represented by SEQ ID No: 51 on the C-terminus. Therefore, more preferably the CD8α-derived hinge comprises the cytoplasmic domain of human CD8α defined by amino acids 204-208 of human CD8α, as represented by SEQ ID No: 51.

Even more preferably, the CD8α-derived hinge comprising the cytoplasmic domain of human CD8α is at least three amino acids longer than amino acids 204-206 of human CD8α as represented by SEQ ID No: 51 on the C-terminus.

Therefore, even more preferably, the CD8α-derived hinge comprises the cytoplasmic domain of human CD8α defined by amino acids 204-209 of human CD8α, as represented by SEQ ID No: 51.

Most preferably, the CD8α-derived hinge comprising the cytoplasmic domain of human CD8α is at least four amino acids longer than amino acids 204-206 of human CD8α, as represented by SEQ ID No: 51 on the C-terminus. Therefore, most preferably the CD8α-derived hinge comprises the cytoplasmic domain of human CD8α defined by amino acids 204-210 of human CD8α, as represented by SEQ ID No: 51.

In one embodiment, a nucleotide sequence encoding the hinge domain is referred to herein as SEQ ID No:26, as follows:

[SEQ ID No: 26]
ttcgtgcctgtgtttctgcctgccaagcccaccacaacccctgcccctag
acctcctacacccgcccctacaatcgccagccagcctctgtctctgaggc
ccgaggcttgtagacctgctgctggcggagccgtgcacaccagaggactg
gatttcgcctgcgacatctacatctgggcccctctggccggcacatgtgg
cgtgctgctgctgagcctcgtgatcaccctgtactgcaaccaccggaac

Preferably, therefore, the construct comprises a hinge domain encoded by a nucleotide sequence substantially as set out in SEQ ID No: 26, or a fragment or variant thereof.

The CAR construct preferably comprises an intracellular domain, which comprises a primary stimulatory (or signalling) CD3ζ chain and/or a co-stimulatory (or signalling) domain of CD28, and more preferably a co-stimulatory/signalling domain of CD28 and a stimulatory CD3ζ chain. It will be appreciated that these components form the basis of a second generation CAR and are required for triggering the intracellular signalling pathway. Preferably, the intracellular domain is disposed 3′ of the sequence encoding the hinge domain. The co-stimulatory domain of CD28 may be N-terminus of the CD3ζ chain. Advantageously, CD28 co-stimulatory domain significantly improved the efficacy of CD19 CAR T cells in patients with B cell lymphoma and B cell acute leukaemia and provides TCRVβ CARs with significant efficacy against T cell lymphoma and leukaemia in vitro and in vivo.

In another embodiment, the CAR construct may comprise one or two co-stimulatory domains, which may be selected from CD28, 4-1BB signalling domain and OX40 signalling domain.

One embodiment of the 4-1BB signalling domain can have an amino acid sequence, which is referred to herein as SEQ ID No: 46, as follows:

[SEQ ID No: 46]
RFSVVKRGRKKLLYIFKQPFMRPVQTTQEEDGCSCRFPEEEEGGCEL

Preferably, therefore, the construct comprises a nucleotide sequence encoding an amino acid sequence substantially as set out in SEQ ID No: 46, or a fragment or variant thereof.

In an embodiment, the 4-1BB signalling domain can be encoded by a nucleic acid sequence, which is referred to herein as SEQ ID No: 47, as follows:

[SEQ ID No: 47]
CGTTTCTCTGTTGTTAAACGGGGCAGAAAGAAGCTCCTGTATATATTCAA
ACAACCATTTATGAGACCAGTACAAACTACTCAAGAGGAAGATGGCTGTA
GCTGCCGATTTCCAGAAGAAGAAGAAGGAGGATGTGAACTG

Preferably, therefore, the construct comprises a nucleotide sequence substantially as set out in SEQ ID No: 47, or a fragment or variant thereof.

Therefore, in one embodiment, the co-stimulatory domain of CD28 is referred to herein as SEQ ID No:27, as follows:

[SEQ ID No: 27]
RSKRSRLLHSDYMNMTPRRPGPTRKHYQPYAPPRDFAAYRS

Preferably, therefore, the CAR construct comprises a co-stimulatory domain of CD28 having an amino acid sequence substantially as set out in SEQ ID No:27, or a fragment or variant thereof.

In one embodiment, a nucleotide sequence encoding the co-stimulatory domain of CD28 is referred to herein as SEQ ID No:28, as follows:

[SEQ ID No: 28]
agaagcaagcggagccggctgctgcacagcgactacatgaacatgacccc
cactaccagccttacgcccctcccagagacttcgccgcctaccggtcc

Preferably, therefore, the co-stimulatory domain of CD28 is encoded by a nucleotide sequence substantially as set out in SEQ ID No: 28, or a fragment or variant thereof.

In a preferred embodiment, the CAR construct further comprises the stimulatory protein CD3ζ. In an embodiment, the stimulatory protein CD3ζ can have an amino acid sequence referred to herein as SEQ ID No:29, as follows:

[SEQ ID No: 29]
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

Preferably, therefore, the CAR construct comprises a stimulatory protein CD3ζ having an amino acid sequence substantially as set out in SEQ ID No: 29, or a fragment or variant thereof.

In one embodiment, a nucleotide sequence encoding the stimulatory protein CD3ζ is referred to herein as SEQ ID No:30, as follows:

[SEQ ID No: 30]
agagtgaagttcagcagaagcgccgacgcccctgcctatcagcagggcca
gaaccagctgtacaacgagctgaacctgggcagacgggaagagtacgatg
tgctggacaaaagacgtggccgggaccctgagatggggggaaagccgaga
aggaagaaccctcaggaaggcctgtacaatgaactgcagaaagataagat
ggcggaggcctacagtgagattgggatgaaaggcgagcgccggaggggca
aggggcacgatggcctttaccagggtctcagtacagccaccaaggacacc
tacgacgcccttcacatgcaggccctgccccctcgc

Preferably, therefore, the stimulatory protein CD3ζ is encoded by a nucleotide sequence substantially as set out in SEQ ID No: 30, or a fragment or variant thereof.

Accordingly, in a preferred embodiment, the intracellular domain of the CAR construct of the first aspect comprises both co-stimulating domain of CD28 and the CD3ζ stimulatory domain.

It will be appreciated that the position of each of the components in the CAR construct described herein is interchangeable. FIG. 1 provides a schematic map of an exemplary embodiment of the anti-TCRVβ CAR construct.

Accordingly, in a preferred embodiment, the CAR construct comprises each of the elements in the following order: a 5′/N-term variable light chain of an anti-TCRVβ antibody-a variable heavy chain of an anti-TCRVβ antibody-a hinge-a co-stimulatory domain comprising CD28-3′/C-term stimulatory protein CD3ζ. The use of N- and C-term (and also 5′ and 3′) indicates that the features are either upstream or downstream in the construct (protein or DNA, respectively), and is not intended to indicate that the features are necessarily terminal features.

More preferably, the CAR construct comprises a 5′/N-term signal peptide-a variable light chain of an anti-TCRVβ antibody-a linker-a variable heavy chain of an anti-TCRVβ antibody-a hinge-a co-stimulatory domain comprising CD28-3′/C-term stimulatory protein CD3ζ.

### “BL37.2 CAR”

In one preferred embodiment, the anti-TCRVβ CAR construct is an anti-TCRVβ1 CAR (known herein as “BL37.2 CAR”). Preferably, the anti-TCRVβ1 CAR comprises a 2×G₄S linker. In an embodiment, the anti-TCRVβ1 CAR (having a 2×G₄S linker) has an amino acid sequence referred to herein as SEQ ID No:31, as follows:

[SEQ ID No: 31]
MALPVTALLLPLALLLHAARPDVQMTQSPYNLAASPGESVSINCKASKSI
NKYLAWYQQKPGKPNKLLIYDGSTLQSGIPSRFSGSGSGTDFTLTIRGLE
PEDFGLYYCQQHNEYPPTFGAGTKLELKGGGGSGGGGSQLQLVQSGPELR
EPGESVKISCKASGYTFTDYIVHWVKQAPGKGLKWMGWINTYTGTPTYAD
DFEGRFVFSLEASASTANLQISNLKNEDTATYFCARSWRRGIRGIGFDYW
GQGVMVTVSSFVPVFLPAKPTTTPAPRPPTPAPTIASQPLSLRPEACRPA
AGGAVHTRGLDFACDIYIWAPLAGTCGVLLLSLVITLYCNHRNRSKRSRL
LHSDYMNMTPRRPGPTRKHYQPYAPPRDFAAYRSRVKFSRSADAPAYQQG
QNQLYNELNLGRREEYDVLDKRRGRDPEMGGKPRRKNPQEGLYNELQKDK
MAEAYSEIGMKGERRRGKGHDGLYQGLSTATKDTYDALHMQALPPR

Preferably, therefore, the CAR construct comprises an amino acid sequence substantially as set out in SEQ ID No:31, or a fragment or variant thereof.

In one embodiment, a nucleotide sequence encoding the anti-TCRVβ1 CAR (having a 2×G₄S linker) is referred to herein as SEQ ID No:32, as follows:

[SEQ ID No: 32]
atggctctgcctgtgacagctctgctgctgcctctggccctgctgctgca
tgccgccagacctGACGTGCAGATGACCCAGAGCCCCTACAACCTGGCCG
CCAGCCCCGGCGAGAGCGTGAGCATCAACTGCAAGGCCAGCAAGAGCATC
AACAAGTACCTGGCCTGGTACCAGCAGAAGCCCGGCAAGCCCAACAAGCT
GCTGATCTACGACGGCAGCACCCTGCAGAGCGGCATCCCCAGCAGGTTCA
GCGGCAGCGGCAGCGGCACCGACTTCACCCTGACCATCAGGGGCCTGGAG
CCCGAGGACTTCGGCCTGTACTACTGCCAGCAGCACAACGAGTACCCCCC
CACCTTCGGCGCCGGCACCAAGCTGGAGCTGAAGGGAGGCGGAGGCTCTG
GCGGAGGCGGCTCTCAGCTGCAGCTGGTGCAGAGCGGCCCCGAGCTGAGG
GAGCCCGGCGAGAGCGTGAAGATCAGCTGCAAGGCCAGCGGCTACACCTT
CACCGACTACATCGTGCACTGGGTGAAGCAGGCCCCCGGCAAGGGCCTGA
AGTGGATGGGCTGGATCAACACCTACACCGGCACCCCCACCTACGCCGAC
GACTTCGAGGGCAGGTTCGTGTTCAGCCTGGAGGCCAGCGCCAGCACCGC
CAACCTGCAGATCAGCAACCTGAAGAACGAGGACACCGCCACCTACTTCT
GCGCCAGGAGCTGGAGGAGGGGCATCAGGGGCATCGGCTTCGACTACTGG
GGCCAGGGCGTGATGGTGACCGTGAGCAGCttcgtgcctgtgtttctgcc
tgccaagcccaccacaacccctgcccctagacctcctacacccgccccta
caatcgccagccagcctctgtctctgaggcccgaggcttgtagacctgct
gctggcggagccgtgcacaccagaggactggatttcgcctgcgacatcta
catctgggcccctctggccggcacatgtggcgtgctgctgctgagcctcg
tgatcaccctgtactgcaaccaccggaacagaagcaagcggagccggctg
ctgcacagcgactacatgaacatgacccccagacggcctggccccaccag
aaagcactaccagccttacgcccctcccagagacttcgccgcctaccggt
ccagagtgaagttcagcagaagcgccgacgcccctgcctatcagcagggc
cagaaccagctgtacaacgagctgaacctgggcagacgggaagagtacga
tgtgctggacaaaagacgtggccgggaccctgagatggggggaaagccga
gaaggaagaaccctcaggaaggcctgtacaatgaactgcagaaagataag
atggcggaggcctacagtgagattgggatgaaaggcgagcgccggagggg
caaggggcacgatggcctttaccagggtctcagtacagccaccaaggaca
cctacgacgcccttcacatgcaggccctgccccctcgc

Preferably, therefore, the CAR construct is encoded by a nucleotide sequence substantially as set out in SEQ ID No: 32, or a fragment or variant thereof.

In another preferred embodiment, the anti-TCRVβ1 CAR construct (known as “BL37.2 CAR”) comprises a 3×G₄S linker. In an embodiment, the anti-TCRVβ1 CAR (having a 3×G₄S linker) can have an amino acid sequence referred to herein as SEQ ID No:33, as follows:

[SEQ ID No: 33]
MALPVTALLLPLALLLHAARPDVQMTQSPYNLAASPGESVSINCKASKSI
NKYLAWYQQKPGKPNKLLIYDGSTLQSGIPSRFSGSGSGTDFTLTIRGLE
PEDFGLYYCQQHNEYPPTFGAGTKLELKGGGGSGGGGSGGGGSQLQLVQS
GPELREPGESVKISCKASGYTFTDYIVHWVKQAPGKGLKWMGWINTYTGT
PTYADDFEGRFVFSLEASASTANLQISNLKNEDTATYFCARSWRRGIRGI
GFDYWGQGVMVTVSSFVPVFLPAKPTTTPAPRPPTPAPTIASQPLSLRPE
ACRPAAGGAVHTRGLDFACDIYIWAPLAGTCGVLLLSLVITLYCNHRNRS
KRSRLLHSDYMNMTPRRPGPTRKHYQPYAPPRDFAAYRSRVKFSRSADAP
AYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPP
R

Preferably, therefore, the CAR construct comprises an amino acid sequence substantially as set out in SEQ ID No:33, or a fragment or variant thereof.

In one embodiment, a nucleotide sequence encoding the anti-TCRVβ1 CAR (with a 3×G₄S linker) is referred to herein as SEQ ID No:34, as follows:

[SEQ ID No: 34]
atggctctgcctgtgacagctctgctgctgcctctggccctgctgctgca
tgccgccagacctGACGTGCAGATGACCCAGAGCCCCTACAACCTGGCCG
CCAGCCCCGGCGAGAGCGTGAGCATCAACTGCAAGGCCAGCAAGAGCATC
AACAAGTACCTGGCCTGGTACCAGCAGAAGCCCGGCAAGCCCAACAAGCT
GCTGATCTACGACGGCAGCACCCTGCAGAGCGGCATCCCCAGCAGGTTCA
GCGGCAGCGGCAGCGGCACCGACTTCACCCTGACCATCAGGGGCCTGGAG
CCCGAGGACTTCGGCCTGTACTACTGCCAGCAGCACAACGAGTACCCCCC
CACCTTCGGCGCCGGCACCAAGCTGGAGCTGAAGGGAGGCGGAGGCTCTG
GCGGAGGCGGCTCTGGCGGAGGCGGCTCTCAGCTGCAGCTGGTGCAGAGC
GGCCCCGAGCTGAGGGAGCCCGGCGAGAGCGTGAAGATCAGCTGCAAGGC
CAGCGGCTACACCTTCACCGACTACATCGTGCACTGGGTGAAGCAGGCCC
CCGGCAAGGGCCTGAAGTGGATGGGCTGGATCAACACCTACACCGGCACC
CCCACCTACGCCGACGACTTCGAGGGCAGGTTCGTGTTCAGCCTGGAGGC
CAGCGCCAGCACCGCCAACCTGCAGATCAGCAACCTGAAGAACGAGGACA
CCGCCACCTACTTCTGCGCCAGGAGCTGGAGGAGGGGCATCAGGGGCATC
GGCTTCGACTACTGGGGCCAGGGCGTGATGGTGACCGTGAGCAGCttcgt
gcctgtgtttctgcctgccaagcccaccacaacccctgcccctagacctc
ctacacccgcccctacaatcgccagccagcctctgtctctgaggcccgag
gcttgtagacctgctgctggcggagccgtgcacaccagaggactggattt
cgcctgcgacatctacatctgggcccctctggccggcacatgtggcgtgc
tgctgctgagcctcgtgatcaccctgtactgcaaccaccggaacagaagc
aagcggagccggctgctgcacagcgactacatgaacatgacccccagacg
gcctggccccaccagaaagcactaccagccttacgcccctcccagagact
tcgccgcctaccggtccagagtgaagttcagcagaagcgccgacgcccct
gcctatcagcagggccagaaccagctgtacaacgagctgaacctgggcag
acgggaagagtacgatgtgctggacaaaagacgtggccgggaccctgaga
tggggggaaagccgagaaggaagaaccctcaggaaggcctgtacaatgaa
ctgcagaaagataagatggcggaggcctacagtgagattgggatgaaagg
cgagcgccggaggggcaaggggcacgatggcctttaccagggtctcagta
cagccaccaaggacacctacgacgcccttcacatgcaggccctgccccct
cgc

Preferably, therefore, the CAR construct is encoded by a nucleotide sequence substantially as set out in SEQ ID No: 34, or a fragment or variant thereof.

### “MPB2D5 CAR”

In yet another preferred embodiment, the anti-TCRVβ CAR construct is an anti-TCRVβ2 CAR (known as “MPB2D5 CAR”). Preferably, the anti-TCRVβ2 CAR comprises a 2×G₄S linker. In an embodiment, the anti-TCRVβ2 CAR (having a 2×G₄S linker) has an amino acid sequence referred to herein as SEQ ID No:35, as follows:

[SEQ ID No: 35]
MALPVTALLLPLALLLHAARPDIVLTQSPASLAVSLGQRATISCRASKSV
SILGTHLIHWYQQKPGQPPKLLIYAASNLESGVPARFSGSGSETVFTLNI
HPVEEEDAATYFCQQSIEDPWTFGGGTKLGIKGGGGSGGGGSEVQLQQSV
ADLVRPGASLKLSCTASGFNIKSAYMHWVIQRPDQGPECLGRIDPATGKT
KYAPKFQAKATITADTSSNTAYLQLSSLTSEDTAIYYCTRSLNWDYGLDY
WGQGTSVTVSSFVPVFLPAKPTTTPAPRPPTPAPTIASQPLSLRPEACRP
AAGGAVHTRGLDFACDIYIWAPLAGTCGVLLLSLVITLYCNHRNRSKRSR
LLHSDYMNMTPRRPGPTRKHYQPYAPPRDFAAYRSRVKFSRSADAPAYQQ
GQNQLYNELNLGRREEYDVLDKRRGRDPEMGGKPRRKNPQEGLYNELQKD
KMAEAYSEIGMKGERRRGKGHDGLYQGLSTATKDTYDALHMQALPPR

Preferably, therefore, the CAR construct comprises an amino acid sequence substantially as set out in SEQ ID No: 35, or a fragment or variant thereof.

In one embodiment, a nucleotide sequence encoding the anti-TCRVβ2 CAR (with 2×G₄S linker) is referred to herein as SEQ ID No: 36, as follows:

[SEQ ID No: 36]
atggctctgcctgtgacagctctgctgctgcctctggccctgctgctgca
tgccgccagacctGACATCGTGCTGACCCAGAGCCCCGCCAGCCTGGCCG
TGAGCCTGGGCCAGAGGGCCACCATCAGCTGCAGGGCCAGCAAGAGCGTG
AGCATCCTGGGCACCCACCTGATCCACTGGTACCAGCAGAAGCCCGGCCA
GCCCCCCAAGCTGCTGATCTACGCCGCtAGCAACCTGGAGAGCGGCGTGC
CCGCCAGGTTCAGCGGCAGCGGCAGCGAGACCGTGTTCACCCTGAACATC
CACCCCGTGGAGGAGGAGGACGCCGCCACCTACTTCTGCCAGCAGAGCAT
CGAGGACCCCTGGACCTTCGGCGGCGGCACCAAGCTGGGCATCAAGGGAG
GCGGAGGCTCTGGCGGAGGCGGCTCTGAGGTGCAGCTGCAGCAGAGCGTG
GCCGACCTGGTGAGGCCCGGCGCCAGCCTGAAGCTGAGCTGCACCGCCAG
CGGCTTCAACATCAAGAGCGCCTACATGCACTGGGTGATCCAGAGGCCCG
ACCAGGGCCCCGAGTGCCTGGGCAGGATCGACCCCGCCACCGGCAAGACC
AAGTACGCCCCCAAGTTCCAGGCCAAGGCCACCATCACCGCCGACACCAG
CAGCAACACCGCCTACCTGCAGCTGAGCAGCCTGACCAGCGAGGACACCG
CCATCTACTACTGCACCAGGAGCCTGAACTGGGACTACGGCCTGGACTAC
TGGGGCCAGGGCACCAGCGTGACCGTGAGCAGCttcgtgcctgtgtttct
gcctgccaagcccaccacaacccctgcccctagacctcctacacccgccc
ctacaatcgccagccagcctctgtctctgaggcccgaggcttgtagacct
gctgctggcggagccgtgcacaccagaggactggatttcgcctgcgacat
ctacatctgggcccctctggccggcacatgtggcgtgctgctgctgagcc
tcgtgatcaccctgtactgcaaccaccggaacagaagcaagcggagccgg
ctgctgcacagcgactacatgaacatgacccccagacggcctggccccac
cagaaagcactaccagccttacgcccctcccagagacttcgccgcctacc
ggtccagagtgaagttcagcagaagcgccgacgcccctgcctatcagcag
ggccagaaccagctgtacaacgagctgaacctgggcagacgggaagagta
cgatgtgctggacaaaagacgtggccgggaccctgagatggggggaaagc
cgagaaggaagaaccctcaggaaggcctgtacaatgaactgcagaaagat
aagatggcggaggcctacagtgagattgggatgaaaggcgagcgccggag
gggcaaggggcacgatggcctttaccagggtctcagtacagccaccaagg
acacctacgacgcccttcacatgcaggccctgccccctcgc

Preferably, therefore, the CAR construct is encoded by a nucleotide sequence substantially as set out in SEQ ID No: 36, or a fragment or variant thereof.

In another preferred embodiment, the anti-TCRVβ2 CAR construct (known as “MPB2D5 CAR”) comprises a 3×G₄S linker. In one embodiment, the anti-TCRVβ2 CAR (having a 3×G₄S linker) can have an amino acid sequence referred to herein as SEQ ID No: 37, as follows:

[SEQ ID No: 37]
MALPVTALLLPLALLLHAARPDIVLTQSPASLAVSLGQRATISCRASKSV
SILGTHLIHWYQQKPGQPPKLLIYAASNLESGVPARFSGSGSETVFTLNI
HPVEEEDAATYFCQQSIEDPWTFGGGTKLGIKGGGGSGGGGSGGGGSEVQ
LQQSVADLVRPGASLKLSCTASGENIKSAYMHWVIQRPDQGPECLGRIDP
ATGKTKYAPKFQAKATITADTSSNTAYLQLSSLTSEDTAIYYCTRSLNWD
YGLDYWGQGTSVTVSSFVPVELPAKPTTTPAPRPPTPAPTIASQPLSLRP
EACRPAAGGAVHTRGLDFACDIYIWAPLAGTCGVLLLSLVITLYCNHRNR
SKRSRLLHSDYMNMTPRRPGPTRKHYQPYAPPRDFAAYRSRVKFSRSADA
PAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALP
PR

Preferably, therefore, the CAR construct comprises an amino acid sequence substantially as set out in SEQ ID No: 37, or a fragment or variant thereof.

In one embodiment, a nucleotide sequence encoding the anti-TCRVβ2 CAR (with a 3×G₄S linker) is referred to herein as SEQ ID No: 38, as follows:

[SEQ ID No: 38]
atggctctgcctgtgacagctctgctgctgcctctggccctgctgctgca
tgccgccagacctGACATCGTGCTGACCCAGAGCCCCGCCAGCCTGGCCG
TGAGCCTGGGCCAGAGGGCCACCATCAGCTGCAGGGCCAGCAAGAGCGTG
AGCATCCTGGGCACCCACCTGATCCACTGGTACCAGCAGAAGCCCGGCCA
GCCCCCCAAGCTGCTGATCTACGCCGCtAGCAACCTGGAGAGCGGCGTGC
CCGCCAGGTTCAGCGGCAGCGGCAGCGAGACCGTGTTCACCCTGAACATC
CACCCCGTGGAGGAGGAGGACGCCGCCACCTACTTCTGCCAGCAGAGCAT
CGAGGACCCCTGGACCTTCGGCGGCGGCACCAAGCTGGGCATCAAGGGAG
GCGGAGGCTCTGGCGGAGGCGGCTCTGGCGGAGGCGGCTCTGAGGTGCAG
CTGCAGCAGAGCGTGGCCGACCTGGTGAGGCCCGGCGCCAGCCTGAAGCT
GAGCTGCACCGCCAGCGGCTTCAACATCAAGAGCGCCTACATGCACTGGG
TGATCCAGAGGCCCGACCAGGGCCCCGAGTGCCTGGGCAGGATCGACCCC
GCCACCGGCAAGACCAAGTACGCCCCCAAGTTCCAGGCCAAGGCCACCAT
CACCGCCGACACCAGCAGCAACACCGCCTACCTGCAGCTGAGCAGCCTGA
CCAGCGAGGACACCGCCATCTACTACTGCACCAGGAGCCTGAACTGGGAC
TACGGCCTGGACTACTGGGGCCAGGGCACCAGCGTGACCGTGAGCAGCtt
cgtgcctgtgtttctgcctgccaagcccaccacaacccctgcccctagac
ctcctacacccgcccctacaatcgccagccagcctctgtctctgaggccc
gaggcttgtagacctgctgctggcggagccgtgcacaccagaggactgga
tttcgcctgcgacatctacatctgggcccctctggccggcacatgtggcg
tgctgctgctgagcctcgtgatcaccctgtactgcaaccaccggaacaga
agcaagcggagccggctgctgcacagcgactacatgaacatgacccccag
acggcctggccccaccagaaagcactaccagccttacgcccctcccagag
acttcgccgcctaccggtccagagtgaagttcagcagaagcgccgacgcc
cctgcctatcagcagggccagaaccagctgtacaacgagctgaacctggg
cagacgggaagagtacgatgtgctggacaaaagacgtggccgggaccctg
agatggggggaaagccgagaaggaagaaccctcaggaaggcctgtacaat
gaactgcagaaagataagatggcggaggcctacagtgagattgggatgaa
aggcgagcgccggaggggcaaggggcacgatggcctttaccagggtctca
gtacagccaccaaggacacctacgacgcccttcacatgcaggccctgccc
cctcgc

Preferably, therefore, the CAR construct is encoded by a nucleotide sequence substantially as set out in SEQ ID No: 38, or a fragment or variant thereof.

### “FIN9 CAR”

In one preferred embodiment the anti-TCRVβ CAR construct is an anti-TCRVβ9 CAR (known as “FIN9 CAR”). Preferably, the anti-TCRVβ9 CAR comprises a 2×G₄S linker. In one embodiment, the anti-TCRVβ9 CAR construct has an amino acid sequence referred to herein as SEQ ID No:39, as follows:

[SEQ ID No: 39]
MALPVTALLLPLALLLHAARPETTVTQSPASLSVATGEKVTIRCISSTDI
DDDMNWYQQKSGEPPKLLISEGNTLRPGVPSRFSSSGYGTDFVFTIENML
SEDVADYYCLQSDNMPLTFGAGTKLELKGGGGSGGGGSEVQLQQSVAELV
RPGASVKLSCTASGFNIKNTFMHWVKQRPEQGLEWIGRIDPTNGYTKFAP
KFQGKATLTAVTSSNTVYLQLSSLTSEDTAIYYCAHDYDAPWFAYWGQGT
LVIVSAFVPVFLPAKPTTTPAPRPPTPAPTIASQPLSLRPEACRPAAGGA
VHTRGLDFACDIYIWAPLAGTCGVLLLSLVITLYCNHRNRSKRSRLLHSD
YMNMTPRRPGPTRKHYQPYAPPRDFAAYRSRVKFSRSADAPAYQQGQNQL
YNELNLGRREEYDVLDKRRGRDPEMGGKPRRKNPQEGLYNELQKDKMAEA
YSEIGMKGERRRGKGHDGLYQGLSTATKDTYDALHMQALPPR

Preferably, therefore, the CAR construct comprises an amino acid sequence substantially as set out in SEQ ID No:39, or a fragment or variant thereof.

In one embodiment, a nucleotide sequence encoding the anti-TCRVβ9 CAR (with 2×G₄S linker) is referred to herein as SEQ ID No: 40, as follows:

[SEQ ID No: 40]
atggctctgcctgtgacagctctgctgctgcctctggccctgctgctgca
tgccgccagacctGAGACCACCGTGACCCAGAGCCCCGCCAGCCTGAGCG
TGGCCACCGGCGAGAAGGTGACCATCAGGTGCATCAGCAGCACCGACATC
GACGACGACATGAACTGGTACCAGCAGAAGAGCGGCGAGCCCCCCAAGCT
GCTGATCAGCGAGGGCAACACCCTGAGGCCCGGCGTGCCCAGCAGGTTCA
GCAGCAGCGGCTACGGCACCGACTTCGTGTTCACCATCGAGAACATGCTG
AGCGAGGACGTGGCCGACTACTACTGCCTGCAGAGCGACAACATGCCCCT
GACCTTCGGCGCCGGCACCAAGCTGGAGCTGAAGGGAGGCGGAGGCTCTG
GCGGAGGCGGCTCTGAGGTGCAGCTGCAGCAGAGCGTGGCCGAGCTGGTG
AGGCCCGGCGCCAGCGTGAAGCTGAGCTGCACCGCCAGCGGCTTCAACAT
CAAGAACACCTTCATGCACTGGGTGAAGCAGAGGCCCGAGCAGGGCCTGG
AGTGGATCGGCAGGATCGACCCCACCAACGGCTACACCAAGTTCGCCCCC
AAGTTCCAGGGCAAGGCCACCCTGACCGCCGTGACCAGCAGCAACACCGT
GTACCTGCAGCTGAGCAGCCTGACCAGCGAGGACACCGCCATCTACTACT
GCGCCCACGACTACGACGCCCCCTGGTTCGCCTACTGGGGCCAGGGCACC
CTGGTGATCGTGAGCGCCttcgtgcctgtgtttctgcctgccaagcccac
cacaacccctgcccctagacctcctacacccgcccctacaatcgccagcc
agcctctgtctctgaggcccgaggcttgtagacctgctgctggcggagcc
gtgcacaccagaggactggatttcgcctgcgacatctacatctgggcccc
tctggccggcacatgtggcgtgctgctgctgagcctcgtgatcaccctgt
actgcaaccaccggaacagaagcaagcggagccggctgctgcacagcgac
tacatgaacatgacccccagacggcctggccccaccagaaagcactacca
gccttacgcccctcccagagacttcgccgcctaccggtccagagtgaagt
tcagcagaagcgccgacgcccctgcctatcagcagggccagaaccagctg
tacaacgagctgaacctgggcagacgggaagagtacgatgtgctggacaa
aagacgtggccgggaccctgagatggggggaaagccgagaaggaagaacc
ctcaggaaggcctgtacaatgaactgcagaaagataagatggcggaggcc
tacagtgagattgggatgaaaggcgagcgccggaggggcaaggggcacga
tggcctttaccagggtctcagtacagccaccaaggacacctacgacgccc
ttcacatgcaggccctgccccctcgc

Preferably, therefore, the CAR construct is encoded by a nucleotide sequence substantially as set out in SEQ ID No: 40, or a fragment or variant thereof.

In another preferred embodiment, the anti-TCRVβ9 CAR construct (known as “FIN9 CAR”) comprise a 3×G₄S linker. In one embodiment, the anti-TCRVβ9 CAR (having a 3×G₄S linker) can have an amino acid sequence referred to herein as SEQ ID No:41, as follows:

[SEQ ID No: 41]
MALPVTALLLPLALLLHAARPETTVTQSPASLSVATGEKVTIRCISSTDI
DDDMNWYQQKSGEPPKLLISEGNTLRPGVPSRFSSSGYGTDFVFTIENML
SEDVADYYCLQSDNMPLTFGAGTKLELKGGGGSGGGGSGGGGSEVQLQQS
VAELVRPGASVKLSCTASGFNIKNTFMHWVKQRPEQGLEWIGRIDPTNGY
TKFAPKFQGKATLTAVTSSNTVYLQLSSLTSEDTAIYYCAHDYDAPWFAY
WGQGTLVIVSAFVPVFLPAKPTTTPAPRPPTPAPTIASQPLSLRPEACRP
AAGGAVHTRGLDFACDIYIWAPLAGTCGVLLLSLVITLYCNHRNRSKRSR
LLHSDYMNMTPRRPGPTRKHYQPYAPPRDFAAYRSRVKFSRSADAPAYQQ
GQNQLYNELNLGRREEYDVLDKRRGRDPEMGGKPRRKNPQEGLYNELQKD
KMAEAYSEIGMKGERRRGKGHDGLYQGLSTATKDTYDALHMQALPPR

Preferably, therefore, the CAR construct comprises an amino acid sequence substantially as set out in SEQ ID No: 41, or a fragment or variant thereof.

In one embodiment, a nucleotide sequence encoding the anti-TCRVβ9 CAR (with 3×G₄S) is referred to herein as SEQ ID No: 42, as follows:

[SEQ ID No: 42]
atggctctgcctgtgacagctctgctgctgcctctggccctgctgctgca
tgccgccagacctGAGACCACCGTGACCCAGAGCCCCGCCAGCCTGAGCG
TGGCCACCGGCGAGAAGGTGACCATCAGGTGCATCAGCAGCACCGACATC
GACGACGACATGAACTGGTACCAGCAGAAGAGCGGCGAGCCCCCCAAGCT
GCTGATCAGCGAGGGCAACACCCTGAGGCCCGGCGTGCCCAGCAGGTTCA
GCAGCAGCGGCTACGGCACCGACTTCGTGTTCACCATCGAGAACATGCTG
AGCGAGGACGTGGCCGACTACTACTGCCTGCAGAGCGACAACATGCCCCT
GACCTTCGGCGCCGGCACCAAGCTGGAGCTGAAGGGAGGCGGAGGCTCTG
GCGGAGGCGGCTCTGGCGGAGGCGGCTCTGAGGTGCAGCTGCAGCAGAGC
GTGGCCGAGCTGGTGAGGCCCGGCGCCAGCGTGAAGCTGAGCTGCACCGC
CAGCGGCTTCAACATCAAGAACACCTTCATGCACTGGGTGAAGCAGAGGC
CCGAGCAGGGCCTGGAGTGGATCGGCAGGATCGACCCCACCAACGGCTAC
ACCAAGTTCGCCCCCAAGTTCCAGGGCAAGGCCACCCTGACCGCCGTGAC
CAGCAGCAACACCGTGTACCTGCAGCTGAGCAGCCTGACCAGCGAGGACA
CCGCCATCTACTACTGCGCCCACGACTACGACGCCCCCTGGTTCGCCTAC
TGGGGCCAGGGCACCCTGGTGATCGTGAGCGCCttcgtgcctgtgtttct
gcctgccaagcccaccacaacccctgcccctagacctcctacacccgccc
ctacaatcgccagccagcctctgtctctgaggcccgaggcttgtagacct
gctgctggcggagccgtgcacaccagaggactggatttcgcctgcgacat
ctacatctgggcccctctggccggcacatgtggcgtgctgctgctgagcc
tcgtgatcaccctgtactgcaaccaccggaacagaagcaagcggagccgg
ctgctgcacagcgactacatgaacatgacccccagacggcctggccccac
cagaaagcactaccagccttacgcccctcccagagacttcgccgcctacc
ggtccagagtgaagttcagcagaagcgccgacgcccctgcctatcagcag
ggccagaaccagctgtacaacgagctgaacctgggcagacgggaagagta
cgatgtgctggacaaaagacgtggccgggaccctgagatggggggaaagc
cgagaaggaagaaccctcaggaaggcctgtacaatgaactgcagaaagat
aagatggcggaggcctacagtgagattgggatgaaaggcgagcgccggag
gggcaaggggcacgatggcctttaccagggtctcagtacagccaccaagg
acacctacgacgcccttcacatgcaggccctgccccctcgc

Preferably, therefore, the construct is encoded by a nucleotide sequence substantially as set out in SEQ ID No: 42, or a fragment or variant thereof.

### “C21 CAR”

In yet another preferred embodiment, the anti-TCRVβ CAR construct is an anti-TCRVβ11 CAR (known as “C21 CAR”). Preferably, the anti-TCRVβ11 CAR comprises have a 2×G₄S linker. In one embodiment, the anti-TCRVβ11 CAR construct (having a 2×G₄S linker) can have an amino acid sequence referred to herein as SEQ ID No: 43, as follows:

[SEQ ID No: 43]
MALPVTALLLPLALLLHAARPDIKMTQSPSSMYASLGERVTITCKASQDI
NSYLSWFQQKAGKSPKTLIYRANRLVDGVPSRFSGSGSGQDYSLTISSLE
YEDMGIYYCLQYDEFPFTFGGGTRLEIKGGGGSGGGGSQVQLQQSGPEVV
RPGVSVKISCKGSGYRFTDSAMHWVKQSHAKSLEWIGVISSYNGNTNYNQ
KFKGKATMTVDKSSSTAYMELARMTSEDSAIYYCARSRDAMDYWGQGTSV
TVSSFVPVFLPAKPTTTPAPRPPTPAPTIASQPLSLRPEACRPAAGGAVH
TRGLDFACDIYIWAPLAGTCGVLLLSLVITLYCNHRNRSKRSRLLHSDYM
NMTPRRPGPTRKHYQPYAPPRDFAAYRSRVKFSRSADAPAYQQGQNQLYN
ELNLGRREEYDVLDKRRGRDPEMGGKPRRKNPQEGLYNELQKDKMAEAYS
EIGMKGERRRGKGHDGLYQGLSTATKDTYDALHMQALPPR

Preferably, therefore, the CAR construct comprises an amino acid sequence substantially as set out in SEQ ID No:43, or a fragment or variant thereof.

In one embodiment, a nucleotide sequence encoding the anti-TCRVβ11 CAR (with a 2×G₄S linker) is referred to herein as SEQ ID No: 52, as follows:

[SEQ ID No: 52]
ATGGCTCTGCCCGTCACAGCTCTGCTGCTGCCTCTGGCTCTGCTGCTGCA
CGCTGCTAGACCCGACATTAAGATGACCCAGTCCCCCTCCTCCATGTATG
CCAGCCTCGGCGAGAGAGTCACCATCACATGCAAGGCCAGCCAAGACATC
AACAGCTACCTCAGCTGGTTCCAGCAGAAAGCCGGCAAGAGCCCCAAGAC
ACTGATCTATAGGGCTAATAGACTGGTGGACGGCGTGCCTAGCAGATTTT
CCGGCAGCGGCAGCGGCCAAGACTATTCTCTGACCATCAGCTCTCTGGAG
TACGAGGACATGGGAATCTACTACTGTCTGCAGTACGACGAGTTCCCCTT
CACCTTCGGAGGCGGCACAAGACTGGAAATCAAAGGAGGAGGAGGATCCG
GAGGCGGAGGAAGCCAAGTGCAGCTCCAGCAGTCCGGACCCGAGGTGGTG
AGGCCCGGCGTGAGCGTGAAGATCAGCTGCAAGGGCAGCGGCTATAGGTT
CACCGACTCCGCCATGCACTGGGTGAAGCAGTCCCATGCCAAGAGCCTCG
AGTGGATCGGCGTGATCAGCAGCTACAACGGCAACACCAACTACAACCAG
AAGTTCAAGGGCAAGGCCACAATGACCGTGGACAAGAGCAGCTCCACCGC
CTACATGGAGCTGGCCAGAATGACCAGCGAGGATAGCGCCATCTACTACT
GTGCTAGGTCTAGAGACGCCATGGACTACTGGGGCCAAGGCACATCCGTG
ACCGTGAGCTCCTTTGTGCCCGTGTTTCTGCCCGCCAAGCCTACAACCAC
ACCCGCTCCTAGACCTCCCACCCCCGCCCCTACCATCGCTTCCCAGCCTC
TGTCTCTGAGACCCGAGGCTTGCAGACCCGCCGCTGGAGGCGCTGTGCAC
ACCAGAGGActggatttcgcctgcgacatctacatctgggcccctctggc
cggcacatgtggcgtgctgctgctgagcctcgtgatcaccctgtactgca
accaccggaacagaagcaagcggagccggctgctgcacagcgactacatg
aacatgacccccagacggcctggccccaccagaaagcactaccagcctta
cgcccctcccagagacttcgccgcctaccggtccagagtgaagttcagca
gaagcgccgacgcccctgcctatcagcagggccagaaccagctgtacaac
gagctgaacctgggcagacgggaagagtacgatgtgctggacaaaagacg
tggccgggaccctgagatggggggaaagccgagaaggaagaaccctcagg
aaggcctgtacaatgaactgcagaaagataagatggcggaggcctacagt
gagattgggatgaaaggcgagcgccggaggggcaaggggcacgatggcct
ttaccagggtctcagtacagccaccaaggacacctacgacgcccttcaca
tgcaggccctgccccctcgc

Preferably, therefore, the construct is encoded by a nucleotide sequence substantially as set out in SEQ ID No: 52, or a fragment or variant thereof.

In another preferred embodiment, the anti-TCRVβ11 CAR construct (known as “C21 CAR”) comprises a 3×G₄S linker. In one embodiment, the anti-TCRVβ11 CAR construct (having a 3×G₄S linker) can have an amino acid sequence referred to herein as SEQ ID No: 44, as follows:

[SEQ ID No: 44]
MALPVTALLLPLALLLHAARPDIKMTQSPSSMYASLGERVTITCKASQDI
NSYLSWFQQKAGKSPKTLIYRANRLVDGVPSRFSGSGSGQDYSLTISSLE
YEDMGIYYCLQYDEFPFTFGGGTRLEIKGGGGSGGGGSGGGGSQVQLQQS
GPEVVRPGVSVKISCKGSGYRFTDSAMHWVKQSHAKSLEWIGVISSYNGN
TNYNQKFKGKATMTVDKSSSTAYMELARMTSEDSAIYYCARSRDAMDYWG
QGTSVTVSSFVPVFLPAKPTTTPAPRPPTPAPTIASQPLSLRPEACRPAA
GGAVHTRGLDFACDIYIWAPLAGTCGVLLLSLVITLYCNHRNRSKRSRLL
HSDYMNMTPRRPGPTRKHYQPYAPPRDFAAYRSRVKFSRSADAPAYQQGQ
NQLYNELNLGRREEYDVLDKRRGRDPEMGGKPRRKNPQEGLYNELQKDKM
AEAYSEIGMKGERRRGKGHDGLYQGLSTATKDTYDALHMQALPPR

Preferably, therefore, the CAR construct an amino acid sequence substantially as set out in SEQ ID No: 44, or a fragment or variant thereof.

In one embodiment, a nucleotide sequence encoding the anti-TCRVβ11 CAR (with a 3×G₄S linker) is referred to herein as SEQ ID No: 45, as follows:

[SEQ ID No: 45]
ATGGCTCTGCCCGTCACAGCTCTGCTGCTGCCTCTGGCTCTGCTGCTGCA
CGCTGCTAGACCCGACATTAAGATGACCCAGTCCCCCTCCTCCATGTATG
CCAGCCTCGGCGAGAGAGTCACCATCACATGCAAGGCCAGCCAAGACATC
AACAGCTACCTCAGCTGGTTCCAGCAGAAAGCCGGCAAGAGCCCCAAGAC
ACTGATCTATAGGGCTAATAGACTGGTGGACGGCGTGCCTAGCAGATTTT
CCGGCAGCGGCAGCGGCCAAGACTATTCTCTGACCATCAGCTCTCTGGAG
TACGAGGACATGGGAATCTACTACTGTCTGCAGTACGACGAGTTCCCCTT
CACCTTCGGAGGCGGCACAAGACTGGAAATCAAAGGAGGAGGAGGATCCG
GAGGCGGAGGAAGCGGAGGCGGAGGAAGCCAAGTGCAGCTCCAGCAGTCC
GGACCCGAGGTGGTGAGGCCCGGCGTGAGCGTGAAGATCAGCTGCAAGGG
CAGCGGCTATAGGTTCACCGACTCCGCCATGCACTGGGTGAAGCAGTCCC
ATGCCAAGAGCCTCGAGTGGATCGGCGTGATCAGCAGCTACAACGGCAAC
ACCAACTACAACCAGAAGTTCAAGGGCAAGGCCACAATGACCGTGGACAA
GAGCAGCTCCACCGCCTACATGGAGCTGGCCAGAATGACCAGCGAGGATA
GCGCCATCTACTACTGTGCTAGGTCTAGAGACGCCATGGACTACTGGGGC
CAAGGCACATCCGTGACCGTGAGCTCCTTTGTGCCCGTGTTTCTGCCCGC
CAAGCCTACAACCACACCCGCTCCTAGACCTCCCACCCCCGCCCCTACCA
TCGCTTCCCAGCCTCTGTCTCTGAGACCCGAGGCTTGCAGACCCGCCGCT
GGAGGCGCTGTGCACACCAGAGGActggatttcgcctgcgacatctacat
ctgggcccctctggccggcacatgtggcgtgctgctgctgagcctcgtga
tcaccctgtactgcaaccaccggaacagaagcaagcggagccggctgctg
cacagcgactacatgaacatgacccccagacggcctggccccaccagaaa
gcactaccagccttacgcccctcccagagacttcgccgcctaccggtcca
gagtgaagttcagcagaagcgccgacgcccctgcctatcagcagggccag
aaccagctgtacaacgagctgaacctgggcagacgggaagagtacgatgt
gctggacaaaagacgtggccgggaccctgagatggggggaaagccgagaa
ggaagaaccctcaggaaggcctgtacaatgaactgcagaaagataagatg
gcggaggcctacagtgagattgggatgaaaggcgagcgccggaggggcaa
ggggcacgatggcctttaccagggtctcagtacagccaccaaggacacct
acgacgcccttcacatgcaggccctgccccctcgc

Preferably, therefore, the CAR construct is encoded by a nucleotide sequence substantially as set out in SEQ ID No: 45, or a fragment or variant thereof.

In a second aspect, there is provided a nucleic acid encoding the CAR construct of the first aspect.

Preferably, the nucleic acid comprises a promoter operably linked to the sequence encoding the CAR construct. The promoter is preferably disposed 5′ of the sequence encoding the signalling peptide. The promoter drives expression of the CAR construct in a host cell.

The promoter may be any suitable promoter, including a constitutive promoter, an activatable promoter, an inducible promoter, or a tissue-specific promoter. Constitutive promoters allow heterologous genes (also referred to as transgenes) to be expressed constitutively in the host cells. Exemplary constitutive promoters contemplated herein include, but are not limited to, Cytomegalovirus (CMV) promoters, human elongation factors-1 alpha (hEFla), ubiquitin C promoter (UbiC), phosphoglycerokinase promoter (PGK), simian virus 40 early promoter (SV40), and chicken β-Actin promoter coupled with CMV early enhancer (CAGG). Inducible promoters belong to the category of regulated promoters. The inducible promoter can be induced by one or more conditions, such as a physical condition, microenvironment of the engineered immune effector cell, or the physiological state of the engineered immune effector cell, an inducer (i.e., an inducing agent), or a combination thereof. In some embodiments, the inducing condition does not induce the expression of endogenous genes in the engineered mammalian cell, and/or in the subject that receives the pharmaceutical composition. In some embodiments, the inducing condition is selected from the group consisting of: inducer, irradiation (such as ionizing radiation, light), temperature (such as heat), redox state, tumor environment, and the activation state of the engineered mammalian cell.

In one embodiment, the promoter may be the PGK promoter (EMBL NO: A19297.1). In an embodiment, the PGK promoter is referred to herein as SEQ ID No: 48, as follows:

[SEQ ID No: 48]
GGGTAGGGGAGGCGCTTTTCCCAAGGCAGTCTGGAGCATGCGCTTTAGCA
GCCCCGCTGGGCACTTGGCGCTACACAAGTGGCCTCTGGCCTCGCACACA
TTCCACATCCACCGGTAGGCGCCAACCGGCTCCGTTCTTTGGTGGCCCCT
TCGCGCCACCTTCTACTCCTCCCCTAGTCAGGAAGTTCCCCCCCGCCCCG
CAGCTCGCGTCGTGCAGGACGTGACAAATGGAAGTAGCACGTCTCACTAG
TCTCGTGCAGATGGACAGCACCGCTGAGCAATGGAAGCGGGTAGGCCTTT
GGGGCAGCGGCCAATAGCAGCTTTGCTCCTTCGCTTTCTGGGCTCAGAGG
CTGGGAAGGGGTGGGTCCGGGGGCGGGCTCAGGGGGGGGCTCAGGGGCGG
GGGGGGCGCCCGAAGGTCCTCCGGAGGCCCGGCATTCTGCACGCTTCAAA
AGCGCACGTCTGCCGCGCTGTTCTCCTCTTCCTCATTCTCCGGGCCTTTC
G

Preferably, therefore, the promoter may comprise a nucleotide sequence substantially as set out in SEQ ID No: 48, or a fragment or variant thereof.

In a preferred embodiment, the nucleic acid of the second aspect is selected from any of the nucleic acid sequences described herein.

In a third aspect, there is provided an expression vector or a plasmid encoding the CAR construct of the first aspect, or comprising the nucleic acid of the second aspect.

Preferably, the vector is recombinant. Preferably, the vector is a viral vector, more preferably a retroviral vector, even more preferably a lentivirus. Maps showing the vector's plasmid structure of exemplary anti-TCRVβ CAR constructs tested in the present invention are shown in FIGS. 4-7.

Preferably, the vector comprises left (i.e. N-term) and/or right (i.e. C-term) Long Terminal Repeat sequences (LTRs). Preferably, each LTR is disposed at the N-term and/or C-term of the construct.

In a preferred embodiment, the vector comprises a 5′/N-term LTR-a promoter-a variable light chain of a scFv domain of an anti-TCRVβ antibody-a linker-a variable heavy chain of a scFv domain of an anti-TCRVβ antibody-a hinge-a co-stimulatory domain comprising CD28-a stimulatory protein CD3ζ-a 3′/C-term LTR.

Preferably, the hinge comprises a CD8α hinge, and optionally, a transmembrane and/or cytoplasmic domain.

In a fourth aspect, there is provided an effector cell expressing the CAR construct of the first aspect, or comprising the nucleic acid of the second aspect or the vector according to the third aspect.

The vector may be a lenti-virus vector. The vector may be a retroviral vector.

Preferably, the effector cell is a normal or conventional αβT-cell, or an innate lymphocyte, such as an invariant natural killer T (INKT) cell, γδT cell or NK cell.

The skilled person is aware that conventional αβT-cells are one of two primary types of lymphocytes (B cells being the second type) which determine the specificity of an immune response to antigens (foreign substances) in the body. T cells coordinate multiple aspects of adaptive immunity throughout life, including responses to pathogens, allergens, and tumours through the production of cytokines and effector molecules. In humans, T cells control multiple insults simultaneously throughout the body and maintain immune homeostasis over decades.

Thus, in one embodiment, the effector cell of the invention is a conventional T-cell.

A “conventional T-cell” may be defined as a T lymphocyte that expresses an αβ T cell receptor (TCR), as well as a co-receptor CD4 or CD8, and is present in the peripheral blood, lymph nodes, and tissues.

In another preferred embodiment, however, the effector cell is an iNKT cell. iNKT cells are a subset of immunoregulatory and effector T cells representing less than 0.1% of the total T cell numbers in human.

There are several structural and functional differences between iNKT cells and conventional T cells. In particular, iNKT cells express an invariant Va24Ja18 chain which is almost always paired with the same TCRVβ11 diverse chain. Furthermore, iNKT cells are also restricted by the non-polymorphic HLA class I-like molecule CD1d presenting endogenous or exogenous, glyco- or phospho-lipid ligands to iTCR. In contrast to conventional T-cells, which are restricted by highly polymorphic MHC presenting peptides, iNKT cells require the expression of CD1d on thymocytes in order to be selected and activated. Conversely, conventional T-cells require the expression of MHC molecules on epithelial thymic epithelial cells for their selection and activation.

INKT cells provide effective immune responses against infectious agents, tumour, allo- and auto-reactivity and atheromatosis. Several pre-clinical studies have demonstrated the ability of adoptively transferred donor iNKT cells to prevent or even abrogate established experimental acute graft-versus-host disease (aGVHD), an alloreactive phenomenon that occurs in the context of allogeneic haemopoietic stem cell transplantation. aGVHD is driven primarily by donor alloreactive T cells activated in response to major or minor histocompatibility antigen disparities between donor and recipient. In line with the pre-clinical evidence, several clinical observational studies have demonstrated that a higher dose or frequency of donor iNKT cells transferred to the recipient with the peripheral blood stem cell graft impart significant protection from aGVHD without compromising the graft-versus-tumour effect.

In humans, iNKT cells are quantitatively and qualitatively altered in different types of tumours, including blood cancers such as multiple myeloma, while tumour bed infiltration by iNKT cells appears to confer favourable prognosis in colorectal cancer.

Much of the anti-tumour effect of iNKT cells depends on their ability to be cytolytic directly, through perforin/granzymes and other cell death pathways against tumours that express CD1d, or indirectly, through their secretion of copious amounts of (interferon-gamma) IFNγ, and/or secondary activation of NK cells or conventional T cell-dependent anti-tumour responses.

It will be appreciated that the effector cell of the fourth aspect is produced by transducing a T-cell or an iNKT cell with the nucleic acid or the vector encoding the CAR construct.

Thus, in a fifth aspect, there is provided a method of producing an effector cell expressing an anti-TCRVβ CAR, the method comprising transducing an effector cell with the nucleic acid according to the second aspect or the vector according to the third aspect, such that the effector cell expresses an anti-TCRVβ CAR.

Preferably, the effector cell is a normal or conventional αβBT-cell, or an innate lymphocyte, such as an invariant natural killer T (INKT) cell, γδT cell or NK cell.

Preferably, the method comprises an initial step of isolating the effector cell from peripheral blood cells (PBCs). Preferably, the effector cell is activated with one or both of CD3 and CD28 antibodies. Preferably, the effector cell is activated with an interleukin. The interleukin may be IL-15. In a preferred embodiment, the effector cell is activated with CD3 and CD28 antibodies and IL-15.

In a preferred embodiment, the nucleic acid and/or vector encoding the anti-TCRVβ CAR may comprise any of nucleic acids or vectors described herein.

In a sixth aspect, there is provided a pharmaceutical composition comprising a therapeutically effective amount of the CAR construct according to the first aspect, the nucleic acid of the second aspect, the vector of the third aspect, or the effector cell according to the fourth aspect and a pharmaceutically acceptable excipient.

Preferably, the effector cell is a normal (i.e., conventional αβBT-cell), or an innate lymphocyte, such as an invariant natural killer T (INKT) cell, γδT cell or NK cell.

Preferably, the pharmaceutical composition comprises a plurality of the effector cell of the invention, preferably a T cell, or iNKT cell. For example, the composition may comprise at least 100, 1000, or 10,000 effector cells. Preferably, the composition comprises at least 100,000, or at least 1,000,000 or at least 10,000,000 effector cells.

In a seventh aspect, there is provided the CAR construct according to the first aspect, the nucleic acid of the second aspect, the vector of the third aspect, the effector cell according to the fourth aspect, or the pharmaceutical composition of the six aspect, for use in therapy or diagnosis.

In an eighth aspect, there is provided the CAR construct according to the first aspect, the nucleic acid of the second aspect, the vector of the third aspect, the effector cell according to the fourth aspect, or the pharmaceutical composition of the six aspect, for use in (i) immunotherapy; (ii) for treating, preventing or ameliorating cancer; (iii) for treating, preventing or ameliorating an autoimmune disease; or (iv) for treating, preventing or ameliorating any disease characterised by the presence of pathogenic T cells.

In a ninth aspect, the invention provides a method of: (i) treating, preventing or ameliorating a disease in a subject with immunotherapy; (ii) treating, preventing or ameliorating cancer; (iii) for treating, preventing or ameliorating an autoimmune disease in a subject; or (iv) for treating, preventing or ameliorating any disease characterised by the presence of pathogenic T cells, the method comprising administering, or having administered, to a patient in need of such treatment, a therapeutically effective amount of the CAR construct according to the first aspect, the nucleic acid of the second aspect, the vector of the third aspect, the effector cell according to the fourth aspect, or the pharmaceutical composition of the six aspect.

In one embodiment of the eighth or ninth aspect of the invention, the CAR construct, effector cell or pharmaceutical composition is for use in treating, preventing or ameliorating cancer. Preferably, the cancer is a T-cell malignancy, which may be a solid tumour or a liquid tumour.

As per WHO 2022 classification of hematolymphoid neoplasms, the T-cell neoplasms may be precursor T-cell neoplasms, such as T-lymphoblastic leukaemia/lymphoma or mature T-cell neoplasms, or Peripheral T cell lymphoma (PTCL). Collectively, with current treatments, the five year survival is 35-40%. As per WHO 2022 classification, mature T-cell neoplasms comprise a diverse group of uncommon and aggressive diseases in which the patient's T cells become cancerous. They are divided into three categories, i.e. nodal, extranodal and leukaemic, each of which are encompassed by the invention.

Mature T-cell neoplasm may be a TCL subtype selected from a group consisting of: Mature T-cell leukaemias; Primary cutaneous T-cell lymphoid proliferations and lymphomas; Intestinal T-cell lymphoid proliferations and lymphomas; Hepatosplenic T-cell lymphoma; Anaplastic large cell lymphoma; Nodal T-follicular helper (TFH) cell lymphoma; Peripheral T-cell lymphoma, NOS; EBV-positive NK-cell and T-cell lymphomas; EBV-positive T-cell lymphoid proliferations and lymphomas of childhood and all specific TCL entities within each of these subtypes as per WHO classification.

Adult T-Cell Leukaemia/Lymphoma (ATL) is more commonly found in Japan and the Caribbean than in the United States, and is associated with the human T-cell leukaemia virus type-1 (HTLV-1), rare and aggressive disease that starts in the liver or spleen and usually affects young adults in their 20s and 30s. Treatment for patients with hepatosplenic T-cell lymphoma includes anthracycline-based chemotherapy and, in some cases, stem cell transplantation.

Subcutaneous Panniculitis-Like Lymphoma (SPTCL) is the rarest and least well-defined of the T-cell lymphomas. This lymphoma occurs primarily in the subcutaneous fat tissue, where it causes nodules to form. Symptoms include fever, chills, weight loss and oral mucosal ulcers. SPTCL may be either rapidly aggressive or indolent (slow growing). Treatment includes combination anthracycline-based chemotherapy or localized radiation. Precursor T-Cell Acute Lymphoblastic Lymphoma or Leukaemia may be diagnosed as leukaemia or lymphoma or both. This cancer is found in both children and adults and is most commonly diagnosed in adolescent and adult males. Treatment for newly diagnosed patients with precursor T-cell acute lymphoblastic lymphoma or leukemia is aggressive chemotherapy and radiation. Nelarabine (Arranon®) is approved for the treatment of relapsed or refractory precursor T-cell acute lymphoblastic lymphoma or leukemia in adults and children.

Angioimmunoblastic T-cell lymphoma (AITL) exemplifies a neoplasm characterized by intense inflammatory and immune reactions, as evidenced by its clinical, pathologic, cellular, and biologic properties. Because tumour cells phenotypically resemble T follicular helper (Tfh) cells, they are considered to function similarly to some extent to nonneoplastic Tfh cells seen in reactive follicular hyperplasia. However, follicles are not hyperplastic but are rather depleted or destroyed in vast majority of AITL cases. AITL was recently reported to account for 36.1% of PTCL.

Cutaneous T-cell lymphoma (CTCL) constitute about 70-75% of the primary cutaneous lymphomas. The CTCL may be a CTCL subtype selected from a group consisting of: Mycosis fungoides (MF); Sezary syndrome (SS); and CD4+ small medium pleomorphic T-cell lymphoproliferative disorder.

Mycosis fungoides (MF) is the most common subtype. Sezary syndrome (SS) is a more aggressive type of CTCL. Patients with SS have erythroderma (i.e. rash affecting >80% body surface area [BSA]), lymphadenopathy, and high numbers of circulating neoplastic CD4+ T cells in the peripheral blood.

In other embodiments, the CAR construct, effector cell or pharmaceutical composition may be used in treating any disease caused by pathogenic T cells.

In other embodiments, the CAR construct, effector cell or pharmaceutical composition may be used in treating, preventing or ameliorating an autoimmune disease. The autoimmune disease may be caused by pathogenic autoreactive T cells, or selected from a group consisting of: systemic lupus erythematosus, rheumatoid arthritis, and myasthenia gravis.

It will be appreciated that the CAR construct, nucleic acid, vector, effector cell or pharmaceutical composition according to the invention (collectively referred to herein as “agents”) may be used in a monotherapy (e.g. the use of the CAR construct, nucleic acid, vector, effector cell or pharmaceutical composition alone), for therapy, preferably use in (i) immunotherapy; (ii) for treating, preventing or ameliorating cancer; (iii) for treating, preventing or ameliorating an autoimmune disease; or (iv) for treating, preventing or ameliorating any disease characterised by the presence of pathogenic T cells. Alternatively, CAR construct, effector cell or pharmaceutical composition according to the invention may be used as an adjunct to, or in combination with, known immunotherapies or for treating disease caused by pathogenic T cells, as well as cancers or autoimmune disease.

The agents according to the invention may be combined in compositions having a number of different forms depending, in particular, on the manner in which the composition is to be used. Thus, for example, the composition may be in the form of a liquid, preferably delivered intravenously to a person in need of treatment. It will be appreciated that the vehicle of medicaments according to the invention should be one which is well-tolerated by the subject to whom it is given.

In a preferred embodiment, agents and medicaments according to the invention may be administered to a subject by injection into the blood stream or directly into a site requiring treatment. Injections may be intravenous (bolus or infusion) or subcutaneous (bolus or infusion), or intradermal (bolus or infusion), or directly into tumours.

It will be appreciated that the amount of the CAR construct or the vector or effector cell (i.e. agent) that is required is determined by its biological activity and bioavailability, which in turn depends on the mode of administration, the physiochemical properties of the agent, and whether it is being used as a monotherapy or in a combined therapy. The frequency of administration will also be influenced by the in vivo persistence of the agent within the subject being treated. Optimal dosages to be administered may be determined by those skilled in the art, and will vary with the particular agent in use, the strength of the pharmaceutical composition, the mode of administration, and the advancement of the disease being treated, for example cancer, T-cell malignancies, or autoimmune disease. Additional factors depending on the particular subject being treated will result in a need to adjust dosages, including subject age, weight, gender, diet, and time of administration.

The invention also provides in a tenth aspect, a process for making the pharmaceutical composition according to the sixth aspect, the process comprising combining a therapeutically effective amount of the CAR construct according to the first aspect, the nucleic acid of the second aspect, the vector of the third aspect, or the effector cell according to the fourth aspect, and a pharmaceutically acceptable vehicle.

A “subject” may be a vertebrate, mammal, or domestic animal. Most preferably, the subject is a human being.

A “therapeutically effective amount” of the CAR construct, nucleic acid, vector, effector cell of composition is any amount which, when administered to a subject, is the amount of agent that is needed to treat the disease being treated, for example cancer, or produce the desired effect.

For example, the therapeutically effective amount of the effector cell used may be at least 100, 1000, or 10,000 effector cells. Preferably, at least 100,000, or at least 1,000,000 or at least 10,000,000 effector cells is used.

A “pharmaceutically acceptable vehicle” as referred to herein, is any known compound or combination of known compounds that are known to those skilled in the art to be useful in formulating pharmaceutical compositions. Preferably, for a successful CAR effective therapy, the composition comprising the CAR-effector cell is prepared and then delivered as a cell suspension, most preferably intravenously.

The pharmaceutical vehicle may be a liquid, and the pharmaceutical composition is in the form of a solution. Liquid vehicles are used in preparing solutions, suspensions, emulsions, syrups, elixirs and pressurized compositions. The active agent according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, intramuscular, intrathecal, epidural, intraperitoneal, intravenous and particularly subcutaneous injection. The agent may be prepared as a sterile solid composition that may be dissolved or suspended at the time of administration using sterile water, saline, or other appropriate sterile injectable medium.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with the sequences identified herein, and so on.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on: —(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula: —Sequence Identity=(N/T)*100.

Alternative methods for identifying similar sequences will be known to those skilled in the art. For example, a substantially similar nucleotide sequence will be encoded by a sequence which hybridizes to DNA sequences or their complements under stringent conditions. By stringent conditions, the inventors mean the nucleotide hybridises to filter-bound DNA or RNA in 3× sodium chloride/sodium citrate (SSC) at approximately 45 °C followed by at least one wash in 0.2×SSC/0.1% SDS at approximately 20-65 °C.

Alternatively, a substantially similar polypeptide may differ by at least 1, but less than 5, 10, 20, 50 or 100 amino acids from the sequences shown herein.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent (synonymous) change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example, small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstracts and drawings), and/or all of the steps of any method or process so disclosed, may be combined with any of the above aspects in any combination, except combinations where at least some features and/or steps are mutually exclusive.

For a better understanding of the invention, and to show how embodiments of the same may be carried into effect, reference will now be made, by way of example, to the accompanying Figures, in which: —

FIG. 1 shows a schematic map of one embodiment of a coding sequence of an expression vector for an anti-TCRVβ CAR lentiviral CAR of the invention. The sequence includes a human CD8α signal peptide, an antigen binding domain comprising a VL, flexible linker and VH, a CD8α hinge domain that includes a transmembrane domain and cytoplasmic domain, and an intracellular signalling domain comprising a CD28 co-stimulatory domain and CD3ζ stimulatory domain.

FIG. 2a shows the membrane topology of a mature CAR of the invention. The CD8α signal peptide, VL, linker, and VH domains form the extracellular ectodomain, and the CD8α hinge domain comprises an ectodomain part, a plasma membrane (P.M.) and a cytoplasmic fragment. The CD28 co-stimulatory domain and CD3ζ stimulatory domain form the endodomain inside the cell; FIG. 2b represents one embodiment of a CAR of the invention referred to as MPB2D5 CAR GS3 CD8283Z (TCRVB 2 CAR).

FIG. 3 provides structural details of the design of the coding sequence of each of various embodiments of exemplary anti-TCRVβ CAR of the invention. From top to bottom, the Figure shows CARs specific to TCRVβ1 CAR (i.e. anti-TCRVβ1 CAR), TCRVβ2 CAR (i.e. anti-TCRVβ2 CAR), TCRVβ9 CAR (i.e. anti-TCRVβ9 CAR), and TCRVβ11 CAR (i.e. anti-TCRVβ11 CAR). For each embodiment, a construct with 2×G₄S linker and a construct with a 3×G₄S linker, respectively, are illustrated. The position of each of the components of the construct (5′ to 3′) follows the order disclosed in the schematic map of FIG. 1.

FIG. 4 shows plasmid maps of the anti-TCRVβ1 CAR (called BL37.2 CAR) construct shown in FIG. 3. The top map is for a first embodiment of an anti-TCRVβ1 CAR comprising a 2×G₄S linker and the lower map is for a second embodiment of an anti-TCRVβ1 CAR comprising a 3×G₄S linker.

FIG. 5 shows plasmid maps of the anti-TCRVβ2 CAR (called MPB2D5 CAR) shown in FIG. 3. The top map is for a first embodiment of an anti-TCRVβ2 CAR comprising a 2×G₄S linker and the lower map is for a second embodiment of an anti-TCRVβ2 CAR comprising a 3×G₄S linker.

FIG. 6 shows the plasmid maps of the anti-TCRVβ9 CAR (called FIN9 CAR) shown in FIG. 3. The top map is for a first embodiment of an anti-TCRVβ9 CAR comprising a 2×G₄S linker and the lower map is for a second embodiment of an anti-TCRVβ9 CAR comprising a 3×G₄S linker.

FIG. 7 shows the plasmid maps of an anti-TCRVβ11 CAR (called C21 CAR) shown in FIG. 3. The top map is for a first embodiment of an anti-TCRVβ11 CAR comprising a 2×G₄S linker and the lower map is for a second embodiment of an anti-TCRVβ11 CAR comprising a 3×G₄S linker.

FIG. 8 shows the specificity, cytokine production and cytotoxic activity of anti-TCRVβ CAR-T cells of the invention. Peripheral blood mononuclear cells (PBMC) from a normal donor were cultured alone or transduced with lentivirus which encoded the anti-Vβ1, Vβ2 and Vβ9 CAR constructs. Five days post transduction, transduction efficiency was evaluated by protein L staining (see FIGS. 8A and B), and the frequency of CD3+ cells expressing each of the 24 TCRVβ subunits was quantified (see FIG. 8C). Values shown are the mean percentage of cells expressing each subunit normalised to the frequency of cells expressing that subunit in the non-transduced PBMC control from two (n=2) individuals. Killing of in-vitro expanded autologous primary T cell lines by anti-TCR CAR-T cells and anti-CD19 CAR-T (see FIG. 8D) and killing of non-transduced (UT) and TCR-GFP-transduced (Vβ+) JRT3-T3.5 cells by anti-Vβ CAR-T cells (see FIG. 8E). Target cells were stained with CFSE and co-cultured with effectors at a range of effector to target (E:T) ratios in duplicate. After 24 h, the cells were harvested and stained with 7aad. The frequency of dead targets in each culture condition was assayed by flow cytometry. Results are representative of two or more independent experiments. CD107a mobilisation, IFN-γ and TNF-α production by CD3+ anti-Vβ2 and anti-CD19 CAR-T when cultured alone or in the presence of untransduced JRT3-T3.5 cells (JRT), JRT3-T3.5 cells transduced with Vβ2+ TCR-GFP (TCR GFP), or primary cells expressing Vβ2 (1YT) (see FIG. 8F). Results are from a single experiment and are representative of two independent experiments.

FIG. 9 shows the cytokine production and cytotoxic activity of anti-TCRVβ CAR-INKT cells of the invention. Killing of in-vitro expanded autologous primary T cell lines by anti-TCR CAR-iNKT cells and anti-CD19 CAR-INKT (see FIG. 9A). Killing of untransduced (UT) and TCR-GFP-transduced (TCR-GFP) JRT-3 T 3.5 cells by anti-VB CAR-iNKT cells (see FIG. 9B). Target cells were stained with CFSE co-cultured with effectors at a range of effector to target (E:T) ratios in duplicate. After 24 h cells were harvested and stained with 7aad. The frequency of dead targets in each culture condition was assayed by flow cytometry. Results are representative of two or more independent experiments. CD107a mobilisation, IFN-γ and TNF-α production by CD3+ anti-Vβ2 and anti-CD19 CAR-T when cultured alone or in the presence of untransduced JRT3-T3.5 cells (JRT), JRT3-T3.5 cells transduced with Vβ2+ TCR-GFP (Vβ+), or primary cells expressing Vβ2 (1YT) (see FIG. 9C). Results are from a single experiment and are representative of 2 independent experiments. Enhancement of killing of α-gal loaded untransduced and TCRVβ1 or -Vβ2-GFP-transduced JRT3-T3.5 cells by anti-Vβ1 (see FIG. 9D) or Vβ2 CAR-iNKT but not anti Vβ1 or Vβ2 CAR-T cells (see FIG. 9E). Where indicated, target cells were incubated with 200 ng/ml α-gal before co-culture with effectors. Results shown are representative of two repeat experiments. In vivo protocol used is represented in FIG. 9F. 5×10⁶ TCRVβ+ JRT cells were suspended in matrigel and injected subcutaneously. On day 18, 1×10⁶ effector cells were injected intravenously into the tail vein. The challenged groups consisted of untreated (n=5), Vβ2 CAR-iNKT (n=7) or CD19 CAR-iNKT (n=5). Tumour volume for each group was measured periodically using calipers (see FIG. 9G). Tumours were excised and weighed at the end of the experiment (see FIG. 9H).

FIG. 10 shows the killing of ex-vivo Adult T cell Leukaemia/Lymphoma (ATL) cells by anti-Vβ CAR-INKT cells of the invention. Patient ATL01 had a malignant clone which expressed TCRVβ1, and ATL02 had a malignant clone which expressed TCRVβ2 (see FIG. 10A). Killing ex vivo of CD4+CCR4+CD26-ATL cells expressing TCRVβ1 (see FIG. 10B) and Vβ2 (see FIG. 10C) by anti-Vβ CAR-iNKT. Cryopreserved PBMC from two ATL patients and normal donor (ND) were stained with cell-trace violet and co-incubated with anti-Vβ1-Vβ2 or -CD19 CAR-iNKT at the indicated effector:target (PBMC) ratios in triplicate. After 24 h co-culture, cells were stained with a viability stain, anti-CD4, CCR4, CD26, TCRVβ1 or 2 and TCRαβ, fixed, and the frequency of dead CD4+CCR4+CD26− T cells and CCR4−CD26+/− cells (‘rest’ of CD4+ T cells) was determined by flow cytometry. *There were an insufficient number of cells to determine viability on CD4+ expressing other TCRVβ subunits in patient 2.

FIG. 11 shows the effect of CAR-iNKT cells on the frequency of antiviral CTL and HTLV-1 expressing CD4+ T cells. Frequency of M158-66 and Tax11-19 HLA-A*0201 pentamer+ CD3+ T cells after co-culture with CAR-iNKT (see FIGS. 11A and 11B). CD4-depleted PBMC from three HLA-A*0201+ HTLV-1 carriers were stained with CellTraceViolet and were cultured alone or with an 1:1 ratio of anti-Vβ1 -Vβ2 or -CD19 CAR-INKT. After 16-18 h co-culture, cells were stained with a viability stain, and anti-CD3, -CD8 antibodies and M₁₅₈₋₆₆ or Tax₁₁₋₁₉ pentamers. Cells were analysed by flow cytometry and the frequency of live Pentamer+CellTraceViolet+CD3+ cells was determined. The frequency of HTLV-1 Tax expressing CD4+ cells (see FIGS. 11C and 11D) after co-culture with CAR-iNKT is shown in FIGS. 11C and 11D. CellTraceViolet stained, positively selected CD4+ cells from the same donors were cultured alone or in the presence of Vβ2- or CD19-CAR iNKT. After 16-18h, cells were stained with a viability stain, anti-CD3, -CD4, -CD8 and -TCRVβ2. Cells were subsequently fixed, and stained intracellularly with anti-Tax antibody. Cells were analysed by flow cytometry and the frequency of live TCRVβ2+CD3+ cells and live Tax+CD4+ cells was determined.

FIG. 12 shows the raw data from FIG. 8c: i.e. the absolute frequency of CD3+ T cells expressing each TCRVβ subunit expressed as a percentage of total CD3+ T cells

FIG. 13 shows target cell lines for the CAR T cells of the invention. Primary T cells expressing Vβ subunits of interest were enriched using anti-TCRVβ-subunit antibodies and magnetic beads (see FIG. 13A). JRT3-T3.5 cells were transduced with lentiviral expression vectors encoding TCRβ chains and GFP (see FIG. 13B). Cells were sorted to enrich cells which expressed CD3 on the cell surface. One week post sorting, cells were stained with antibodies specific for CD3 and the relevant TCRVβ subunit and analysed by flow cytometry.

FIG. 14 shows the role played by iNKT iTCR cells of the invention in the killing of T cells. CD1d expression by in-vitro expanded primary T cells and JRT3-T3.5 cells (JRT) (see FIG. 14A). Cells were stained with a viability stain, anti-CD3 and anti-CD1d and were analysed by flow cytometry. CD3 and CD1d expression by live cells is shown. Mouse weight over the course of the in vivo experiment described in FIGS. 9F-9H. (see FIG. 14B).

FIG. 15 shows the effect of CAR-iNKT cells of the invention on HTLV-1 expressing CD4+ T cell. The frequency of HTLV-1 Tax expressing CD4+ cells after co-culture with CAR-iNKT (see FIGS. 15A and 15B). FIG. 15C shows the frequency of TCRVβ1 and TCRVβ2+ CD3+ T cells after co-culture. CellTraceViolet stained, positively selected CD4+ cells from the same donors were cultured alone or in the presence of Vβ1- or CD19-CAR iNKT. After 16-18h, the cells were stained with a viability stain, anti-CD3, -CD4, -CD8 and -TCRVβ1. The cells were subsequently fixed, and stained intracellularly with anti-Tax antibody. The cells were then analysed by flow cytometry and the frequency of live TCRVβ1+ or TCRVβ2+CD3+ cells and live Tax+CD4+ cells was determined.

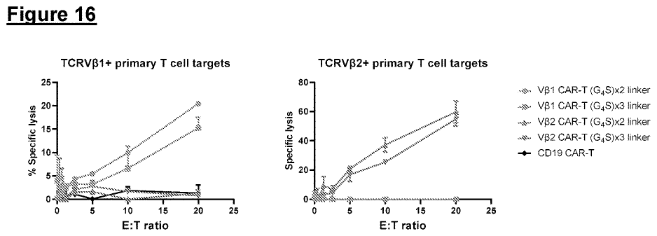

FIG. 16 provides a comparison of the effect of linker size on CAR-T efficacy. Killing of in-vitro expanded autologous primary T cell lines by anti-TCR CAR-T cells and anti-CD19 CAR-T. Target cells were stained with CFSE co-cultured with effectors at a range of effector to target (E:T) ratios in duplicate. After 24 h cells were harvested and stained with 7aad. The frequency of dead targets in each culture condition was assayed by flow cytometry.

### EXAMPLES

As discussed below, the inventors have generated exemplary anti-TCRVβ CAR constructs (referred herein as clone BL37.2 for TCR Vβ1, MPB2D5 for TCR Vβ2, FIN9 for TCR Vβ9 and C21 for TCR Vβ11) (see Example 1) and tested their activities against primary T cells and cancer T-cell lines specifically expressing a selected TCRVB (see Example 2). Subsequently, the inventors have generated effector T cells expressing the selected anti-TCRVβ CAR constructs, in particular, iNKT cells expressing the selected anti-TCRVβ CAR constructs, and have analysed their suitability for immunotherapy of T-cell malignancies (see Example 3). Then, the inventors have successfully demonstrated that the anti-TCRVβ CAR-INKT cells are highly active against ATL (see Example 4) and do not impair CTL immunity against viral antigen, nor do they promote replication activity of HTLV-1, thereby exhibiting an important safety feature required for the treatment of ATL and TCL.

### Materials and Methods

### Ethics Statement

Patients with ATL attended the National Centre for Human Retrovirology (Imperial College Healthcare NHS Trust, St Mary's Hospital, London), where written informed consent was obtained. Research involving samples from patients with ATL was conducted under the governance of the Communicable Diseases Research Group Tissue Bank, approved by the UK National Research Ethics Service (09/H0606/106, 15/SC/0089, 20/SC/0226).

### Cell Lines and Chemicals

HEK 293T were passaged twice a week by trypsinisation and maintained at 30-70% confluency in complete Dulbecco's modified eagle medium (DMEM) with 10% (v/v) Foetal bovine serum (FBS), 100 units/ml penicillin, 100 μg/mL streptomycin and 2 mM L-glutamine. JRT3-T3.5 were obtained from the ATCC and maintained at 1×10⁵-1×10⁶ cells/ml in complete RPMI medium with 10% (v/v) FBS, 100 units/ml penicillin, 100 μg/mL streptomycin and 2 mM L-glutamine or iNKT medium (RPMI with 10% FBS, 15 mM Hepes, 1 mM Sodium Pyruvate, 1×MEM nonessential amino acids, 4 mM L-Glutamine, 0.05 mM beta-mercaptoethanol, 100 units/ml penicillin and 100 μg/mL streptomycin). C1R-CD1d cells (a gift from Vincenzo Cerundolo, Oxford University) were maintained at 1×10⁵-1×10⁶ cells/ml in complete RPMI.

### Generation of CAR Constructs

Four monoclonal antibodies were identified for proof of principle studies targeting each of TCR Vβ1 (clone BL37.2), Vβ2 (MPB2D5), Vβ9 (FIN9) and Vβ11 (C21) purchased unconjugated from Beckman Coulter. The amino acid sequence of the variable region of the heavy and light chains of BL37.2, MPB2D5 and FIN9 were determined by mass spectrometry (Rapid Novor, Canada). For clone C21, total RNA was extracted from hybridoma cells, reverse transcribed, and heavy and light chain fragments were amplified using isotype-specific primers. Amplified fragments were cloned into a standard cloning vector, transformed into E. coli, and five colonies from each region of interest were sequenced by Sanger sequencing (GenScript). Codon optimised gene fragments were synthesised by Genewiz and cloned into a lentiviral expression vector to generate the construct shown in FIGS. 1 and 12.

### Production of Lentiviruses

HEK293T cells were seeded at a density of 4×10⁶/10 cm plate in 10 ml complete DMEM. After 24 h, the cells were transfected with lentiviral expression vectors and second-generation packaging plasmids using GeneJuice (from Merck). Supernatants were collected 48 h post-transfection and clarified by centrifugation at 500 g for 5 minutes, and then passed through a 0.45 μM filter. Lentiviral particles were concentrated by ultracentrifugation at 23,000 rpm for 2 h at 4 °C, resuspended in 200-300 μl serum-free RPMI and stored at −80 °C until use. The viral titre was determined by titration using HEK-293T cells.

### Isolation of Peripheral Blood Mononuclear Cells

Peripheral blood mononuclear cells (PBMCs) were purified from apheresis cones or EDTA anticoagulated blood by density gradient centrifugation. Briefly, cells were harvested from the cones and diluted in PBS up to 100 ml. 25 ml diluted cells were layered over 15 ml histopaque (Sigma) and centrifuged at 800 g for 20 minutes. The buffy coat was harvested, washed with PBS (400 g, 5 min at RT) and cryopreserved in FBS with 10% dimethyl sulfoxide (DMSO) until use.

Lentiviral Transduction of T and iNKT Cells

PBMCs were thawed rapidly, washed twice in RPMI with 10% FBS and resuspended in a small volume of media. iNKT were positively selected using magnetic bead separation according to the manufacturer's instructions (Miltenyi Biotech), passing the cells over two consecutive LS columns. The purity of the positive fraction was evaluated by flow cytometric staining using a fixable viability stain (Live/Dead Near-Infrared, Life Sciences) and anti-TCRVα24-Jα18 BV421 (clone 6B11), -TCRVβ11 APC, -CD3 BV510, -CD4 BV605, -CD8 FITC. For iNKT cultures, the positively selected fraction was placed in culture with a 1:1 ratio of irradiated (35 Gy) autologous feeder cells prepared from the negative fraction. For T cell cultures, the iNKT-depleted flow through fraction was placed in culture alone. T and iNKT cells were cultured in iNKT media and stimulated with 50 ng/ml anti-CD3 and 50 ng/ml anti-CD28 (Miltenyi-Biotech) and 150 IU/ml IL-15. After 48 h culture, the required volume of each lentivirus was placed in retronectin-coated plates to give a multiplicity of infection (MOI) of 2.5-5 infectious units per cell. Cells were harvested, counted and added to the transduction plate, then centrifuged at 1000 g for 40 min at 32 °C. On day 7 of iNKT cultures, CD1d-expressing feeder cells were loaded with 200 ng/ml α-Galactosylceramide (αGalCer, BioVision) for 2-4 h, irradiated and added to the culture at a ratio of 1:1. Cells were fed twice weekly with fresh media containing 150 IU/ml IL-15.

### Quantification of Lentiviral Transduction Efficiency

Transduction efficiency was evaluated from 5 days post-transduction. Cells were washed twice in cold PBS, incubated with 1 μg/ml streptavidin protein L (Pierce) for 45 min at 4 °C. Cells were washed twice with PBS, stained with Live/dead near-infrared for 5 min at RT, washed with PBS 0.5% FBS and stained for 20 min at RT with streptavidin-PE/BV421 and anti-CD3, -CD4, -CD8, -TCRVβ11 and -TCRVα24-Jα18. Cells were acquired the same day using a BD Fortessa and analysed using Kaluza (Beckman Coulter).

### Isolation and Culture of Primary T Cell Targets

Autologous primary T cell lines expressing TCRVβ subunits of interest were established by magnetic enrichment and expansion in vitro. PBMC were stained for 20 min with PE-conjugated anti-TCRVβ antibodies (Beckman Coulter) at RT, washed once in complete RPMI and incubated for a further 20 minutes at 4 °C with anti-PE microbeads (Miltenyi Biotech). After one further wash, the cells were passed over two consecutive LS columns. The positive fraction was placed in culture with anti-CD3, anti-CD28 and 150 IU/ml IL-2.

### Generation of GFP-TCR—Expressing JRT3-T3.5 Cells

TCR β-chain sequences which contained Vβ subunits of interest were identified in publicly available databases. The Table below summarises TCR gene (TRBV) and protein (TCRVβ) nomenclature, and specifies the proteins for which monoclonal antibodies are commercially available. The codon optimised nucleotide sequence corresponding to the full-length beta chain was synthesised (using Genewiz) and cloned into a third-generation lentiviral expression vector (LeGO-iG2).

TABLE 2
TCR gene (TRBV) and protein (TCRVβ) nomenclature, including proteins
for which monoclonal antibodies are commercially available.

| | | Reagents monoclonal antibodies | | |
|---|---|---|---|---|
| TRBV | TRBV | mAb clone name | | % of peripheral |
| gene name | allele name | and Specificity | Protein name | CD3 positive |
| TRBV2 | TRBV2*01 | IMMU 546 (TRBV2) | TCRVβ22 | 2.4-5.1 |
| | TRBV2*02 | | | |
| | TR8V2*03 | | | |
| TRBV3-1 | TRBV3-1*01 | FIN9 (TRBV3-1)/AMKB1-2 | TCRVβ9 | 0.5-3 |
| | TRBV3-1*02 | (TRBV3-1) | | |
| TRBV4-1 | TRBV4-1*01 | ZOE (TRBV4-1, TRBV4-2, | TCRVβ7.1 | 1.5-2.4 |
| | TRBV4-1*02 | TRBV4-3)/3G5 (TRBV4-1) | | |
| TRBV4-2 | TRBV4-2*01 | ZOE (TRBV4-1, TRBV4-2, | TCRVβ7.1 | |
| | TRBV4-2*02 | TRBV4-3) | | |
| TRBV4-3 | TRBV4-3*01 | ZOE (TRBV4-1, TRBV4-2, | TCRVβ7.1 | |
| | TRBV4-3*02 | TRBV4-3) | | |
| | TRBV4-3*03 | | | |
| | TRBV4-3*04 | ZIZOU4 (TRBV4-3) | TCRVβ7.2 | |
| TRBV5-1 | TRBV5-1*01 | IMMU157 (TRBV5-1) | TCRVβ5.1 | 4.2-7.2 |
| | TRBV5-1*02 | LC4 (TRBV5-1) | TCRVβ5.1 | 0.5-3 |
| TRBV5-4 | TRBV5-1*01 | no antibody commercially available | n/a | n/a |
| | TRBV5-1*02 | | | |
| | TRBV5-1*03 | | | |
| | TRBV5-1*04 | | | |
| TRBV5-5 | TRBV5-5*01 | 3D11 (TRBV5-5)/1C1 (TRBV5-5, | TCRVβ5.2/5.3 | 0.5-3 |
| | TRBV5-5*02 | TRBV5-6)/W112 (TRBV5-5)/MH3-2 | | |
| | TRBV5-5*03 | (TRBV5-5, TRBV5-6) | | |
| TRBV5-6 | TRBV5-6*01 | 36213 (TRBV5-6)/1C1 (TRBV5-5, | TCRVβ5.2 | 0.5-3 |
| | | TRBV5-6)/MH3-2 (TRBV5-5, | | |
| | | TRBV5-6) | | |
| TRBV5-8 | TRBV5-8*01 | no antibody commercially available | n/a | n/a |
| | TRBV5-8*02 | | | |
| TRBV6-1 | TRBV6-1*01 | BAM13 (TRBV6-1, TRBV6-5) | TCRVβ13.1, 13.3 | 3-6 |
| TRBV6-2 | TRBV6-2*01 | H132 | TCRVβ13.2 | |
| TRBV6-3 | TRBV6-3*01 | no antibody commercially available | n/a | n/a |
| TRBV6-4 | TRBV6-4*01 | no antibody commercially available | n/a | n/a |
| | TRBV6-4*02 | | | |
| TRBV6-5 | TRBV6-5*01 | IMMU 222 (TRBV6-5, TRBV6-6 | TCRVβ13.1, 13.3 | 1.8-3.3 |
| | | and TRBV6-9)/BAM13 (TRBV6-1, | | |
| | | TRBV6-5) | | |
| TRBV6-6 | TRBV6-6*01 | JU-74 (TRBV6-6)/JU74.3 (TRBV6-6) | TCRVβ13.6 | 3-6 |
| | TRBV6-6*02 | | | |
| | TRBV6-6*03 | | | |
| | TRBV6-6*04 | IMMU 222 (TRBV6-5, TRBV6-6 | TCRVβ13.1 | 3-6 |
| | TRBV6-6*05 | and TRBV6-9) | | |
| TRBV6-8 | TRBV6-8*01 | no antibody commercially available | n/a | n/a |
| TRBV6-9 | TRBV6-9*01 | IMMU 222 (TRBV6-5, TRBV6-6 | TCRVβ13.1 | 3-6 |
| | | and TRBV6-9) | | |
| TRBV7-2 | TRBV7-2*01 | OT145 (TRBV7-2) | TCRVβ6.7 | 0.5-3 |
| | TRBV7-2*02 | | | |
| | TRBV7-2*03 | | | |
| | TRBV7-2*04 | | | |
| TRBV7-3 | TRBV7-3*01 | no antibody commercially available | n/a | n/a |
| | TRBV7-3*02 | | | |
| | TRBV7-3*03 | | | |
| TRBV7-4 | TRBV7-4*01 | no antibody commercially available | n/a | n/a |
| TRBV7-6 | TRBV7-6*01 | no antibody commercially available | n/a | n/a |
| | TRBV7-6*02 | | | |
| TRBV7-7 | TRBV7-7*01 | no antibody commercially available | n/a | n/a |
| | TRBV7-7*02 | | | |
| TRBV7-8 | TRBV7-8*01 | no antibody commercially available | n/a | n/a |
| | TRBV7-8*02 | | | |
| | TRBV7-8*03 | | | |
| TRBV7-9 | TRBV7-9*01 | no antibody commercially available | n/a | n/a |
| | TRBV7-9*02 | | | |
| | TRBV7-9*03 | | | |
| | TRBV7-9*04 | | | |
| | TRBV7-9*05 | | | |
| | TRBV7-9*06 | | | |
| | TRBV7-9*07 | | | |
| TRBV9 | TRBV9*01 | BL37.2 (TRBV9) | TCRVβ1 | 2.4-6.6 |
| | TRBV9*02 | | | |
| | TRBV9*03 | | | |
| TRBV10-1 | TRBV10-1*01 | S511 (TRBV10-1, TRBV10-2, | TCRVβ12 | 0.5-3 |
| | TRBV10-1*02 | TRBV10-3) | | |
| TRBV10-2 | TRBV10-2*01 | S511 (TRBV10-1, TRBV10-2, | TCRVβ12 | 0.5-3 |
| | TRBV10-2*02 | TRBV10-3) | | |
| TRBV10-3 | TRBV10-3*01 | VER2.32.1 (TRBV10-3) | TCRVβ12 | 1.5 |
| | TRBV10-3*02 | | | |
| | TRBV10-3*03 | S511 (TRBV10-1, TRBV10-2, | TCRVβ12 | 0.5-3 |
| | TRBV10-3*04 | TRBV10-3) | | |
| TRBV11-1 | TRBV11-1*01 | no antibody commercially available | n/a | n/a |
| TRBV11-2 | TRBV11-2*01 | IG125 (TRBV11-2) | TCRVβ21.3 | 2.2-3.6 |
| | TRBV11-2*02 | | | |
| | TRBV11-2*03 | | | |
| TRBV11-3 | TRBV11-3*01 | no antibody commercially available | n/a | n/a |
| | TRBV11-3*02 | | | |
| | TRBV11-3*03 | | | |
| | TRBV11-3*04 | | | |
| TRBV12-3 | TRBV12-3*01 | 56C5 (TRBV12-3, TRBV12-4)/56C5.2 | TCRVβ8.1/8.2 | 2.6-5.1 |
| TRBV12-4 | TRBV12-4*01 | (TRBV12-3, TRBV12-4)/16G8 (TRBV12-3, | | |
| | TRBV12-4*02 | TRBV12-4)/MX-6 (TRBV12-3, TRBV12-4) | | |
| TRBV12-5 | TRBV12-5*01 | JR2 (TRBV12-3, TRBV12-4, TRBV12-5) | TCRVβ8 | 0.5-3 |
| TRBV13 | TRBV13*01 | AF-23 (TRBV13)/AF23 | TCRVβ23 | 0.4-5 |
| | TRBV13*02 | (TRBV13)/AHUT7 (Vbeta23) | | |
| TRBV14 | TRBV14*01 | TAMAYA1.2 (TRBV14) | TCRVβ16 | 0.8 |
| | TRBV14*02 | | | |
| TRBV15 | TRBV15*01 | no antibody commercially available | n/a | n/a |
| | TRBV15*02 | | | |
| | TRBV15*03 | | | |
| TRBV16 | TRBV16*01 | no antibody commercially available | n/a | n/a |
| | TRBV16*02 | | | |
| TRBV18 | TRBV18*01 | BA62 (TRBV18)/BA62.6 (TRBV18) | TCRVβ18 | 0.7-2.2 |
| TRBV19 | TRBV19*01 | C1 (TRBV19)/E17.5F3 | TCRVβ17 | 0.5-7 |
| | TRBV19*02 | (TRBV19)/E17.5F3.15.13 | | |
| | TRBV19*03 | (TRBV19) | | |
| TRBV20-1 | TRBV20-1*01 | MPB2D5 (TRBV20-1) | TCRVβ2 | 4.2-9.1 |
| | TRBV20-1*02 | | | |
| | TRBV20-1*03 | | | |
| | TRBV20-1*04 | | | |
| | TRBV20-1*05 | | | |
| | TRBV20-1*06 | | | |
| | TRBV20-1*07 | | | |
| TRBV24-1 | TRBV24-1*01 | no antibody commercially available | n/a | n/a |
| TRBV25-1 | TRBV25-1*01 | C21 (TRBV25-1) | TCRVβ11 | 0.8 |
| TRBV27 | TRBV27*01 | CAS1.1.3 (TRBV27) | TCRVβ14 | 2.2-5.6 |
| TRBV28 | TRBV28*01 | CH92 (TRBV28)/8F10 | TCRVβ3/3.1 | 2.9 |
| | | (TRBV28)/JOVI-3 (TRBV28) | | |
| TRBV29-1 | TRBV29-1*01 | WJF24 | TCRVβ4 | |
| | TRBV29-1*02 | | | |
| | TRBV29-1*03 | | | |
| TRBV30 | TRBV30*01 | ELL1.4 (TRBV30) | TCRVβ20 | 2.2 |
| | TRBV30*02 | | | |
| | TRBV30*03 | | | |
| | TRBV30*04 | | | |

### 7aad Cytotoxicity Assay

One day before the assessment of cytotoxicity, effector cells were counted and fed with fresh media. Target cells were washed twice with PBS, incubated in 0.5 μM CFSE/Cell trace violet for 10 min at 37 °C, and washed twice in iNKT media and placed back in culture with or without αGalCer as required. On the day of the assay, cells were centrifuged, resuspended in iNKT media with 15 IU/ml IL-15. Cells were mixed to achieve a range of effector:target (E:T) ratios, placed in duplicate in U bottomed 96 well plates, centrifuged for 1 min at 100 g. Cells were cultured for 4-24h, centrifuged for 3 min at 800 g and resuspended in PBS with 7aad (5 μg/ml). After 20 min incubation, each well was washed with 150 μl PBS, centrifuged for 3 min at 800 g and resuspended in 100 μl PBS. In each well, the frequency of dead (7AAD+) target cells was enumerated by flow cytometry.

### Fresh PBMC Cytotoxicity Assay

One day before the assessment of cytotoxicity, effector cells were counted and fed with fresh media. On the day of the assay, cryopreserved PBMC were thawed were washed (400 g for 5 min) once with iNKT media, once with PBS, incubated in 0.5 μM CFSE/Cell trace violet for 10 min at 37 °C, and washed twice in iNKT media. PBMC were placed in a U bottomed 96 well plate with 15 IU/ml IL-15 with and without CAR-iNKT cells at a range of effector:target ratios in triplicate. The plate was centrifuged for 1 min at 100 g and cultured for 24 h. Wells were washed with 150 μl PBS (800 g 3 min), stained with Live/dead near-infrared for 5 min at RT, washed with FACS buffer (PBS 7% (v/v) NGS) and stained with anti-TCRVβ-PE, -TCRαβ-FITC, -CD4-BV605, -CCR4-APC and -CD26-PeCy7 for 20 min at RT in FACS buffer. Cells were washed with 150 μl FACS buffer, fixed for 20 min with 150 μl fixation buffer (Biolegend) and washed once more with FACS buffer before storing at 4 °C until acquisition.

### Intracellular Cytokine Staining and Degranulation Assay

Twenty four hours (24 h) before evaluating intracellular cytokine production/degranulation, effector cells were fed, and target cells were stained with CFSE. On the day of the experiment, target and effector cells were harvested, counted and mixed to give a 1:1 E:T ratio in iNKT media containing 20 μg/ml DNase, 15 IU/ml IL-15, 10.6 μM brefeldin, 2 μM monensin (1× protein transport inhibitor, eBioscience) and 2.5 μl anti-CD107a-BV421 (clone H4A3)/200 μl culture well. Cells were cultured for 6 h, centrifuged at 800 g for 3 minutes to remove the culture supernatant and resuspended in live/dead NIR viability stain. After 5 minutes incubation at RT, 150 μl PBS 0.5% FBS was added to each well, and the plate was washed again. Cells were resuspended in 150 μl Fixation/permeabilisation buffer (eBiosciences FoxP3 buffer set, Life Technologies) incubated for 30 min at RT and washed once in Permeabilisation buffer. Cells were stained with anti-CD3-BV510 (clone UCHT-1) -CD4-BV605 (RPA-T4) -CD8-AF700 (RPA-T8) -IFN-γ-BV711 (4S.B3), -TNF-α-PeCy7 (Mab11), Granzyme B-PE (QA16A02) and Perforin-APC (B-D48) diluted in permeabilisation buffer. After 30 min incubation at RT, cells were washed once in 150 μl permeabilisation buffer and resuspended in PBS 0.5% FBS until acquisition.

### Pentamer Staining

HTLV-1-infected CD4+ cells express viral antigens on short term culture ex vivo, and thus could present the Tax11-19 peptide in the context of HLA-A*0201. Thus, to minimise the possibility of downregulation of the cognate TCR on CTL due to antigen encounter during the culture period, CD4+ T cells were depleted from PBMCs from HLA-A*0201+ HTLV-1 carriers using anti-CD4 PE and anti-PE microbeads as described above. CD4-depleted PBMCs were stained with cell-trace violet. The positive and negative fractions were cultured in iNKT media with 15 IU/ml IL-15 in the presence or absence of CAR-INKT cells. After 16-18 h co-culture, cells were stained with live/dead near-infrared as described above and resuspended in 40 μl PBS with 10 μl HTLV-1 Tax11-19 or Influenza A M158-66 Pentamer-APC for 10 minutes, after which anti-CD3-BV510 and -CD8-AF700 were added. After a further 20 min incubation at RT, cells were washed and fixed for 30 minutes by resuspending cells in 150 μl with Fixation buffer (Biolegend). After one wash with PBS, the frequency of live pentamer positive Cell trace violet+ CD8+ T cells in each culture was assessed by flow cytometry. Similarly, the CD4+ fraction was stained with live/dead, anti-CD4-BV605, -CD3-BV510, washed with PBS 7% NGS and fixed for 30 minutes with 150 μl ebioscience FoxP3 fixation/permeabilisation buffer. Cells were then washed once in ebioscience permeabilisation buffer, and stained intracellularly with anti-Tax AF647 for 30 minutes. Cells were washed once more with 150 μl permeabilisation buffer, resuspended in PBS and stored at 4 °C in the dark until acquisition. The number of Tax+CD4+Cell trace violet+ cells was measured by flow cytometry.

### In Vivo Experiment

5×10⁶ TCRVβ+ JRT cells were suspended in matrigel and injected subcutaneously to the flank of NSG mice. On day 18, 1×10⁶ effector cells were injected intravenously into the tail vein. Groups consisted of untreated (n=5), Vβ2 CAR-iNKT (n=7) or CD19 CAR-INKT (n=5). Tumour volume for each group was measured periodically using calipers. At the end of the experiment, tumours were excised and weighed.

### Off-Target Killing Assay

PBMC from two normal donors were cultured alone or transduced with lentivirus which encoded Vβ1, Vβ2, Vβ9 and Vβ11-specific CAR constructs. Five days post transduction, transduction efficiency was evaluated by protein L staining, and the frequency of CD3+ cells expressing each of 24 TCRVβ subunits was quantified. Values were reported as mean percentage of cells expressing each subunit normalised to the frequency of cells expressing that subunit in the untransduced PBMC control from each individual.

### Results

### Example 1: Generation of CAR Constructs Targeting TCRVβ1, -2, -9 and -11

The inventors generated four lentiviral CAR constructs to target TCRVβ1 (expressed on 3.5% of CD3+ T cells in healthy donors), TCRVβ2 (8.3%), TCRVβ9 (3.2%) and TCRVβ11 (1%). The anti-TCRVβ1 CAR is called the “BL37.2 CAR”, the anti-TCRVβ2 CAR is called the “MPB2D5 CAR”, the anti-TCRVβ9 CAR is called the “FIN9 CAR”, and the anti-TCRVβ11 CAR is called the “C21 CAR”.

Since hybridoma clones were not available for the first three mAbs, the inventors employed reverse engineering of CARs after determining the amino acid sequence of mAbs by mass spectrometry. The corresponding nucleotide sequence was codon optimised for expression in human cells, and used to construct second-generation CARs in which the ectodomain comprises CD8α leader peptide-VL-(GGGGS)₂ linker-VH-CD8α hinge-CD8α transmembrane domain, while the endodomain is composed of a portion of the CD8α cytoplasmic domain and the signalling (i.e. stimulatory and co-stimulatory) domains of CD28-CD3ζ (see FIGS. 1 and 2).

Referring to FIGS. 1 and 2, there is shown a schematic map of one embodiment of a coding sequence of an expression vector for an anti-TCRVβ CAR lentiviral CAR of the invention. It will be appreciated that the vector (DNA) also corresponds to the CAR (protein). The CAR includes a human CD8α signal peptide, an antigen binding domain comprising a VL, flexible linker and VH, a CD8α hinge domain comprising a transmembrane domain and cytoplasmic domain, and an intracellular signalling domain comprising a CD28 co-stimulatory domain and CD3ζ stimulatory domain.

To generate an anti-TCRVβ11 CAR with codon optimised VH/VL sequence, the inventors amplified the expressed VH and VL chains using mRNA extracted from the hybridoma C21. The resulting CAR constructs are represented in FIG. 3. For each of the four types of CAR (i.e. “BL37.2 CAR”, “MPB2D5 CAR”, “FIN9 CAR”, and “C21 CAR”), a construct with 2×G₄S linker and a construct with a 3×G₄S linker, respectively, was created and is illustrated. The position of each of the components of the construct (5′ to 3′) follows the order disclosed in the schematic map of FIG. 1.

The signal peptide is identical to that of human CD8α. As the signal peptide matches the species of the effector cells (as described later), and as CD8α is highly expressed in T cells, the inventors believe that their signal peptide will provide optimal expression of the CAR construct in the effector cells. The hinge region of the CAR contains amino acids 128-210 of human CD8α, which includes 55 amino acids from the extracellular domain, the full transmembrane helical domain and 7 amino acids of the cytoplasmic domain. The intracellular domain of the CAR includes the CD3ζ stimulatory domain and the CD28 co-stimulatory domain.

Plasmids encoding the lentiviral CAR constructs targeting TCRVβ1, -2, -9 and -11 are shown in FIGS. 4, 5, 6 and 7, respectively.

### Example 2: Assessment of the Specificity, Cytokine Production and Cytotoxic Activity of Effector Cell Expressing the Anti-TCRVβ CAR Construct

To begin testing the activity and specificity of CARs described in Example 1, the inventors transduced them into PBMC T cells of two healthy donors though lentivirus transduction. Five days post-transduction, CAR expression on T cells was over 50% (FIGS. 8A and 8B). Simultaneously, with reference to untransduced T cells, the inventors quantified the frequency of T cells expressing each of 24 TCRVβ chains in CAR-transduced PBMC. The inventors found that T cells expressing TCRVβ1, Vβ2, Vβ9 and Vβ11 were almost entirely depleted from PBMCs transduced with the respective cognate CAR construct (median 98% reduction; range 90-100%) (FIG. 8C, and FIG. 12). In contrast, the inventors observed a 4% increase in the median frequency of untargeted Vβ subunits in transduced cultures relative to the untransduced control indicating lack of ‘off-target; killing by TCRVβ family-specific CAR-T cells.

In summary, anti-TCRVβ1, -2, -9 and -11 CAR are robustly expressed, active and selective against their cognate TCRVβ chain targets.

### Example 3: Anti-TCRVβ Chain CAR Against Primary and Cancer T Cell Lines

To further investigate the anti-T cell activity and TCRVB specificity of the four CAR constructs, the inventors first tested TCRVβ CAR-T cells against expanded autologous primary T cell lines highly purified to express TCRVβ chains of interest (FIG. 13A). The inventors found that all four anti-TCRVβ CAR-T cells selectively killed their cognate T cell line. However, the level of cytotoxicity differed and reflected the intensity of staining with the corresponding mAb, with both being highest for anti-TCRVβ1 and 2 lowest for TCRVβ9 (FIG. 8D).

Next, the inventors engineered the JRT3-T3.5 T cell line to express the TCRVβ1 or 2 chains as targets. JRT3-T3.5 is a derivative of the Jurkat T cell line, itself derived from a patient with TCRVβ+ lymphoblastic T cell lymphoma. Due to deletion of the endogenous TCRVβ chain gene, JRT3-T3.5 cells lack expression of TCR as well as of CD3 and, therefore, as expected, introduction of an exogenous TCRVB cDNA restored expression of both (FIG. 13B). Accordingly, the inventors found that anti-TCRVβ1 and two CAR-T cells kill JRT3-T3.5 cells expressing their cognate target TCRVB, but not the parental cell line (FIG. 8E).

In a complementary functional approach, the inventors found that upon intracellular staining, anti-TCRVβ2 CAR-T cells expressed IFNg, TNFa and CD107a (denoting cytotoxic degranulation) when co-cultured with TCRVβ2-expressing JRT3-T3.5 cells or primary T cell line, but not when cultured in the presence of the parental JRT3-T3.5 or alone, with more than 30% of CD4+ and CD8+ CAR-T cells being poly-functional, i.e. co-expressing at least two molecules (FIGS. 8E and 8F).

Together, these findings show that the anti-TCRVβ CARs of the invention, which have been developed by reverse engineering are highly specific and active in vitro.

### Example 4: Anti-TCRVβ CAR-iNKT Cells for ‘Off-the-Shelf’ Immunotherapy of T Cell Malignancies

The feasibility of deploying iNKT cells as effector cells for CAR immunotherapy of blood cancers has been demonstrated by earlier studies. Therefore, using their own established manufacturing protocol, the inventors have generated anti-TCRVβ1, 2, 9 & 11 CAR-iNKT cells. Anti-TCRVβ1, 2 & 9 CAR-INKT cells efficiently killed JRT3-T3.5 as well as primary T cell lines expressing the corresponding TCRβ chain (FIGS. 9A and 9B) while CD4+ and CD4− CAR-iNKT cells specifically expressed or co-expressed IFNg, TNFa and CD107a upon stimulation with TCRVβ2-expressing JRT3-T3.5 and primary T cells (FIG. 9C).

Since JRT3-T3.5 cells express CD1d (FIG. 14A), the inventors tested whether reactivity of anti-TCRVβ CAR-iNKT cell could be enhanced in the presence of aGC, a glycolipid ligand of CD1d that powerfully and selectively activates iNKT-cells^{16,17}. The inventors found that while anti-TCRVβ1&2 CAR-T cytotoxicity against parental and cognate TCRVβ-expressing JRT3-T3.5 cells was not enhanced in the presence of aGC, cytotoxicity of anti-TCRVβ1&2 CAR-iNKT was enhanced against both targets and more so against the parental JRT3-T3.5 T cells (FIGS. 9D and 9E) thus indicating the functional relevance of iTCR in the anti-tumour activity of CAR-iNKT cells.

The inventors further tested the activity of TCRVβ CAR-INKT cells in an in vivo model of T cell lymphoma in which TCRVβ2-expressing JRT3-T3.5 T cells were injected subcutaneously to the flank of NSG mice (FIG. 9F). After tumour engraftment, mice were left untreated or treated with iv transfer of 10⁶ TCRVβ2 or CD19 CAR-iNKT cells per mouse. Consistent with lack of expression of CD19 by JRT3-T3.5 T cells, CD19 CAR-iNKT failed to impact the growth of T cell lymphoma tumours; by contrast, TCRVβ2 CAR-iNKT cells significantly inhibited tumour volume and weight (FIGS. 9G and 9H).

Finally, CAR-iNKT cell-treated mice showed no evidence of excess weight loss or other clinical signs of aGVHD after 42 days of monitoring (FIG. 14B), thus supporting the notion of using allogeneic iNKT cells as an ‘off-the-shelf’ platform for immunotherapy of cancer without risk of aGVHD.

Together these data demonstrate the therapeutic potential of the allogeneic TCRVB CAR-iNKT cells of the invention for the treatment of T cell lymphomas.

### Example 5: Anti-TCRVβ CAR-iNKT Cells are Active Against ATL

Similar to the test conducted on JRT3-T3.5 T cell lymphoma cells, the inventors also tested the reactivity of allogeneic anti-TCRVβ1&2 CAR-iNKT cells against PBMC from a normal donor and against PBMCs from two ATL patients enriched in lymphoma cells clonally expressing TCRVβ1 or TCRVβ2 (FIG. 10A). In addition to their clonal TCR, as the inventors previously demonstrated, ATL malignant cells also co-express CD4 and CCR4 and are negative for CD26 (FIG. 10A). Therefore, the inventors co-culture matched anti-TCRβ CAR-iNKT and anti-CD19 CAR-iNKT with PBMC from each ATL and normal donor at a range of E: T ratios. Because co-culture with the anti-TCRVB CAR-iNKT cells could block subsequent staining with the same anti-TCRVβ antibody clone, as the inventors previously reported, the inventors used the immunophenotype CD4+CCR4^{med/hi}CD26− to identify the lymphoma/leukaemia cells in PBMC from patients with ATL. The inventors found that in patients 1 and 2, CCR4+CD26− cells comprised 90% of CD4+ cells and nearly all expressed TCRVβ1 or TCRVβ2 respectively while in the normal donor CCR4+CD26− cells comprised 25% of CD4+ cells and TCRVβ1 and TCRVβ2 comprised less than 8%.

At an Effector:CD4+ T cell ratio of 1:1, anti-TCRVβ1 CAR-INKT killed 75% of CD4+CCR4+CD26− cells in patient 1, and 10% of the ‘rest’ (i.e., CCR4−CD26−) of CD4+ cells. At the same ratio in patient 2, anti-TCRVβ2 CAR-INKT killed 54% of CD4+CCR4+CD26− cells. The frequency of CD4+ cells expressing other TCRVB subunits was too low to determine whether there was no off-target killing of normal CD4+ cells in this patient. Minimal killing of CD4+CCR4+CD26− cells and ‘other CD4+’ cells was observed when PBMC from patients or the normal donor were co-cultured with anti-CD19 CAR-iNKT cells. Similarly, minimal killing was observed after PBMC from the normal donor were co-cultured with anti-TCRVβ1 and -TCRVβ2 CAR-INKT cells, likely because of the low frequency of target cells present in the populations analysed (FIGS. 10B and 10C).

In conclusion, anti-TCRVβ CAR-iNKT cells are highly active and specific against primary ATL cancer cells.

### Example 6: Effect of Anti-Vβ CAR-iNKT on Antiviral CTL Immunity and HTLV-1 Viral Status

In order to further demonstrate selective targeting, the inventors studied the in-vitro impact of the anti-TCRVβ CAR-INKT cells of the invention on antiviral CD8+ T cells in HTVL-1 infected individuals. For this purpose, the inventors cultured PBMC from three HLA-A*0201+ HTLV-1 infected individuals (i.e., carrier) alone or in the presence of CAR-iNKT cells and evaluated the frequency of T cells which bound HLA-A*0201 HTLV-1 Tax11-19 or Influenza A M158-66 peptide-MHC pentamers (FIGS. 11A and 11B). When cultured alone, the frequency of Influenza A M158-66 pentamer+ CD3+ T cells ranged from 0.14-0.23% of CD3+ cells, and the frequency of HTLV-1 Tax11-19 pentamer+ CD3+ T cells ranged from 0.23-0.88%. When co-cultured with Vβ1-, Vβ2- or CD19-CAR-iNKT cells, there was no change in the frequency of pentamer+ cells (FIGS. 11A and 11B). Efficient depletion of T cells expressing TCRVβ1 and -β2 by their cognate CAR-iNKT ells was confirmed in parallel cultures from the same donors (FIG. 15C).

Since killing of target cells by CAR-iNKT cells is associated with secretion of inflammatory cytokines, the inventors asked whether killing by CAR-iNKT would enhance spontaneous expression of HTLV-1 Tax, a process that might facilitate HTLV-1 infection of non-infected CD4+ T cells. For this purpose, positively selected CD4+ T cells from the same three HTLV-1 infected individuals were first cultured alone or in the presence of anti-Vβ1, -Vβ2 and -CD19 CAR-iNKT cells for 18 hrs followed by evaluation by intracellular staining of the frequency of Tax-expressing CD4+ T cells. Despite clear evidence of CAR-mediated depletion of T cells expressing the target TCRVβ molecules, there was no change in the frequency of Tax+CD4+ T cells in any of the CAR-iNKT co-cultures when compared with cells cultured alone (FIGS. 11C and 11D, FIGS. 15A and 15B).

Therefore, CAR-iNKT cells targeting TCRVβ chains do not impair CTL immunity against viral antigens, neither do their promote replication activity of HTLV-1, both important considerations for the safe treatment of TCL and ATL.

### Example 7: Comparison of CAR Constructs with (G4S)2 and (G4S)3 Linkers

In order to compare the effect of the linker length on CAR efficiency in vitro, PBMCT cells were transduced with TCRVβ1 and Vβ2 CARs which contained either the (G4S)2 or (G4S)3 linkers and co-cultured with primary T cell target cells which expressed either TCRVβ1 or TCRVβ2.

### Discussion

The present invention demonstrates that anti-TCRVβ CAR immunotherapy can be developed as a sensitive, specific and highly effective strategy for the treatment of TCRVβ-expressing T cell malignancies. Given that the distribution of the TCRVB repertoire in T cell malignancies is the same as in normal T cells, approximately 15% of TCL can be targeted by the CARs developed and tested here, thus paving the way for development of CARs against all TCRVβ families for which mAbs are available (currently for roughly 70% of TCRβ chain families) or can be developed. Of the three TCRVβ CARs exemplified, best in vitro killing was observed against TCRVβ1 and 2- and less so for TCRVβ9-expressing targets. This pattern correlated with the intensity of staining of T cells by the corresponding mAbs, suggesting that the anti-TCRVβ9 mAb and thus the corresponding CAR are of lower affinity, highlighting the need to select high affinity mAb.

The robust anti-TCL activity of anti-TCRVβ CARs was shown using two effector platforms: T and iNKT cells thus offering the prospect of either autologous or allogeneic, off-the-shelf cellular immunotherapy respectively. The subcutaneous TCL model used to test the in vivo efficacy of TCRVβ CAR-INKT cells is faithful to human TCLs in which skin is often the primary or secondary site of disease. Although potential toxicity of allogeneic CAR-iNKT cells will be ultimately determined in clinical trials, early clinical experience with allogeneic CAR-iNKT cells against B cell lymphoma suggested absence of significant toxicity and aGVHD^{30,31}. The data also suggest that CAR-iNKT cells would offer additional therapeutic advantages over CAR-T cells in TCRVB and CD1d co-expressing cases of T cell malignancies such as T lymphoblastic lymphoma, as exemplified by the Jurkat T cell line. Incorporation of αGalCer in the therapeutic armamentarium could result in further enhancement of the CAR-iNKT cell-mediated anti-leukaemia/lymphoma effect.

Reassuringly, anti-TCRVβ CAR-iNKT immunotherapy does not appear to impact adaptive anti-viral immunity: there was no change in the frequency of flu-specific or HTLV-1 Tax-specific CD8+ T cells when PBMC from HTLV-1 carriers were cultured with and without anti-TCRVβ CAR-INKT. The inventors also evaluated the effect of CAR-iNKT activity on proviral expression by by-standing HTLV-1 infected CD4+ T cells and observed no difference in HTLV-1 Tax expression in the presence or absence of CAR-iNKT cells. Thus, at least in short term in vitro assays, the inventors have excluded the possibility that bystander activation of HTLV-1 infected T cells favours HTLV-1 proviral reactivation.

### Summary

In the present disclosure, the inventors have successfully demonstrated that the novel and innovative anti-TCRVβ CARs precision medicine approach for the treatment of TCL and ATL disclosed herein, can be applied to all TCRVβ chains. Because of their distinct advantages discussed above, the use of the allogeneic iNKT cell platform as the effector cell, which will allow the swift deployment of pre-manufactured allogeneic anti-TCRVβCAR-INKT cells in HTLV-1 endemic areas and in combination with advanced clinical trial designs such as Bayesian optimal interval design, is preferred. Such off-the-shelf, anti-TCRVβ CAR-iNKT cells can be tested not only in patients with ATL but also in HTLV-1-infected individuals in whom, as the inventors recently showed, clonal T cell expansions above a certain threshold portend high risk of progression to ATL, a highly incurable cancer^{32,33}.

### REFERENCES

1. Cancer Research UK. Cancer Statistics for the UK. Available at: https://www.cancerresearchuk.org/health-professional/cancer-statistics-for-the-uk. (Accessed: 18 Oct. 2021)
2. Watanabe, T. Current status of HTLV-1 infection. Int. J. Hematol. 94, 430-4 (2011).
3. Cook, L. B. M. & Taylor, G. P. HTLV-1: the silent impact revealed. Lancet Infect. Dis. (2019). doi:10.1016/S1473-3099(19)30432-3
4. Tembhare, P. et al. Flow cytometric immunophenotypic assessment of T-cell clonality by Vβ repertoire analysis: detection of T-cell clonality at diagnosis and monitoring of minimal residual disease following therapy. Am. J. Clin. Pathol. 135, 890-900 (2011).
5. Tembhare, P. et al. Flow cytometric immunophenotypic assessment of T-cell clonality by vβ repertoire analysis in fine-needle aspirates and cerebrospinal fluid. Am. J. Clin. Pathol. 137, 220-6 (2012).
6. Rowan, A. G. et al. T Cell Receptor VB Staining Identifies the Malignant Clone in Adult T cell Leukemia and Reveals Killing of Leukemia Cells by Autologous CD8+ T cells. PLOS Pathog. 12, e1006030 (2016).
7. June, C. H. & Sadelain, M. Chimeric Antigen Receptor Therapy. N. Engl. J. Med. 379, 64-73 (2018).
8. Fleischer, L. C., Spencer, H. T. & Raikar, S. S. Targeting T cell malignancies using CAR-based immunotherapy: challenges and potential solutions. J. Hematol. Oncol. 12, 141 (2019).
9. Maciocia, P. M. et al. Targeting the T cell receptor β-chain constant region for immunotherapy of T cell malignancies. Nat. Med. 23, (2017).
10. Nikolich-Zugich, J., Slifka, M. K. & Messaoudi, I. The many important facets of T-cell repertoire diversity. Nat. Rev. Immunol. 4, 123-32 (2004).
11. Karadimitris, A. et al. Human CD1d-glycolipid tetramers generated by in vitro oxidative refolding chromatography. Proc. Natl. Acad. Sci. U.S.A. 98, 3294-8 (2001).
12. Exley, M. A., Wilson, S. B. & Balk, S. P. Isolation and Functional Use of Human NKT Cells. Curr. Protoc. Immunol. 119, 14.11.1-14.11.20 (2017).
13. Godfrey, D. I. & Kronenberg, M. Going both ways: immune regulation via CD1d-dependent NKT cells. J. Clin. Invest. 114, 1379-88 (2004).
14. Dellabona, P., Padovan, E., Casorati, G., Brockhaus, M. & Lanzavecchia, A. An invariant V alpha 24-J alpha Q/V beta 11 T cell receptor is expressed in all individuals by clonally expanded CD4-8-T cells. J. Exp. Med. 180, 1171-6 (1994).
15. Exley, M. et al. CD1d structure and regulation on human thymocytes, peripheral blood T cells, B cells and monocytes. Immunology 100, 37-47 (2000).
16. Kawano, T. et al. CD1d-restricted and TCR-mediated activation of valpha14 NKT cells by glucosylceramides. Science 278, 1626-9 (1997).
17. Brossay, L. et al. CD1d-mediated recognition of an alpha-galactosylceramide by natural killer T cells is highly conserved through mammalian evolution. J. Exp. Med. 188, 1511-8 (1998).
18. Slauenwhite, D. & Johnston, B. Regulation of NKT Cell Localization in Homeostasis and Infection. Front. Immunol. 6, 255 (2015).
19. Johnston, B., Kim, C. H., Soler, D., Emoto, M. & Butcher, E. C. Differential chemokine responses and homing patterns of murine TCR alpha beta NKT cell subsets. J. Immunol. 171, 2960-9 (2003).
20. Kim, C. H., Johnston, B. & Butcher, E. C. Trafficking machinery of NKT cells: shared and differential chemokine receptor expression among V alpha 24(+) V beta 11(+) NKT cell subsets with distinct cytokine-producing capacity. Blood 100, 11-6 (2002).
21. Salio, M., Silk, J. D., Yvonne Jones, E. & Cerundolo, V. Biology of CD1- and MR1-Restricted T Cells. Annu. Rev. Immunol. 32, 323-366 (2014).
22. Chaudhry, M. S. & Karadimitris, A. Role and regulation of CD1d in normal and pathological B cells. J. Immunol. 193, 4761-8 (2014).
23. Gumperz, J. E., Miyake, S., Yamamura, T. & Brenner, M. B. Functionally distinct subsets of CD1d-restricted natural killer T cells revealed by CD1d tetramer staining. J. Exp. Med. 195, 625-36 (2002).
24. Lee, P. T. et al. Testing the NKT cell hypothesis of human IDDM pathogenesis. J. Clin. Invest. 110, 793-800 (2002).
25. Chaidos, A. et al. Graft invariant natural killer T-cell dose predicts risk of acute graft-versus-host 25. disease in allogeneic hematopoietic stem cell transplantation. Blood 119, 5030-6 (2012).
26. Rubio, M.-T. et al. Pre-transplant donor CD4− invariant NKT cell expansion capacity predicts the occurrence of acute graft-versus-host disease. Leukemia 31, 903-912 (2017).
27. Schneidawind, D. et al. Third-party CD4+ invariant natural killer T cells protect from murine GVHD lethality. Blood 125, 3491-3500 (2015).
28. Mavers, M., Maas-Bauer, K. & Negrin, R. S. Invariant Natural Killer T Cells As Suppressors of Graft-versus-Host Disease in Allogeneic Hematopoietic Stem Cell Transplantation. Front. Immunol. 8, 900 (2017).
29. Rotolo, A. et al. Enhanced Anti-lymphoma Activity of CAR19-INKT Cells Underpinned by Dual CD19 and CD1d Targeting. Cancer Cell 34, 596-610.e11 (2018).
30. Exley, M. A. et al. Adoptive Transfer of Invariant NKT Cells as Immunotherapy for Advanced Melanoma: A Phase I Clinical Trial. Clin. Cancer Res. 23, 3510-3519 (2017).
31. Heczey, A. et al. Anti-GD2 CAR-NKT cells in patients with relapsed or refractory neuroblastoma: an interim analysis. Nat. Med. 26, 1686-1690 (2020).
32. Rowan, A. G. et al. Evolution of retrovirus-infected premalignant T-cell clones prior to adult T-cell leukemia/lymphoma diagnosis. Blood 135, 2023-2032 (2020).
33. Wolf, S. N. et al. Quantification of T cell clonality in human T cell leukaemia virus type-1 carriers can detect the development of adult T cell leukaemia early. Blood Cancer J. 11, 66 (2021).

1. A chimeric antigen receptor (CAR) construct specific for a T-cell receptor (TCR) beta-chain variable region (Vβ) subunit of a T cell.

2. A CAR construct according to claim 1, which is specific for a pathogenic T cell.

3. A CAR construct according to claim 2, wherein the pathogenic T cell is either (i) a malignant, pathogenic T cell, or (ii) a non-malignant, pathogenic T cell.

4. A CAR construct according to any preceding claim, wherein the Vβ subunit is selected from a group of Vβ subunits shown in Table 1.

5. A CAR construct according to any preceding claim, wherein the CAR construct targets a TCR Vβ subunit on a T-cell selected from a group consisting of the following Vβ subunits: TCR-Vβ1, TCR-Vβ2, TCR-Vβ9 and TCR-Vβ11.

6. A CAR construct according to any preceding claim, wherein the CAR construct is specific for TCR-Vβ1, optionally wherein the TCR-Vβ1 subunit comprises an amino acid sequence substantially as set out in SEQ ID No:1, or a variant or fragment thereof.

7. A CAR construct according to any preceding claim, wherein the CAR construct is specific for TCR-Vβ2, optionally wherein the TCR-Vβ2 subunit comprises an amino acid sequence substantially as set out in SEQ ID No:2, or a variant or fragment thereof.

8. A CAR construct according to any preceding claim, wherein the CAR construct is specific for TCR-Vβ9, optionally wherein the TCR-Vβ9 subunit comprises an amino acid sequence substantially as set out in SEQ ID No:3, or a variant or fragment thereof.

9. A CAR construct according to any preceding claim, wherein the CAR construct is specific for TCR-Vβ11, optionally wherein the TCR-Vβ11 subunit comprises an amino acid sequence substantially as set out in SEQ ID No:4, or a variant or fragment thereof.

10. A CAR construct according to any preceding claim, wherein the CAR construct comprises:
a) a signalling peptide;
b) an antigen-binding domain or moiety specific for the TCR-Vβ subunit, preferably an anti-TCRVβ antibody or a functional fragment thereof specific for the TCR-Vβ subunit, and more preferably a single-chain variable fragment (scFv) domain of an anti-TCRVβ antibody;
c) a hinge region, preferably comprising a transmembrane domain, and most preferably a transmembrane domain and cytoplasmic region;
d) a primary stimulatory domain; and/or
e) a co-stimulatory domain, preferably two or more co-stimulatory domains.

11. A CAR construct according to any preceding claim, wherein the CAR construct comprises a signalling peptide, which comprises human CD8α, or a fragment or variant thereof.

12. A CAR construct according claim 11, the CAR construct comprises a signalling peptide comprising an amino acid sequence substantially as set out in SEQ ID No:5, or a fragment or variant thereof and/or wherein the signalling peptide is encoded by a nucleotide sequence substantially as set out in SEQ ID No: 6, or a fragment or variant thereof.

13. A CAR construct according to any preceding claim, wherein the CAR construct comprises an antigen-binding domain or moiety specific for the TCR-Vβ subunit, wherein the antigen-binding domain or moiety comprises an anti-TCRVβ antibody or a functional fragment thereof that is specific for the TCR-Vβ subunit, optionally wherein the antigen-binding domain specific for the TCR-Vβ subunit comprises a single-chain variable fragment (scFv) domain of an anti-TCRVβ antibody.

14. A CAR construct according to claim 13, wherein the CAR construct comprises a scFv comprising a VL and/or a VH from an anti-TCRVβ1 antibody, wherein the CAR construct: —
(i) comprises a VL chain having an amino acid sequence substantially as set out in SEQ ID No:7, or a fragment or variant thereof and/or the VL chain is encoded by a nucleotide sequence substantially as set out in SEQ ID No: 8, or a fragment or variant thereof; and/or
(ii) comprises a VH chain having an amino acid sequence substantially as set out in SEQ ID No:10, or a fragment or variant thereof and/or the VH chain is encoded by a nucleotide sequence substantially as set out in SEQ ID No: 11, or a fragment or variant thereof.

15. A CAR construct according to any preceding claim, wherein the CAR construct comprises a scFv comprising a VL and/or a VH from an anti-TCRVβ2 antibody, wherein the CAR construct: —
(i) comprises a VL chain having an amino acid sequence substantially as set out in SEQ ID No:11, or a fragment or variant thereof and/or the VL chain is encoded by a nucleotide sequence substantially as set out in SEQ ID No: 12, or a fragment or variant thereof; and/or
(ii) comprises a VH chain having an amino acid sequence substantially as set out in SEQ ID No:13, or a fragment or variant thereof and/or the VH chain is encoded by a nucleotide sequence substantially as set out in SEQ ID No: 14, or a fragment or variant thereof.

16. A CAR construct according to any preceding claim, wherein the CAR construct comprises a scFv comprising a VL and/or a VH from an anti-TCRVβ9 antibody, wherein the CAR construct: —
(i) comprises a VL chain having an amino acid sequence substantially as set out in SEQ ID No:15, or a fragment or variant thereof and/or the VL chain is encoded by a nucleotide sequence substantially as set out in SEQ ID No: 16, or a fragment or variant thereof; and/or
(ii) comprises a VH chain having an amino acid sequence substantially as set out in SEQ ID No:17, or a fragment or variant thereof and/or the VH chain is encoded by a nucleotide sequence substantially as set out in SEQ ID No: 18, or a fragment or variant thereof.

17. A CAR construct according to any preceding claim, wherein the CAR construct comprises a scFv comprising a VL and/or a VH from an anti-TCRVβ11 antibody, wherein the CAR construct: —
(i) comprises a VL chain having an amino acid sequence substantially as set out in SEQ ID No:19, or a fragment or variant thereof and/or the VL chain is encoded by a nucleotide sequence substantially as set out in SEQ ID No: 20, or a fragment or variant thereof; and/or
(ii) comprises a VH chain having an amino acid sequence substantially as set out in SEQ ID No:21, or a fragment or variant thereof and/or the VH chain is encoded by a nucleotide sequence substantially as set out in SEQ ID No: 22, or a fragment or variant thereof.

18. A CAR construct according to any one of claims 14-17, wherein the VH and VL sequences are separated by at least one linker sequence comprising an amino acid sequence substantially as set out in SEQ ID No:23, or a fragment or variant thereof.

19. A CAR construct according to any preceding claim, wherein the CAR construct comprises a hinge domain, which comprises: (i) an extracellular domain, or a portion thereof; (ii) a transmembrane (TM) domain, or a portion thereof; and (iii) a cytoplasmic domain, or a portion thereof.

20. A CAR construct according to claim 19, wherein: —
(i) the hinge domain comprises a CD8α sequence, or a portion thereof, and more preferably a human CD8α sequence, or a portion thereof;
(ii) the hinge domain comprises an amino acid sequence substantially as set out in SEQ ID No: 24, or a fragment or variant thereof;
(iii) the hinge comprises or consists of an amino acid sequence substantially as set out in SEQ ID No:25, or a fragment or variant thereof; and/or
(iv) the hinge domain is encoded by a nucleotide sequence substantially as set out in SEQ ID No: 26, or a fragment or variant thereof.

21. A CAR construct according to claim 20, wherein the hinge domain comprises or is derived from a human CD8α sequence, or a portion thereof comprising an amino acid sequence substantially as set out in SEQ ID No: 51, or a fragment or variant thereof.

22. A CAR construct according to claim 21, wherein:
(i) the hinge domain comprises an extracellular domain of human CD8α or a portion thereof defined by amino acids 138-182 of human CD8α, or a portion thereof, as represented by SEQ ID No: 51;
(ii) the hinge domain is at least one, two or three amino acids longer than amino acids 138-182 of human CD8α on the N-terminus, as represented by SEQ ID No: 51;
(iii) the hinge domain is at least four, five or six amino acids longer amino acids 138-182 of human CD8α on the N-terminus, as represented by SEQ ID No: 51; and/or
(iv) the hinge domain is at least seven, eight, nine or ten amino acids longer amino acids 138-182 of human CD8α on the N-terminus, as represented by SEQ ID No: 51.

23. A CAR construct according to claim 22, wherein:
(i) the hinge domain comprises the extracellular domain of human CD8α or a portion thereof defined by amino acids 137-182, 136-182, or 135-182 of human CD8α, as represented by SEQ ID No: 51;
(ii) the hinge domain comprises the extracellular domain of human CD8α or a portion thereof defined by amino acids 134-182, 133-182, or 132-182 of human CD8α, as represented by SEQ ID No: 51; and/or
(iii) the hinge domain comprises the extracellular domain of human CD8α or a portion thereof defined by amino acids 131-182, 130-182, 129-182 or 128-182 of human CD8α, as represented by SEQ ID No: 51.

24. A CAR construct according to claim 21, wherein the hinge domain comprises a cytoplasmic domain of human CD8α or a portion thereof defined by amino acids 204-206 of human CD8α, or a portion thereof, as represented by SEQ ID No: 51.

25. A CAR construct according to claim 24, wherein:
(i) the cytoplasmic domain is at least one amino acid longer than amino acids 204-206 of human CD8α on the C-terminus, as represented by SEQ ID No: 51;
(ii) the cytoplasmic domain is at least two amino acids longer than amino acids 204-206 of human CD8α on the C-terminus, as represented by SEQ ID No: 51;
(iii) the cytoplasmic domain is at least three amino acids longer than amino acids 204-206 of human CD8α on the C-terminus, as represented by SEQ ID No: 51; and/or
(iv) the cytoplasmic domain is at least four amino acids longer than amino acids 204-206 of human CD8α on the C-terminus, as represented by SEQ ID No: 51.

26. A CAR construct according to claim 25, wherein:
(i) the cytoplasmic domain is defined by amino 204-207 of human CD8α of human CD8α, as represented by SEQ ID No: 51;
(ii) the cytoplasmic domain is defined by amino 204-208 of human CD8α of human CD8α, as represented by SEQ ID No: 51;
(iii) the cytoplasmic domain is defined by amino 204-209 of human CD8α of human CD8α, as represented by SEQ ID No: 51; and/or
(iv) the cytoplasmic domain is defined by amino 204-210 of human CD8α of human CD8α, as represented by SEQ ID No: 51.

27. A CAR construct according to claim 19, wherein the CAR construct comprises a hinge domain, which comprises:
(i) a human CD8α extracellular domain, or a portion thereof having an amino acid sequence substantially as set out in SEQ ID No: 24, or a fragment or variant thereof;
(ii) a human CD8α transmembrane (TM) domain, or a portion thereof having an amino acid sequence substantially as set out in SEQ ID No: 49, or a fragment or variant thereof; and
(iii) a human CD8α cytoplasmic domain, or a portion thereof having an amino acid sequence substantially as set out in SEQ ID No: 50, or a fragment or variant thereof.

28. A CAR construct according to any preceding claim, wherein the CAR construct comprises an intracellular domain, which comprises a primary stimulatory CD3ζ chain and a co-stimulatory domain of CD28.

29. A CAR construct according to claim 28, wherein the CAR construct comprises a co-stimulatory domain of CD28 having an amino acid sequence substantially as set out in SEQ ID No:27, or a fragment or variant thereof and/or wherein the co-stimulatory domain of CD28 is encoded by a nucleotide sequence substantially as set out in SEQ ID No: 28, or a fragment or variant thereof.

30. A CAR construct according to either claim 28 or claim 29, wherein the CAR construct comprises one or two co-stimulatory domains, optionally selected from a CD28, 4-1BB signalling domain and OX40 signalling domain.

31. A CAR construct according to any one of claims 28-30, wherein the CAR construct comprises a stimulatory protein CD3ζ having an amino acid sequence substantially as set out in SEQ ID No: 29, or a fragment or variant thereof and/or wherein the stimulatory protein CD3ζ is encoded by a nucleotide sequence substantially as set out in SEQ ID No: 30, or a fragment or variant thereof.

32. A CAR construct according to any preceding claim, wherein the CAR construct comprises an amino acid sequence substantially as set out in SEQ ID No:31 or 33, or a fragment or variant thereof; and/or is encoded by a nucleotide sequence substantially as set out in SEQ ID No: 32 or 34, or a fragment or variant thereof.

33. A CAR construct according to any preceding claim, wherein the CAR construct comprises an amino acid sequence substantially as set out in SEQ ID No:35 or 37, or a fragment or variant thereof; and/or is encoded by a nucleotide sequence substantially as set out in SEQ ID No: 36 or 38, or a fragment or variant thereof.

34. A CAR construct according to any preceding claim, wherein the CAR construct comprises an amino acid sequence substantially as set out in SEQ ID No:39 or 41, or a fragment or variant thereof; and/or is encoded by a nucleotide sequence substantially as set out in SEQ ID No: 40 or 42, or a fragment or variant thereof.

35. A CAR construct according to any preceding claim, wherein the CAR construct comprises an amino acid sequence substantially as set out in SEQ ID No:43 or 45, or a fragment or variant thereof; and/or is encoded by a nucleotide sequence substantially as set out in SEQ ID No: 44 or 46, or a fragment or variant thereof.

36. A nucleic acid encoding the CAR construct according to any one of claims 1-35.

37. An expression vector or a plasmid encoding the CAR construct according to any one of claims 1-35, or comprising the nucleic acid according to claim 36.

38. An effector cell expressing the CAR construct according to any one of claims 1-35, or comprising the nucleic acid according to claim 36 or the vector according to claim 37.

39. The effector cell according to claim 38, wherein the effector cell is (i) a normal or conventional αβT-cell, or (ii) an innate lymphocyte, optionally an invariant natural killer T (INKT) cell, γδT cell or NK cell.

40. The effector cell according to either claim 38 or claim 39, wherein the effector cell is an iNKT cell.

41. A method of producing an effector cell expressing an anti-TCRVβ CAR, the method comprising transducing an effector cell with the nucleic acid according to claim 29 or the vector according to claim 37, such that the effector cell expresses an anti-TCRVβ CAR.

42. The method according to claim 41, wherein the effector cell is (i) a normal or conventional αβT-cell, or (ii) an innate lymphocyte, preferably an invariant natural killer T (iNKT) cell, γδT cell or NK cell.

43. The method according to either claim 41 or claim 42, wherein the method comprises an initial step of isolating the effector cell from peripheral blood cells (PBCs), and the effector cell is activated with one or both of CD3 and CD28 antibodies.

44. The method according to any one of claims 41-43, wherein the effector cell is activated with an interleukin, preferably wherein the interleukin is IL-15.

45. A pharmaceutical composition comprising a therapeutically effective amount of the CAR construct according to any one of claims 1-35, the nucleic acid according to claim 29, the vector according to claim 37, or the effector cell according to any one of claims 38-40, and a pharmaceutically acceptable excipient.

46. The pharmaceutical composition according to claim 45, wherein the pharmaceutical composition comprises a plurality of the effector cell, preferably a T cell, or iNKT cell, and wherein the composition comprises at least 100, 1000, 10,000, 100,000, 1,000,000 or at least 10,000,000 effector cells.

47. The CAR construct according to any one of claims 1-35, the nucleic acid according to claim 36, the vector according to claim 37, or the effector cell according to any one of claims 38-40, for use in therapy or diagnosis.

48. The CAR construct according to any one of claims 1-35, the nucleic acid according to claim 36, the vector according to claim 37, or the effector cell according to any one of claims 38-40, for use in (i) immunotherapy; (ii) for treating, preventing or ameliorating cancer; (iii) for treating, preventing or ameliorating an autoimmune disease; or (iv) for treating, preventing or ameliorating any disease characterised by the presence of pathogenic T cells.

49. The CAR construct according to any one of claims 1-35, the nucleic acid according to claim 36, the vector according to claim 37, or the effector cell according to any one of claims 38-40, for use according to either claim 47 or claim 48, wherein the cancer is a T-cell malignancy, which is a solid tumour or a liquid tumour.

50. The CAR construct according to any one of claims 1-35, the nucleic acid according to claim 36, the vector according to claim 37, or the effector cell according to any one of claims 38-40, for use according to any one of claims 47-49, wherein the T-cell malignancy is selected from a group consisting of: a hematolymphoid neoplasm; a precursor T-cell neoplasm; T-lymphoblastic leukaemia/lymphoma; mature T-cell neoplasms; Peripheral T cell lymphoma (PRCL); mature T-cell leukaemias; Primary cutaneous T-cell lymphoid proliferations and lymphomas; Intestinal T-cell lymphoid proliferations and lymphomas; Hepatosplenic T-cell lymphoma; Anaplastic large cell lymphoma; Nodal T-follicular helper (TFH) cell lymphoma; Peripheral T-cell lymphoma, NOS; EBV-positive NK-cell and T-cell lymphomas; EBV-positive T-cell lymphoid proliferations; Subcutaneous Panniculitis-Like Lymphoma (SPTCL); Angioimmunoblastic T-cell lymphoma (AITL); and Cutaneous T-cell lymphoma (CTCL).

51. The CAR construct according to any one of claims 1-35, the nucleic acid according to claim 36, the vector according to claim 37, or the effector cell according to any one of claims 38-40, for use according to either claim 47 or 48, wherein the autoimmune disease is one caused by pathogenic autoreactive T cells, or selected from a group consisting of: systemic lupus erythematosus, rheumatoid arthritis, and myasthenia gravis.

51. A process for making the pharmaceutical composition according to either claim 45 or claim 46, the process comprising combining a therapeutically effective amount of the CAR construct according to any one of claims 1-35, the nucleic acid according to claim 36, the vector according to claim 37, or the effector cell according to any one of claims 38-40, and a pharmaceutically acceptable vehicle.
